SEQUENCE LISTING (39 sequences)

SEQ ID NO: 1 - protein, 63 aa, Homo sapiens
MFAMKTKAALAIWCPGYSETQINATQAMKKRRKRKVTTNKCLEQVSQLQGLWRRFNRPLLKQQ

SEQ ID NO: 2 - protein, 159 aa, Homo sapiens
MFPFALLYVLSVSFRKIFILQLVGLVLTYDFTNCDFEKIKAAYLSTISKDLITYMSGTKSTEFNNTVSCSNRPHCLTEIQSLTFNPTAGCASLAKEMFAMKTKAALAIWCPGYSETQINATQAMKKRRKRKVTTNKCLEQVSQLQGLWRRFNRPLLKQQ

SEQ ID NO: 3 - DNA, 192 bp, Homo sapiens
atgttcgccatgaaaactaaggctgccttagctatctggtgcccaggctattcggaaactcagataaatgctactcaggcaatgaagaagaggagaaaaaggaaagtcacaaccaataaatgtctggaacaagtgtcacaattacaaggattgtggcgtcgcttcaatcgacctttactgaaacaacagtaa

SEQ ID NO: 4 - DNA, 480 bp, Homo sapiens
atgttcccttttgccttactatatgttctgtcagtttctttcaggaaaatcttcatcttacaacttgtagggctggtgttaacttacgacttcactaactgtgactttgagaagattaaagcagcctatctcagtactatttctaaagacctgattacatatatgagtgggaccaaaagtaccgagttcaacaacaccgtctcttgtagcaatcggccacattgccttactgaaatccagagcctaaccttcaatcccaccgccggctgcgcgtcgctcgccaaagaaatgttcgccatgaaaactaaggctgccttagctatctggtgcccaggctattcggaaactcagataaatgctactcaggcaatgaagaagaggagaaaaaggaaagtcacaaccaataaatgtctggaacaagtgtcacaattacaaggattgtggcgtcgcttcaatcgacctttactgaaacaacagtaa

SEQ ID NO: 5 - DNA, 21 bp, Artificial Sequence
  synthetic primer
caccgtctcttgtagcaatcg

SEQ ID NO: 6 - DNA, 20 bp, Artificial Sequence
  synthetic primer
tagcctgggcaccagatagc

SEQ ID NO: 7 - DNA, 18 bp, Artificial Sequence
  synthetic primer
ccgcctatgagcagccac

SEQ ID NO: 8 - DNA, 21 bp, Artificial Sequence
  synthetic primer
cctgagtagcatttatctgag

SEQ ID NO: 9 - protein, 176 aa, Cricetulus griseus
MGEYGPYVDWGVLLRDLLILQVVQLVLAYNFSNCNFEEISNMYSEAIYPDLFEYLNGSLFEQNEVCDNSVSEVVGCAGFPRGGRQPGRELGKTTQKTDCLMKIEYLTLNPIPGCPSLPREIFALHTKATLISQCPGYSETQRNNAQEMKLEVKDICLNQTSQIQFLWHSLLQTLKY

SEQ ID NO: 10 - protein, 143 aa, Equus caballus
MVFFRKIFILQLVGLVLTYDFTDCNFAKIEEEYQNVIFLALKEYMNGIKSTQFNHTVYCGDRPSCLTEIERLTFKSSQCLSLAKKIVTATNATLNSHCPGHSGIQINNTQAMKKRKKREVTTNKCLKQVSNLIELWRYFSRSQ

SEQ ID NO: 11 - protein, 157 aa, Saimiri boliviensis
MKNLGQSKKEEVSFRKIFILQLVGLVLTYDFTNCDFMEIKKEYWRTIGEDLRTYMSGTKSTEFNTTIFCNNRSHCLTEIQRLTFNPTLGCASLAKELFAVRTNATLALWCPGYSETQINDTQAMKKRKKRKVTTNKCLEQVSQLQGLWRRFIRTLRK

SEQ ID NO: 12 - protein, 157 aa, Callithrix jacchus
MKNLGQSKKEKVSFRKIFILQLVGLVLTYDFTNCDFTKIKKEYCRTILEDLLTYMIGTKSTEFNNTIFCNNRSHCLTEIQRLTFDPTPGCAALAKELFAVRTNATLALWCPGYSETQINATQEMKKRKKRKVTTNKCLEQVSQLSGLWRRFIRTLRK

SEQ ID NO: 13 - protein, 159 aa, Macaca mulatta
MKSLGQSKKEEVSFRKFFIFQLVGLVLTYDFTNCDFEKIEADYLRTISKDLITYMSGTKSTDFNNTVSCSNRPHCLTEIQSLTFNPTPRCASLAKEMFARKTKATLALWCPGYSETQINATQAMKKRRKRKVTTNKCLEQVSQLLGLWRRFIRTLLKKQ

SEQ ID NO: 14 - protein, 159 aa, Unknown
  PREDICTED thymic stromal lymphopoietin [Papio anubis].
MKSLGQSKKEEVSFRKFFIFQLVGLVLTYDFTNCNFEKIEADYLSTISKDLITYMSGTKSTDFNNTVSCSNRPHCLTEIQSLTFNPTPRCASLAKEMFARKTKATLALWCPGYSETQINATQAMKKRRKRKVTTNKCLEQVSQLLGLWRRFIRTLLKKQ

SEQ ID NO: 15 - protein, 159 aa, Unknown
  PREDICTED thymic stromal lymphopoietin [Nomascus leucogenys]
MKCLGQSKKEEVSFRKIFILQLVGLVLTYDFTNCDFEKIKAAYLSTISKDLITYMSGTKSTEFNNTVSCSNRPHCLTEIQSLTFNRTAGCVSLAKEMFAMKTKAALALWCPGYSETQINATQAMKKRRKRKVTTNKCLEQVSQLLGLWRRFSRRLLKQQ

SEQ ID NO: 16 - protein, 159 aa, Unknown
  PREDICTED thymic stromal lymphopoietin [Pongo abelii]
MKCLGQSKKEEVSFRKIFILQLVGLVLTYDFTNCDFEKIKAAYLSTISKDLITYMSGTKSTEFNNTVSCSNRPHCLTEIQSLTFNPTAGCASLAKEVFAMKTKAALALWCPGYSETQINATQAMKKRRKRKVTTNKCLEQVSQLLGLWRRFNRPLLKQQ

SEQ ID NO: 17 - protein, 183 aa, Felis catus
MALQEKERQFSVGGKTLVRNRGGTEFPSILPDALLFVLSVFFRKIFILQLVGLVLTYNFTDCDFGKIRKKYQEVIYQALNKYMNGVKSTEFNNYIYCEDPPDCLAKIDLITFYPTHGCTTLAKEIFAIRTNATLTLQCPGYSGTQINNTQAKKKRKKRQVTTNKCREQVSHLMELWRRFSRIS

SEQ ID NO: 18 - protein, 159 aa, Pan troglodytes
MFPFALLYVLSVSFRKIFILQLVGLVLTYDFTNCDFEKIKAAYLSTISKDLITYMSGTKSTEFNNTVSCSNRPHCLTEIQSLTFNPTAGCASLAKEMFAMKTKAALTIWCPGYSETQINATQAMKKRRKRKVTTNKCLEQVSQLQGLWRRFNRPLLKQQ

SEQ ID NO: 19 - protein, 159 aa, Pan paniscus
MKCLGQSKKEEVSFRKIFILQLVGLVLTYDFTNCDFEKIKAAYLSTISKDLITYMSGTKSTEFNNTVSCSNRPHCLTEIQSLTFNPTAGCASLAKEMFAMKTKAALAIWCPGYSETQINATQAMKKRRKRKVTTNKCLEQVSQLQGLWRRFNRPLLKQQ

SEQ ID NO: 20 - protein, 105 aa, Gorilla gorilla
MSGTKSTEFNNTVSCSNRPHCLTEIQSLTFNPTAGCASLAKEMFAMKTKAALAIWCPGYSETQINATQAMKKRRKRKVTTNKCLEQVSQLQGLWRRFNRPLLKQQ

SEQ ID NO: 21 - protein, 154 aa, Canis lupus
LPDALLSVLSVFFRKIFVLQLVGLVLTYNFIDCDFEKIRWKYQEVIYQALEKYMDGTRSTEFSHPVYCADPPDCLARIERLTLHRIRGCASGAREAFAEGTVAALAAECPGYAAAPINNTQAKKKRKKRGVTTNKCREQVAHLIGLWRRFSRIS

SEQ ID NO: 22 - protein, 148 aa, Ailuropoda melanoleuca
FVLSVFFRKIFILQLVGLVLTYNFTGCDFEKIRKKYQEVIYQALEKYMNGTKSTEFNNPIYCEDPPDCLAKIERITFHPTHDCTSLAEEIFAIGTRATLTLQCPSYSGMQINNTQAKKKRKKRKVTTNKCREQVAYLIGLWRRFSRIS

SEQ ID NO: 23 - protein, 140 aa, Mus musculus
MVLLRSLFILQVLVRMGLTYNFSNCNFTSITKIYCNIIFHDLTGDLKGAKFEQIEDCESKPACLLKIEYYTLNPIPGCPSLPDKTFARRTREALNDHCPGYPETERNDGTQEMAQEVQNICLNQTSQILRLWYSFMQSPE

SEQ ID NO: 24 - protein, 214 aa, Bos taurus
MTKEGRKIFILQLVGLVLTYNFTDCDFKKIKESYRNIIYQELNNYTKGTKAIDFDHFVYCEDQPDCLSKIQSLTFEGRPGCAAPAREAFAVRTHAALAAACPGYRGPQVSRATQSARRVPRASERGAGPGEPRPPAARRRAWTRALTSEGRSGGNLRAAESPLAPTSAGALTLWAGINNTQTMRKIRKRQIKRKECLEQVTYLKELWQRLSRIS

SEQ ID NO: 25 - DNA, 531 bp, Cricetulus griseus
atgggtgaatatggaccttatgtggactggggagttcttctcagggacctcctcatcttgcaagtggtacagttggtgctagcttacaatttttctaattgtaacttcgaggagatttcaaatatgtattctgaagcaatttatcctgacctgtttgaatatttgaatgggagtctgttcgaacaaaacgaggtctgtgacaactcggtgagtgaagttgttggctgtgctggctttccccggggtggcaggcaaccgggcagggagctggggaagaccacccagaagacagattgtctcatgaaaatcgagtaccttactctcaatcctatcccgggctgcccgtcactcccccgggaaatattcgccttgcatacgaaagcgaccctcattagtcaatgcccaggctactcagaaactcagagaaacaatgctcaggaaatgaagctagaagtcaaagacatctgcctgaatcaaacctcacaaatacagttcttgtggcattcccttcttcaaactctaaaatactaa

SEQ ID NO: 26 - DNA, 649 bp, Equus caballus
actcccaacttaaggcaacagcatggtttttttcaggaagatcttcatcttgcagctggtagggctggtgctgacctacgatttcactgactgtaactttgcgaagattgaagaggaatatcagaatgtcatttttctagccctgaaggaatatatgaatggaatcaaaagtacccagttcaaccacactgtctactgtggagaccggccgagttgtctcaccgaaatcgagcgccttaccttcaagtccagccaatgcctgtcactcgccaagaaaattgtaacagcaactaacgctacgctcaacagccattgcccaggccactccggaattcagataaataatacccaggcaatgaagaaaagaaagaaaagagaagtcacgacaaataaatgccttaaacaagtctcaaacttaatagaattgtggcgttatttcagtcgctcccagtgaaacaaaataaatctccactgaagtcctatttcacagtaacaaattatctttactaggtagatagaacattaactctgggatgtttaaaagactaccaatggttattttttaattacagaagggtttcttaacttatttttgtaagctttcctcatttaatgtaggtctgtaatgcagggaaagtactaatcctca

SEQ ID NO: 27 - DNA, 474 bp, Saimiri boliviensis
atgaagaatttagggcaaagcaaaaaggaggaagtttctttcaggaaaatcttcatcttacaacttgtagggctggtgctaacttacgacttcactaactgtgactttatggagattaaaaaagagtattggcgtactattggtgaagacttgcgtacatatatgagtgggaccaaaagtaccgagttcaacaccaccatcttttgtaacaatcggtcacactgccttactgaaatccagcgcctgaccttcaatcccaccctcggctgcgcgtcgctggccaaggaactgttcgctgtgagaactaacgctaccctcgctctctggtgcccaggctattcggaaactcagataaatgatactcaggcaatgaagaagaggaaaaaaagaaaagtcacaaccaataagtgtctggaacaagtctcacagttacaaggattgtggcgtcgcttcattcgaactttaaggaaatga

SEQ ID NO: 28 - DNA, 474 bp, Callithrix jacchus
atgaagaatttagggcaaagcaaaaaggagaaagtttctttcaggaaaatcttcatcttacaacttgtagggctggtgctaacttacgacttcactaactgtgactttacgaagattaaaaaagagtattgccgtactattcttgaagacttgcttacatatatgattgggaccaaaagtaccgaattcaacaacaccatcttttgtaacaatcggtcacactgccttactgaaatccagcgcctaaccttcgatcccacccccggctgcgcggcgctcgccaaggaactgttcgccgtgagaactaacgctaccctagctctctggtgcccaggctattcggaaactcagataaatgctactcaggaaatgaagaagaggaaaaaaagaaaagtcacaaccaataagtgtctggaacaagtctcacaattatcaggattgtggcgtcgcttcattcgaactttaaggaaatga

SEQ ID NO: 29 - DNA, 480 bp, Macaca mulatta
atgaagagtttagggcaaagtaaaaaggaggaagtttctttcaggaaattcttcatctttcaacttgtagggctggtgctaacttacgacttcactaactgtgactttgagaagattgaagcagactatctccgtactatttctaaagacctgattacatatatgagtgggaccaaaagtaccgacttcaacaacaccgtctcctgtagcaatcggccacactgccttactgaaatccagagcctaaccttcaatcccaccccccgctgcgcgtcgctcgccaaggaaatgttcgccaggaaaactaaggctaccctcgctctctggtgcccaggctattcggaaactcagataaatgctactcaggcaatgaagaagaggagaaaaaggaaagtcacaaccaataaatgtctggaacaagtgtcacaattactaggattgtggcgtcgcttcattcgaactttactgaaaaaacagtaa

SEQ ID NO: 30 - DNA, 2666 bp, Unknown
  PREDICTED Papio anubis thymic stromal lymphopoietin (TSLP), mRNA
agttggtgatgggggaagaatggacttctttttcaataaatgctgtggtgacttgagtacaacacatgaagaattccagaaaagggccacttttgaagtcagcggtgaatcagaggttttctgaccctcttcagtgccttctatgaaaaagagttcaactctggcttcagtttgtcccttacgcttttccggaatccctcgacatttcctgctctttttcccgggcaccgtatggcctgtgacatttgctgggactcacacacggcgtccaacctccttgctccaagcgatgactcctaggaacttccctagtaccagagcgatgcaggctctgacacgcgagggcggggggcggggtaggggtgggggtgggggatgaacggggaagcggaagggaaggagagagaaaggggacctgggtgtagagagggcccagggcagcatgagccaagctagggcggagcagggggttcgtctcagcaacgtgatgagtgtcagcctttccctcccttcaacgctccattggtgctgctggaactcggaagactgctagacagatatcctccagggcgctctccctgactgcgcttttaagcactcttttctgtatcaacaatagtttgctagcacaaatctgagcccgccatctcgttgcttctcctgtctctaaaacctgtgcccgcgctcaggccccaggtgacagacgttttccagtctacgcggcgcggggctaagcctctgggaggcagagctcaggaaagcgggcgccgcctctgggaaagctgagctcaggacagcatcctctccttgcgctcaccgctaggcacccagcagagcggtctcctcaggtttaacactggctttaagagtgggactgtgaaggtggctgggcactggaatttgtggttttaaaagttcttgacacctactagaaagtgctagaggacccttcagcccctaaggtgcccctagtcaccaagagtaggcgcttttcctgaaattgatatgagaatttgaaaagtaggatttttgagaacaaggcacatcagatctttccccatttatttcaacctcttatcgttcctgcccctaaggttctagtcacggttgcacatttaatgctttcacatggaagtgctgttgaagactgagactgcacccaccctccaaagcatcccataggtaagcacagtttcttactagccaaggagaagggaaagcattagttgaaggcggccacacgaaattctcaaagatgttcaggatgcatgggacagtatgcaaggactccaaatccttacagtggctgctggtttcagaaggtgtttctcccaaataatagaaaggattggtcactgaaacgccctgtgggagaaagacactggtatccatttcttaaaggacattaacacatatgaagagtttagggcaaagtaaaaaggaggaagtttctttcaggaaattcttcatctttcaacttgtagggctggtgctaacttacgacttcactaactgtaactttgagaagattgaagcagactatctcagtactatttctaaagacctgattacatatatgagtgggaccaaaagtaccgacttcaacaacaccgtctcctgtagcaatcggccacactgccttactgaaatccagagcctaaccttcaatcccaccccccgctgcgcgtcgctcgccaaggaaatgttcgccaggaaaactaaggctaccctcgctctctggtgcccaggctattcggaaactcagataaatgctactcaggcaatgaagaagaggagaaaaaggaaagtcacaaccaataaatgtctggaacaagtgtcacaattactaggattgtggcgtcgcttcattcgaactttactgaaaaaacagtaaatcatctttattatggtcatatttcacagcaccaaataaatcatctttattaagtagatgaaacattaactgtgacacagaagatcacaaatagttatcttttaattacagaagagtttcttaacttatttttctaagtttttattgtttaatataaatttataatgcaggggaagtactactcttcaaatgttgagggaagcttccgtaacattgatgactggcttaattggcagtaattctcagccgtaattgcataagcattactcaagaggaaaatccaaaagtgtagcaggagaactcttttccttgaaaaaggaaaaatattgaactaaatgatagtacctaaacttacgtttaaaaggcagacattccttctacatgtaatgacacttcttgtgttaaactaaaaatttacaagagaagaaagtgaaagcaaatggggtttcacaaataactgtaaatatagtgaagcaatttgaaataattttcaagcaaaacattgtgaaagtattctaagctaagttttaaatattacctaacagacaagagtggtatataataagtagatcctgagaagtacctttgttacagctactataaatatatatagagagaatgtactttaatttattttgtgaatgcttttgaaaatgtatatgttcctttgtaattgacactatatatttcttaataaaataattctcaaatttgtttcttacgaatcatctttcaaatctagttagtagacaatttgcacacatacttttct

SEQ ID NO: 31 - DNA, 3840 bp, Unknown
  PREDICTED Nomascus leucogenys thymic stromal lymphopoietin (TSLP), mRNA.
agtggacatttttttcgatgaatgctgtggtgacttgagtacaacacatgaagaattctagaaaagggccatttttgaagtcagcggtgaatcagaggttttctggccctctcgagtgccttctatgaagaagagttcaactctggctccagtttgtcccctactcttttcccgaatccctcgacatttccctgctcttttccccaggtaccgcatggcctgtaacatttgctgggactcacacacggcgtccaatctcctttctccgagcgatgactcctaggaacttcccaaggaccagagcgacgcaggctccgacacgcgacggcggggggcgggggtgggggggtgcgatggacgggggagcgaaagggaaagagagagagaagggacctgggtgtagagagggcccagggccgcatgagccaagccagggcggagcagggggttcatctcagcaacctaatcggtgtcggcctttccctcccttcaaccctccattggtgctgctggaactcggaagactgctagacagacatcttctagggcgctctccctgactcacttggctgcgcttttaagcactcttttctggatcaacaagagtttgctagtaaaaatctgagcccgccatctcgatgcttctcctgtctctaaaactggtgcccgggctgaggcccaggtggcagacgttttccagtctacgctgcgcggggctaagcctctgggaggcagagcgcactaaagcgtgcgccgcctcggggagagctgagctcaggacagcatcctttccgtgcgcgctcgccgctgcgcacccagcagcgctgtctggctccgcttcttgggtcgcgtcctcacgtttaacgctggccttaagagtgggactgtgaaggtggctgggcgctggaatttaagttttaaaagttcttgacacctactagaaagcgctagaggacccttcagcccctaaggcgccggtagtcaccaagagtaggcgcttttcctgaaattgatatgggcaatttgaaaagtagggtttttgagaacaagccacatcagatctttcaccatttatttcaacctcttatcgttcctgcccctaaggttctggtcacggttgaacatttaatgcttccacatggaagtgctgtcgaagactgagactgcacccaccatccaaaacatcccataagtaagcacagtttcttactagccaaggggaagggaaagcactagttgaaagtggccacacgaaatgcgcaaagatgttcagaatgcacgggtcatatgcaaggactctaaatccttacagtggctgctggtttcagaaggtgtttctcccaaatagaaaggattggtcacggaaatgccctgtaggagaaagacactggtatccatttcttaaaggacattcacacttatgaagtgtttagggcaaagcaaaaaggaggaagtttctttcaggaaaatcttcatcttacaacttgtagggctggtgctaacttacgacttcactaactgtgactttgagaagattaaagcagcctatctcagtactatttctaaagacctgattacatatatgagtgggaccaaaagtaccgagttcaacaacaccgtctcctgtagcaatcggccacactgccttactgaaatccagagcctaaccttcaatcgcaccgccggctgcgtgtcactcgccaaagaaatgttcgccatgaaaactaaggctgccttagctctctggtgcccaggctattcggaaactcagataaatgctactcaggcaatgaagaagaggagaaaaaggaaagtcacaaccaataaatgtctggaacaagtgtcacaattactaggattgtggcgtcgcttcagtcgacgtttactgaaacaacagtaaaccatctttattatggtcatatttcacagcaccaaaataaatcatctttattaagtagatgaaacattaactctaactgtgacaaagaagaccacaaatagttatcttttaattacagaagagtttcttaacttatttttttaagtttttattgtgtaagtttataatgcaggggaagtactactcctcaaatgttgagggaagcttccataacattgatgactggcttcatggcagtaattctcggctgtaattgcataagcattgctcaagaggaaaatccaaaagtgcagcaggagaactcttttccctgaaaaaggaaaaatattgaactcaatgatagcaccaaaacttacatttaaaagacagacattccttctacatgtaatgacacttcttgtgttaaaataaaaatttacaagagaagaaagtgaaagcaaatggggtttcataaatagttgtaaatatagtgaaacaatttgaaataattttcaagcaaaatattgtgaaagtattctaagccaagttttaaatattatctaacagatgacagtggtatatacaagtagatcctgagaagtacttttgttacagctactataaatatacatataaattatagaatgtactttattttgtgaacacttttcaaaaggtacattcctttgtaattgacactatatatttcttaataaaacagttctcaaatttgtttcttatgaatcatctttcaaatctagttagacaatttgcacacatacttttctaaggaacattatctttcttcaggtttttacctccactcatccttagagcccactgactgctccgctttatacctgttggccctgcctataggagaaaatatttggagataggcagcttcaggatgcattgcaatcattcttttcttaaattatgtcacgagtcttttctttattcccctcttgaactttcctcacacctggaagaaacaaagtaggcaaaagtgaactggggatgtcaaatcgattcttgaattcctgctgcaagctagagccacaggcaccctctcactcaacttccactcagaaccctctaaacgccagtgggaagggcaacccactgcatgtgggaatgcactgatttttcctaggagtagacatgttcctctaattactccctgagggttagttggggctaaaccatgacagaagtggggaagttcaatgtccttaaatccatcttacctgccaacaggtaagaggaagcttacattatatgtccagtctacatttaaagagcacttactgtggaacaagccttcagccaaacactggagatagaaaagtaggtaagactcagcctttgtccagagaagctcagggtgtagctgaataggcagtttgttttgttctgaggaaaatcaggacatgcctgctttctaaaaatctttctctgaagacctgacccaagcccttaaatgccattgtaagagaaatttctttaagtctattaactccatttgagtagggattcactgactagattttactgaactatgaaaataaatacacataatttttcacaaaattttgggcccaattcccctaaaagaattgaggattagggagaaaggagacaactcaaagtcatcccattaagtgcagttcctttgaatcttctgctttatctttaaaaatttgtgtatatcttttattctatttattccatagatgtcttaatataaaattagtcaattcaattacaccgtcaattaaaagtaatgggcaagaggacgcatcatactaatttagtaagaacgttcctaaatgttgtaacaatgtggatcatacatctctggttttttaaatgtattgaggctttcttggtggactagtataatatacggtcagttatgtcaatgcttcatgatcaataaaaaggaagttgcaaattgt

SEQ ID NO: 32 - DNA, 3838 bp, Unknown
  PREDICTED Pongo abelii thymic stromal lymphopoietin (TSLP), mRNA.
agagtggacttttttttcaatgaatgctgtggtgacttgagtacaacacatgaagaattccagaaaagggccacttttgaagtcagcggtgaatcagaggttttccgaccctctgaatgaaagagagttcaactctggctccagtttgtcccctactcttttcccgaatccctcgacattccctgctcttttccccaggcaccgcatggcctgtgacatttgctgggactcacacacggcgtccaacctcctttctccgagcgatgactcctaggaacttcccaagggccagaacgatgcaggctccgacacgcgagggcggggggcaggggtggggggtgcgatggacgggggagcggaagggaaagagagagagaggggacctgggtgtagagagcgcccagggccgcatgagccaagctagggcggatcagggggttcatctcagcaacctgatccgtgtcggcctttccctcccttcaaccctccattggtgctgctggaactcggaagactgctagacagacatcctctagggcactctccctgactcacttggctgcgcttttaagcacccttttctggatcaacaagagtttgctagtaaaaatctgagcccgccatctcgatgcttctcctgtctctaaaactggtgcccgggctgaggccccaggtgacagacgttttccagtctccgctgcgcggggctaagaatctgggaggcagagcgcactaaagcgtgcgccgcctccgggagagctgagctcaggacagcatcctctccgtgcgctcgccgcggggcacccagcagcgcggtctggctccgttcttgtgtcgcgtcctcacgtttaacgctggccttaagagtgggactgtgaaggtggctgggagctggaatttgtggttttaaaagttcttgacacctactagaaagtgctagaggacccttcagcccctaaggtgcccctattcaccaagagtaggcgcttttcctgaaattgatatggaatatttgaaaagtagggtttttgaggacaagccacatcagatctttccccatttatttcaacctcttatcgttcctgcccctaagttctggtcacggttgcacatttaatgctttcacatgaaagtgctgtcgaagactgagactgcacccaccctccaaagcgtcccataagtaagcacagtttcttactggccaaggagaagggaaagcactagttgaaagtggccacacgaaattctctaagatgttcagaatgggcgggacatatgcaaggactccaaatccttacagtggctgctggtttcagaaggtgtttctcccaaatagaaaggattggtcatggaaatgccctgtaggagaaagacactggtatccatttcttaaaggacattcacacttatgaagtgtttagggcaaagcaaaaaggaggaagtttctttcaggaaaatcttcatcttacaacttgtagggctggtgctaacttacgacttcactaactgtgactttgagaagattaaagcagcctatctcagtactatttctaaagacctgattacatatatgagtgggaccaaaagtaccgagttcaacaacaccgtctcctgtagcaatcggccacactgccttactgaaatccagagcctaaccttcaatcccaccgccggctgcgcgtcgctcgccaaagaagtgttcgccatgaaaactaaggctgccttagctctctggtgcccaggctattcggaaactcagataaatgctactcaggcaatgaagaagaggagaaaaaggaaagtcacaaccaataaatgtctggaacaagtgtcacaattactaggattgtggcgtcgcttcaatcgacctttactgaaacaacagtaaaccatctttattatggtcatatttcacagcaccaaaataaatcatctttattaagtagatgaaacattaactctaactgtgacaaagaagaccacaaatagttatcttttaattacagaagagtttcctaacttatttttgtaagtttttattgtgtaagtttataatgcaggggaagtactactcctcaaatgttgagggaagcttccataacattgatgactggcttcatggcagtaattctcggctgtaattgcataagcattgctcaggaggaaaatccaaaagtgcagcaggagaactcttttccttggaaaaggaaaaatattgaactcaatgatagaacctaaacttacatttaaaagacagacattccttctacatgtaataacacttcttctgttaaactaaaaatttacgagagaagaaagtgaaagcaaatggggtttcacaaatagttgtaaatatagtgaagcaatctgaaataattttcaagcaaaatattgtgaaagtattctaagccaagttttaaatattatctaacagacaagagtggtatatacaagtagatcctgagaagtacctttgttacagctactataaatatacatataaattatagaatggactttaatttattttgtgaacacttttgaaaatgtacatgttcctttgtaattgacactatatatttcttaataaaataattctcaaatttgtttcttatgaattatctttcaaatctagttagacaatttgcacacatacttttgtaagggacattatctttcttcaggtttttacctccactcatccttagagcccactgactgctcccctttatacctgttggccctgcctataggagagaatatttggagataggcagcttcagaatgcattgcaatcacccttttcttaaattatgtcactagtcttttctttttttccctcttgaactttcctcacacctagaagaaacaaagtaggcaaaagtgaacaggggatgtcaaatcgattcttgaattcctgctgcaagctagaaccgcaggcaccctctcactcaacttccactcagaaccctctaaacaccagtgggaagggcaacccactgcacgtgggaatgcactgatttttcctaggagtagacatgttcctctaattactccctgagggttagttggggctaaaccatgacagaagtggggaagttcaatgtccttcaatccatcttacctgccaacaggtaagaggaagtttacattatatgtccagtccacatttaaagagcacttactgtggaacaagccttcagccaaacactggggatagcaaagtaggtaagactcagcctttgtccagagaagcttggggtgtagctgaataggcagtttcttttgtcctgaggaaaatcaggacatgcctgctttctaaaaatcttcctctgaagacctgacccaagcccttaaatgccattgtaacagaaatttctttgtctattaactccattttagtagggattcactgactagattttactgaactatgaaaataaatacacataatttttcacaaaattttgggcccaattcccctaaaagaaatgaggattagggagaaaggagacaactcaaagtcatcccattaagtgcagtttctttgaatcttctgctttatctttaataatttgtataatttatatattttattctatgtattccacagatgtcttaatgtaaaattagtcatttaaattacactgtcaattaaaagtaatgggcaagaggatgcatcatactaatttggtaagaacgttcccaaatgttgtaacaatgtggatcatacatctctggttttttaaatgtattgaggctttcttggtggactagtacagcattcggtcagttatgtcaatgtttcatggtcaataaaaaggaagctgcaaattgt

SEQ ID NO: 33 - DNA, 615 bp, Felis catus
atggccctgcaggagaaagagaggcaattcagtgtgggtggaaagactttagtgcgaaacaggggtggaactgagtttccaagtatattgcctgatgccttattatttgttctgtcagtttttttcaggaagatcttcatcttacaactggtagggctggtgctaacctacaatttcactgactgtgactttgggaagattagaaagaagtatcaggaagtcatttatcaagccctgaataaatacatgaatggggtcaagagcaccgaattcaacaactacatctactgtgaagacccgccagattgcctcgctaaaatcgacctcattaccttctatcccacccacggctgcacgacactggccaaggaaatctttgcgataagaactaacgctacgctcactctccagtgcccaggctactcaggaacgcagataaataatacccaggcaaagaagaaaaggaagaaaagacaagttacaacaaataaatgccgggaacaagtctcacacttaatggaattgtggcgtcgattcagtcgcatttcatagaaaaaaaatacaccttccttcaggctgtatttcaccccaacaaaataaatcatctttattttt

SEQ ID NO: 34 - DNA, 2629 bp, Pan troglodytes
gcagccagaaagctctggagcatcagggagactccaacttaaggcaacagcatgggtgaataagggcttcctgtggactggcaatgagaggcaaaacctggtgcttgagcagtggcccctaaggcaggccttacagatctcttacactcgtggtgggaagagtttagtgtgaaactggggtggaattgggtttccacgtatgttcccttttgccttactatatgttctgtcagtttctttcaggaaaatcttcatcttacaacttgtagggctggtgttaacttacgacttcactaactgtgactttgagaagattaaagcagcctatctcagtactatttctaaagacctgattacatatatgagtgggaccaaaagtaccgagttcaacaacaccgtctcttgtagcaatcggccacattgccttactgaaatccagagcctaaccttcaatcccaccgccggctgcgcgtcgctcgccaaagaaatgttcgccatgaaaactaaggctgccttaactatctggtgcccaggctattcggaaactcagataaatgctactcaggcaatgaagaagaggagaaaaaggaaagtcacaaccaataaatgtctggaacaagtgtcacaattacaaggattgtggcgtcgcttcaatcgacctttactgaaacaacagtaaaccatctttattatggtcatatttcacagcaccaaaataaatcatctttattaagtagatgaaacattaactctaactgtgacaaagaagaccacagatagttatcttttaattacagaagagtttcttaacttatttttgtaagtttttattgtgtaagtttataatgcaggggaagtactactcctcaaatgttgagggaagcttccataacatcgatgactgacttcatggcagtaattctcagctgtagttgcataagcattgctcaagaggaaaatccaaaagtgcagcaggagaactcttttccctgaaaaagtaaaaatattgaactcaatgatagcacctaaacttacatttaaaagacagacattccttctacatgtaatgacacttcttgtgttaaactaaaaatttacaagagaagaaagtgaaagcaaatggggtttcacaaatagttgtaaatatagtgaagcaatttgaaataattttcaagcaaaatattgtgaaagtattctaagccaagttttaaatattatctaacagacaagagtggtatatacaagtagatcctgagaagtacctttgttacagctactataaatatacatataaattatagaatctactttaatttattttgtgaacacttttgaaaatgtacatgttcctttgtaattgacactatatatttcttaataaaataattctcaaatttgtttcttatgaatcatctctcaaatctagttagacaatttgcacacatacttttctaagggacattatcttccttcaggtttttacctccactcatccttagagcccactgactgctcccctttatacctgttggccctgcctataggagagaatatttggagataggcagcttcaggatgcattgcaatcatccttttcttaaattatgtcactagtgttttattttttcccctcttgaactttcctcacacctggaagaaacaaagtaggaaaaagtgaacaggggatgtcaaatcgattcttgaattcccgctgcaagctagagccgcaggcaccctctcactcaatttccactcagaaccctataaacaccagtgggaagggcaacccactgcacgtgggaatgcactgatttttcctaggagtagacatgttcctctaattactccctgagggttagttggggctaaaccatgacagaagtggggaagttcaatgtccttaaatccatcttacttgccaacaggtaagaggaagcttacattatatgtccagtccacatttaaagagcacttactgtggaacaagccttcagccaaacaatggggatagaaaagtaggtaagactcagcctttgtccagagaagctcagggtgtagctgaataggcagtttcttttgtcctgaggaaaatcaagacatgcctgctttctaaaaatcttcctctgaagacctgacccaagcccttaaatgctattgtaagagaaatttctttgtctattaactccattttagtagggattcactgactagattttactgaactatgaaaataaatacacataatttttcacaaaattttgggcccaattcccctaaaagaattgaggattagggagaaaggagacaactcaaagtcatcccattaagtgcagtttctttgaatcttctgctttatctttaaaaattcatataatttatatattttattctatgtattccatagatatcttaatgtaaaattagtcatttaaattacactgtcaattaaaagtaatgggcaagagattgcatcatactaatttagtaagaacgttcccaaatgttgtaacaatgtggatcatacatctctggttttttaaatgtattgaggctttcttggtggactagtatagtatacggtcagttatgtcaatgtttcatggtcaataaaaaggaagttgcaaattgt

SEQ ID NO: 35 - DNA, 3834 bp, Pan paniscus
agagtggacttttttttcaaagaatgctgtggtgactcgagtacaacacatgaagaattccagaaaagagccatttttgaagtcagcggtgaatcagaggttttctgaccctctcgagtgccttctatgaagaagagttcaactctggctccagtttgtcccctactcttttcccgaatccctcgacattccttgctcttttccccaggcaccgcatggcctgtgacatttgctgggactcacacacggcgtccaacctcctttctccgagcgatgactcctaggaacttcccaaggaccggagcgatgcaggctccgacacgcgagggcggggggcgggggtgggggatgcgatggacgggggagcggaagggaaagagagagagaggggacctgggtgtagagagggcccagggccgcatgagccaagccagggaggagcagggggttcatctcagcaacctgatcggtgtcggcctttccctcccttcaaccctccattgatgttgctggaactcggaagactgctagacagacatcctctagggcactctccctgactcactttgctgtgcttttaagcactcttttctggatcaacaagagtttgctagtaaaaatctgagcccgccatctcgatgcttctcctgtctctaaaactggtgcccgggctgaggccccaggtgacagacgttttccagtctacgctgcgcggtgctaagcctctgcgaggcagagcgcactaaagcgtgcgccgcctccgggagagctgagctcaggacagcatcctctccgtgcggtcgccgctgggcacccagcagcgcggtctggctccgcttcttgtgtcgctggccttaagagtgggactgtgaaggtggctgggcgctggaatttgtggttttaaaagttcttgacacctgctagaaagtgctagaggactcttcagcccctaaggtgcccctagtcaccaagagtaggcgcttttcctgaaattgatatgggaaatttgaaaagtagggtttttgagaacaagccacatcagatctttccccacttatttcaacctcttatcgttcctgcccctaaggttctggtcacggttgcacatttaatgctttcacatggaagtgctgttgaagactgagactgcaccccaccctccaaagcatcccataagcaagcacagtttcttactagccaaggagaagggaaagcactagttgaaagtggccacaggaaatgctcaaagatgttcagaatgcatgggacatatgcaaggactccaaatccttacagtggctgctggtttcagaaggtgtttctcccaaatagaaaggattggtcacggaaatgccctgtaggagaaagacactggtatccgtttcttaaaggacattcacacttatgaagtgtttagggcaaagcaaaaaggaggaagtttctttcaggaaaatcttcatcttacaacttgtagggctggtgttaacttacgacttcactaactgtgactttgagaagattaaagcagcctatctcagtactatttctaaagacctgattacatatatgagtgggaccaaaagtaccgagttcaacaacaccgtctcttgtagcaatcggccacattgccttactgaaatccagagcctaaccttcaatcccaccgccggctgcgcgtcgctcgccaaagaaatgttcgccatgaaaactaaggctgccttagctatctggtgcccaggctattcggaaactcagataaatgctactcaggcaatgaagaagaggagaaaaaggaaagtcacaaccaataaatgtctggaacaagtgtcacaattacaaggattgtggcgtcgcttcaatcgacctttactgaaacaacagtaaaccatctttattatggtcatatttcacagcaccaaaataaatcatctttattaagtagatgaaaccttaactctaactgtgacaaagaagaccacagatagttatcttttaattacagaagagtttcttaacttatttttgtaagtttttattgtgtaagtttataatgcaggggaagtactactcctcaaatgttgagggaagcttccataacatcgatgactgacttcatggcagtaattctcagctgtagttgcataagcattgctcaagaggaaaatccaaaagtgcagcaggagaactcttttccctgaaaaaggaaaaatattgaactcaatgatagcacctaaacttacatttaaaagacagacattccttctacatgtaatgacacttcttgtgttaaactaaaaatttacaagagaagaaagtgaaagcaaatggggtttcacaaatagttgtaaatatagtgaagcaatttgaaataattttcaagcaaaatattgtgaaagtattctaagccaagttttaaatattatctaacagacaagagtggtatatacaagtagatcctgagaagtacctttgttacagctactataaatatacatataaattatagaatctactttaatttattttgtgaacacttttgaaaatgtacatgttcctttgtaattgacactatatatttcttaataaaataattctcaaatttgtttcttatgaatcatctctcaaatctagttagacaatttgcacacatacttttctaagggacattatcttccttcaggtttttacctccactcatccttagagcccactgactgctcccctttatacctgttggccctgcctataggagagaatatttggagataggcagcttcaggatgcattgcaatcatccttttcttaaattatgtcactagtgttttattttttcccctcttgaactttcctcacacctggaagaaacaaagtaggaaaaagtgaacaggggatgtcaaatcgattcttgaattcccgctgcaagctagagccgcaggcaccctctcactcaatttccactcagaaccctataaacaccagtgggaagggcaacccactgcacgtgggaatgcactgatttttcctaggagtagacatgttcctctaattactccctgagggttagttggggctaaaccatgacagaagtggggaagttcaatgtccttaaatccatcttacttgccaacaggtaagaggaagcttacattatatgtccagtccacatttaaagagcacttactgtggaacaagccttcagccaaacaatggggatagaaaagtaggtaagactcagcctttgtccagagaagctcagggtgtagctgaataggcagtttcttttgtcctgaggaaaatcaagacatgcctgctttctaaaaatcttcctctgaagacctgacccaagcccttaaatgctattgtaagagaaatttctttgcctattaactccattttagtagggattcactgactagattttactgaactatgaaaataaatacacataatttttcacaaaattttgggcccaattcccctaaaagaattgaggattagggagaaaggagacaactcaaagtcatcccattaagtgcagtttctttgaatcttctgctttatctttaaaaattcatataatttatatattttattctatgtattccatagatatcttaatgtaaaattagtcatttaaattacactgtcaattaaaagtaatgggcaagagattgcatcatactaatttagtaagaacgttcccaaatgttgtaacaatgtggatcatacatctctggttttttaaatgtattgaggctttcttggtggactagtatagtatacggtcagttatgtcaatgtttcatggtcaataaaaaggaagttgcaaattgt

SEQ ID NO: 36 - DNA, 3978 bp, Gorilla gorilla
gaagttggtgatgggggaagagtggacttttttttcaaagaatgctgtggtgactcgagtacaacacatgaagaattccagaaaagagccatttttgaagtcagccgtgaatcagaggttttctgaccctctcgagtgccttctatgaagaagagttcaactctggctccagtttgtcccctactcttttcccgaatccctcgacattccctgctcttttccccaggcaccgcatggcctgtgacatttgctgggactcacacacggcgtccaacctcctttctccgagcgatgactcctaggaacttcccaaggaccagagcgatgcaggctccgacacgcgagggcggggggcgggggtgggggggtgcgatggacgggggagcggaagggaaagagagagagaggggacctgggtgtagagagggcccagggccgcatgagccaagccagggaggagcacggggttcatctcagcaacctgatcggtgtcggcctttccctcccttcaaccctccgttggtgttgctggaactcggaagactgctagacagacatcctctagggcactctccctgactcactttgctgtgcttttaagcactcttttctggatcaacaagagtttgctagtaaaaatctgagcccgccatctcgatgcttctcctgtctctaaaactggtgcctgggctgaggccccaggtgacagacgttttccagtctacgctgcgcggggctaagcctctgggaggcagagcgcactaaagcgtgcgccgcctccgggagagctgagctcaggacagcatcctctccgtgcggtcgccgctgggcacccagcagcgcggtctggctccgcttcttgtgtcgctggccttaagagtgggactgtgaaggtggctgggcgctggaatttgtggttttaaaagttcttgacacctgctagaaagtgctagaggactcttcagcccctaaggtgcccctagtcaccaagagtaggcgcttttcctgaaattgatatgggaaatttgaaaagtagggtttttgagaacaagccatatcagatctttccccacttatttcaacctcttatcgttcctgcccctaaggttctggtcccggttgcacatttaatgctttcacatggaagtgctgtcgaagactgagactgcacccaccctccaaagcatcccataagcaagcacagtttcttactagccaaggagaagggaaagcactagttgaaagtggccacacgaaatgctcaaagatgttcagaatgcatgggacatatgcaaggactccaaatccttacagtggctgctggtttcagaaggtgtttctcccaaatagaaaggattggtcacggaaatgccctgtaggagaaagacacttgtatccatttcttaaaggacattcacacttctgaagtgtttagggcaaagcaaaaaggaggaagtttctttcaggaaaatcttcatcttacaacttgtagggctggtgttaacttacgacttcactaactgtgactttgagaagattaaagcagcctatctcagtactatttctaaagacctgattacatatatgagtgggaccaaaagtaccgagttcaacaacactgtctcctgtagcaataggccacactgccttactgaaatccagagcctaaccttcaatcccaccgccggctgcgcgtcgctcgccaaagaaatgttcgccatgaaaactaaggctgccttagctatctggtgcccaggctattcggaaactcagataaatgctactcaggcaatgaagaagaggagaaaaaggaaagtcacaaccaataaatgtctggaacaagtgtcacaattacaaggattgtggcgtcgcttcaatcgacctttactgaaacaacagtaaaccatctttattatggtcatatttcacagcaccaaaataaatcatctttattaagtagatgaaacattaactctaactgtgacaaagaagaccacaaatagttatcttttaattacagaagagtttcttaacttatttttgtaagtttttattgtgtaagtttataatgcaggggaagtactactcctcaaatgttgagggaaggttccataacattgatgactggcttcatggcagtaattctcggctgtagttgcataagcattgctcaagaggaaaatccaaaagtgcagcagaactcttttccctgaaaaaggaaaaatattgaactcaatgatagcacctaaacttacatttaaaagacagacattccttctacatgtaatgacacttcttgtgttaaactaaaaatttacaagagaagaaagtgaaagcaaatggggtttcacaaatagttgtaaatatagcgaagcaatttgaaataattttcaagcaaaatattgtgaaagtattctaagccaagttttaaatattatctaacagacaagagtggtatatacaagtagatcctgagaagtacctttgttacagctactataaatatacatataaattatagaatctactttaatttattttgtgaacacttttgaaaatgtacatgttcctttgtaattgacactatatatttcttaataaaataattctcaaatttgtttcttatgaatcatctctcaaatctagttagacaatttgcacacatacttttctaagggacattatcttccttcaggtttttacctccactcatccttagagcccactgactgctcccctttatacctgttggccctgcctataggagagaatatttggagataggcagcttcaggatgcattgcaatcatccttttcttaaattatgtcactagtcttttattttttcccctcttgaactttcctcacacccggaagaaacaaagtaggcaaaagtgaacaggggatgtcaaatcgattcttgaattcccgctgcaagctacagccgcaagcaccctctcactcaacttccactcagaaccctataaacaccagagggaagggcaacccactgcacgtgggaatgcactgatttttcctaggagtagacatgttcctctaattactccctgagggttagttggggctaaaccgtgacagaagtggggaagttcaatgtccttaaatccatcttacttgccaacaggtaagaggaagcttacattatatgtccagtccacatttaaagagcacttactgtggaacaagctttcagccaaacaatggggatagaaaagtaggtaagactcagcctttgtccagagaagctcagggtgtagctgaataggcagtttcttttgtcctgaggaaaatcaggacatgcctgctttctaaaaatcttcctctgaagacctgacccaagcccttaaatgctattgtaagagaaatttctttgtctattaactccattttagtagggattcactgactagattttactgaactatgaaaataaatacacataatttttcacaaaattttgggcccaattcccctaaaagaattgaggattagggagaaaggagacaactcaaagtcatcccattaagtgcagtttctttgaatcttctgctttatctttaaaaatttgtataatttatatattttattcgatgtattccatagatatcttaatgtaaaattagtcatttgaattacactgtcaattaaaagtaatgggcaagagattgcatcatactaatttagtaagaacgttcccaaatgttgtaacaacgtggatcatacatctctggttttttaaatgtattgaggctttcttggtggactagtatagtatacggtcagttatgtcaatgtttcatgatcaataaaaaggaggttgcaaattgtgatttaagcatataaagttggttatatacatactgagactacattgttagttatgctatttatataatctatattctcacttttgtctccttcattcagaggctaagagagctgtatgaaagtctactg

SEQ ID NO: 37 - DNA, 465 bp, Canis lupus
ttgcctgatgccttattatctgttctgtcagtgtttttcaggaagatcttcgtcttgcagctggtagggctggtgctaacctacaatttcattgactgtgactttgagaagattagatggaagtatcaggaagtcatttaccaagccctggagaaatacatggatgggaccaggagcacggagttcagccaccccgtgtactgcgcggacccgcccgactgcctggccaggatcgagcggctcaccctgcaccgcatccgcggctgcgcgtcgggcgcccgggaggccttcgccgaggggacggtcgccgcgctcgccgccgagtgcccgggctacgccgcagcgccgataaataatacccaggcaaagaagaaaagaaaaaaaagaggagtcacaacaaataaatgccgggaacaagtcgcacacttaatagggctgtggcgtcgtttcagtcgcatttcatag

SEQ ID NO: 38 - DNA, 447 bp, Ailuropoda melanoleuca
tttgttctgtcagtttttttcaggaaaatcttcatcttgcaactggtagggctggtgctaacctacaattttactggctgtgactttgagaagattagaaagaagtatcaggaagtcatttatcaagccctggagaagtacatgaatgggaccaaaagcaccgaattcaacaaccccatctactgtgaagacccgccagactgcctcgctaaaatcgaacgcattaccttccatcccacccacgactgcacgtctctcgccgaggaaatctttgcaataggaactagggctacgctcactctccagtgccccagctactccggaatgcagataaataatacccaggcaaagaagaaaagaaagaaaagaaaagtcacaacaaataaatgccgggaacaagttgcatacttaataggactgtggcgtcgtttcagtcgcatttcatag

SEQ ID NO: 39 - DNA, 1143 bp, Mus musculus
cacgttcaggcgacagcatggttcttctcaggagcctcttcatcctgcaagtactagtacggatggggctaacttacaacttttctaactgcaacttcacgtcaattacgaaaatatattgtaacataatttttcatgacctgactggagatttgaaaggggctaagttcgagcaaatcgaggactgtgagagcaagccagcttgtctcctgaaaatcgagtactatactctcaatcctatccctggctgcccttcactccccgacaaaacatttgcccggagaacaagagaagccctcaatgaccactgcccaggctaccctgaaactgagagaaatgacggtactcaggaaatggcacaagaagtccaaaacatctgcctgaatcaaacctcacaaattctaagattgtggtattccttcatgcaatctccagaataaaattagctttcagcttctgctatgaaaatctctatcttggttttagtggacagaatactaagggtgtgacacttagaggaccactggtgtttattctttaattacagaagggattcttaacttattttttggcatatcgcttttttcagtataggtgctttaaatgggaaatgagcaatagaccgttaatggaaatatctgtactgttaatgaccagcttctgagaagtctttctcacctcccctgcacacaccttactctagggcaaacctaactgtagtaggaagagaattgaaagtagaaaaaaaaaattaaaaccaatgacagcatctaaaccctgtttaaaaggcaaggatttttctacctgtaatgattcttctaacattcctatgctaagattttaccaaagaagaaaatgacagttcgggcagtcactgccatgatgaggtggtctgaaagaagattgtggaatctgggagaaactgctgagatcatattgcaaatccagctgtcaaagggttcagacccaggacagtacaattcgtgagcagatctcaagagccttgcacatctacgagatatatatttaaagttgtagataatgaatttctaatttattttgtgagcacttttggaaatatacatgctactttgtaatgaatacatttctgaataaagtaattctcaagtttgaaaaaaaaaa

(19) United States
(12) Patent Application Publication
(10) Pub. No.: US 2016/0052985 A1
(43) Pub. Date: Feb. 25, 2016

(54) THYMIC STROMAL LYMPHOPOIETIN FRAGMENTS AND USES THEREOF

(71) Applicant: IEO - ISTITUTO EUROPEO DI ONCOLOGIA SRL, Milano (MI) (IT)

(72) Inventors: Maria RESCIGNO, Milano (MI) (IT); Giuseppe PENNA, Milano (MI) (IT); Katerina TSILINGIRI, Milano (MI) (IT); Giulia FORNASA, Milano (MI) (IT)

(21) Appl. No.: 14/781,866

(22) PCT Filed: Apr. 4, 2014
(86) PCT No.: PCT/EP2014/056878

§ 371 Date: Oct. 1, 2015

(30) Foreign Application Priority Data
Apr. 4, 2013 (EP) 13162326.6

(51) Int. Cl. C07K 14/52; C12Q 1/68; C07K 16/24

(52) U.S. Cl. CPC C07K 14/52; C07K 16/24; C12Q 1/6883; A61K 38/00

(57) ABSTRACT

The present invention relates to a compound selected in the group consisting of: a) a polypeptide consisting of the C-terminus amino acid sequence of the human Thymic stromal lymphopoietin (TSLP) long isoform (SEQ ID NO: 2), derivatives and fragments thereof; b) a polynucleotide coding for said polypeptide; d) a vector comprising said polynucleotide; c) a host cell genetically engineered expressing said polypeptide, or d) a TSLP long isoform (SEQ ID NO:2) antagonist for use in the treatment or/and prevention and/or diagnosis and/or monitoring of a disorder or pathology characterized by an inflammatory response.

### FIELD OF THE INVENTION

The present invention relates to fragments of the long isoform of thymic stromal lymphopoietin (TSLP) and their use as an anti-inflammatory and/or homeostatic agent.

### BACKGROUND OF THE INVENTION

Thymic stromal lymphopoietin (TSLP) is a cytokine involved in several physiological immune activities. TSLP plays a major role in several homeostatic immune responses.

It is expressed by the Hassal's corpuscles in the thymus and regulates the capacity of dendritic cells (DCs) (Watanabe et al., 2005) and plasmacytoid DCs (Hanabuchi et al., 2010) to drive the development of natural T regulatory (Treg) cells. TSLP can also promote the homeostatic polyclonal expansion of T cells in the absence of foreign antigen (Watanabe et al., 2004). In the gut, it is expressed by intestinal epithelial cells and educates non-inflammatory DCs that have reduced ability to produce IL-12p70 (Rimoldi et al., 2005) and drive the differentiation of inducible Tregs (Iliev et al., 2009). It is also expressed by DCs in response to Toll-like receptor agonists (Kashyap et al., 2011; Spadoni et al., 2012) and at steady-state by intestinal CD103+ DCs (Spadoni et al., 2012). DC-derived TSLP in the mouse system acts directly on T cells by limiting their differentiation towards Th17 and by fostering Treg development (Spadoni et al., 2012). Consistently, absence of TSLP or TSLP signaling leads to an increased susceptibility to Dextran sodium sulphate (DSS)-induced colitis (Reardon et al., 2011; Spadoni et al., 2012; Taylor et al., 2009) presumably via a direct action on T cells (Spadoni et al., 2012) or through the release by epithelial cells of secretory leukocyte peptidase inhibitor (SLPI), an endogenous inhibitor of neutrophil elastase that helps reducing inflammation (Reardon et al., 2011). Of note, TSLP expression is dependent on the microbiota and in particular on nucleic acids, via the IRF3 transcription factor (Negishi et al., 2012). This may explain the prompt release of TSLP in response to viral infections (Fontenot et al., 2009) and double stranded RNA (Kinoshita et al., 2009), that lead to the activation of virus-specific CD8+ effector T cells (Yadava et al., 2013). TSLP is required for the induction of Th2 type of responses that are physiologically relevant for the elimination of parasites (Zaph et al., 2007). Conversely, TSLP has been shown to play a pathogenic role in several immune disorders. Indeed, TSLP can drive the development of strong allergic Th2 responses with the release of IL-4, IL-5, IL-13 and TNF-a (Soumelis et al., 2002) via the upregulation of OX-40 ligand expression on TSLP-treated DCs (Ito et al., 2005). TSLP is overexpressed in the airways of asthmatic patients and is associated to Th2 cytokines (Ferreira et al., 2012; Ying et al., 2005). Consistently, TSLP polymorphisms are linked to asthma susceptibility (Harada et al., 2011; Hunninghake et al., 2010; Liu et al., 2012) and TSLP has been shown to participate to asthma development in mouse models (Al-Shami et al., 2005; Zhou et al., 2005). TSLP is also directly involved in the differentiation of basophils that are more prone to promote Th2 inflammation (Siracusa et al., 2011). Accordingly, TSLP is involved in other allergic disorders, including atopic dermatitis and food allergy (Ziegler, 2012).

Hence, apparently TSLP has both physiological and pathological activities that are difficult to be reconciled. TSLP is found in two different isoforms in human, the “long” isoform (lTSLP) and the “short” isoform (sTSLP or shTSLP), each driven by an independent promoter (UCSC genome browser).

Xie Y, et al (J. of Dermatological Science, June 2012; 66 (3): 233-237.) compared the expression of the long-form and total TSLP transcripts in primary human keratinocytes; in particular, they showed that TLR ligands, proinflammatory and Th2 cytokines upregulated long-form TSLP gene expression, but not the short-form, indicating that the long TSLP contributes to the production of TSLP protein under inflammatory conditions. Without stimulation, the short-form was constitutively expressed or further upregulated in culture conditions with overgrowth or with VDR (Vitamin D receptor) agonists (i.e. Calcitriol). The two following studies (Di Piazza et al; Demehri et al) demonstrate that TSLP-mediated inflammation can be tumour suppressive in some mouse models of skin cancer. TSLP signalling in cancer is context-dependent, then caution in the current development of TSLP-inactivating therapeutic agents is suggested.

Di Piazza M, et al. (Cancer cell. 2012 October 16; 22(4):479-93.) has addressed the role of TSLP during skin carcinogenesis. They demonstrated that TSLP-mediated inflammation protects against cutaneous carcinogenesis by acting directly on CD4 and CD8 T cells. In addition, they showed that TSLP can induce antitumorigenic inflammation by acting directly on T cells, which prevents the growth of beta-catenin-dependent skin tumours. Demehri S, et al. (Cancer cell. 2012 Oct. 16; 22(4):494-505) demonstrated that TSLP triggers a dominant antitumor response in Th2-polarized inflammatory microenvironment in the skin. In particular, they showed that high levels of TSLP released by barrier-defective skin caused a severe inflammation, resulting in gradual elimination of Notch-deficient epidermal clones and resistance to skin tumorigenesis.

Neither Di Piazza M, et al. nor Demehri S, et al. mentioned a short isoform of TSLP nor an anti-inflammatory role of TSLP.

Harada M, et al. (American J of Respir Cell Mol Biol 2009; 40:368-74.) refers to two TSLP splicing variants, a short one and a long one, but they do not describe a potential anti-inflammatory role of the short one. He J Q, et al. (J Allergy Clin Immunol 2009) described gene variants (different polymorphisms) and not splicing variants of TSLP. Taylor B C, et al. (J Exp Med 2009) showed that TSLP-TSLPR interactions are critical for immunity to the intestinal pathogen Trichuris. TSLPR(−/−) mice displayed elevated production of IL-12/23p40 and IFN-gamma, and developed heightened intestinal inflammation upon exposure to dextran sodium sulfate, demonstrating a previously unrecognized immune-regulatory role for TSLP in a mouse model of inflammatory bowel disease. They suggested that a function of TSLP in the intestinal microenvironment may be to either directly or indirectly inhibit proinflammatory cytokine production and help prevent the development of severe intestinal inflammation. This study does not mention different isoforms of TSLP.

The patent application US2010/0021486 relates to compositions for the treatment of Th2 mediated inflammatory conditions. The compositions comprise a non-primate TSLP or fragments thereof to be used as vaccines to treat humans with excessive mediated inflammatory conditions. For the treatment of Th2 mediated inflammatory conditions in other non-human mammals, the TSLP of the species to be vaccinated is being used as antigen.

The patent application WO2005007186 concerns methods of treatment for tumors, in particular by administering TSLP and diagnosis methods of neoplasms. The diagnostic method comprises incubating a sample with an anti-TSLP or anti-TSLP-receptor and detecting the formation of an antibody-antigen complex. The neoplasm is an epithelial derived cancerous tumor that may be a breast tumor, colon tumor, lung tumor, ovarian tumor, or a prostate tumor.

TW201206471 refers to compositions and methods for an immunotherapeutic approach for human breast cancer. Any antagonist of thymic stromal lymphopoietin (TSLP) and/or OX40L to inhibit tumor development and IL-13 secretion by blocking the upregulation of OX40L by DCs exposed to breast cancer, thereby blocking their capacity to generate inflammatory IL-13+ TNF [alpha]+ IL-10neg CD4+ T cells (Th2 cells). In particular it refers to a therapeutic composition for the treatment of a tumor of epithelial origin in a human subject comprising one or more active agents that bind and neutralize the activity of a thymic stromal lymphopoietin (TSLP), wherein the one or more active agents are selected from the group consisting of an anti-TSLP antibody; an anti-TSLP antibody fragment; an anti-TSLP antibody-carrier conjugate; a TSLP binding fusion protein; a TSLP antagonist; a TSLP inhibitor a TSLP receptor antagonist; or a TSLP blocking agent optionally solubilized, dispersed or suspended in a suitable medium in an amount sufficient to inhibit development of the tumor.

US2012020988 refers to an antibody specifically binding to human thymic stromal lymphopoietin receptor (TSLPR), useful for the treatment of immunological diseases.

Up to now, it is difficult to assess the activity of each isoform due to lack of reagents. Therefore, it is still felt the need to find tools to investigate the function of the two isoforms of TSLP and to explain the paradoxical roles of TSLP, in order to provide new therapeutic agents or diagnostic biomarkers.

### DESCRIPTION OF THE INVENTION

The present inventors herein cloned and expressed each isoform of TSLP in baculovirus and surprisingly found that the two different isoforms elicit distinct immunological responses: the long isoform drives a Th2 response, while the short isoform is strongly anti-inflammatory as it inhibits the ability of DCs to respond to bacteria and to produce inflammatory cytokines such as IL-12p70, TNF-a and IL-6. As a consequence, short TSLP-conditioned DCs inhibits Th1 T cell differentiation. They found that the short isoform is the only one expressed by intestinal epithelial cells under steady-state conditions and they propose that the homeostatic activities of TSLP are mediated by the short isoform and this may explain the paradoxical roles of TSLP.

### DETAILED DESCRIPTION OF THE INVENTION

The inventors have identified several effectors mediating the control of the development of inflammatory responses and found that one in particular is very interesting. This is a cytokine called thymic stromal lymphopoietin (TSLP) that is produced by epithelial cells.

In humans, two homologous isoforms of TSLP are expressed, the “long” isoform (159 AA, SEQ ID NO: 2) and the “short” isoform (63 AA, SEQ ID NO: 1). Even though the most well characterized form of TSLP so far is the long one, the authors found the putative promoter for the long isoform to be almost completely inactive in most of the cell lines present in the UCSC database (http://genome.ucsc.edu, in particular for the TSLP: http://genome-euro.ucsc.edu/cgi-bin/hgTracks?db=hg19&position=chr5%3A110404856-110414355&hgsid=197243096_TsWaq2VphlzNP2DIjsuWKs3iDOt3). On the contrary, the promoter region for the short isoform seemed to have a high capacity to bind a number of different transcription factors and the protein is readily expressed in various tissues.

Based on this preliminary data, the authors cloned and expressed each isoform in baculovirus and showed that the two different isoforms elicit distinct immunological responses. The long isoform drives a Th2 response, while the short isoform is strongly anti-inflammatory as it inhibits the ability of monocyte-derived DCs to respond to bacteria and to produce inflammatory cytokines such as IL-12p70, IL1b, TNF-a and IL-6. The authors found that the short isoform is the only one expressed by intestinal epithelial cells under steady-state conditions. The long isoform instead shows an opposite trend, in fact it is expressed by epithelial cells only in inflamed tissue. The inventors propose that the homeostatic activities of TSLP are mediated by the short isoform and this may explain the paradoxical roles of TSLP.

The authors found that in the presence of short TSLP, IFN-gamma (here indicated also as IFNg or IFNγ) production is significantly down-regulated in a dose dependent fashion in mixed lymphocyte reactions of peripheral blood mononuclear cells. The long isoform caused instead the up-regulation of IFN-gamma. In addition, monocyte derived dendritic cells conditioned with the short isoform presented a diminished inflammatory potential after bacterial challenge as attested by cytokine secretion profiling. Further, a long form specific antibody was obtained and used to monitor expression of the two isoforms by immunofluorescence on healthy and IBD tissues at the protein level. The authors confirmed the data previously mentioned which had been obtained by real time PCR.

The authors have generated all the tools to study the function of the two isoforms of TSLP. Indeed, the fragment of long TSLP that is absent in the short isoform (aa. 1-96 of SEQ ID NO: 2) was cloned and expressed in E. coli with a DsbA tag. The resulting protein was used to immunize 2 rabbits in the presence of Freund's incomplete adjuvant. The sera of the animals were obtained and tested for their specificity against both isoforms and DsbA. Anti-DsbA antibodies were removed with DsbA-loaded columns and the resulting preparations were once again tested. Polyclonal antibody that recognized the long isoform, but not the short custom-synthesized peptide, was purified from the serum of the rabbit by HPLC. Finally, real time PCR also confirmed differential expression with similar trends in untreated coeliac disease patients and between healthy skin and several pathologic conditions like psoriasis, atopic dermatitis, sarcoidosis and mycosis fungoides. Differential expression of the two isoforms has been observed also between healthy skin and skins from condylomata accuminata, lichen ruber, basal cell carcinoma, actinic keratosis, lupus erythematodes and carcinoma. TSLP short and long isoforms are both downregulated in neoplastic tissue of colon cancer patients. The authors have found that also immune cells like dendritic cells express TSLP. In IBD lTSLP is drastically upregulated by the recruited immune cells. Hence, the inventors have hypothesized and confirmed that the two isoforms had different activities, with the sTSLP being anti-inflammatory and lTSLP being inflammatory.

The inventors propose the use of short TSLP as an anti-inflammatory agent to be administered systemically, orally or topically.

It is therefore an object of the invention a compound selected in the group consisting of:

a) a polypeptide being a fragment of the C-terminus amino acid sequence of the human Thymic stromal lymphopoietin (TSLP) long isoform (SEQ ID NO: 2), or a fragment of the corresponding sequence encoded from a TSLP orthologous or homologous gene, functional mutants, recombinant or synthetic derivatives, fragments or analogues thereof;
b) a polynucleotide coding for said polypeptide;
c) a vector comprising said polynucleotide;
d) a host cell genetically engineered expressing said polypeptide.

Preferably, said C-terminus amino acid sequence of the TSLP long isoform (SEQ ID NO: 2) consists of the amino acid sequence of the TSLP short isoform (SEQ ID NO: 1), or of an amino acid sequence of the corresponding region encoded from a TSLP orthologous or homologous gene, functional mutants, recombinant or synthetic derivatives, fragments or analogues thereof.

More preferably, the polypeptide as above defined is comprised in a sequence consisting essentially of the aa. 4-63 of the amino acid sequence of the TSLP short isoform (SEQ ID NO: 1), or in an amino acid sequence of the corresponding region encoded from a TSLP orthologous or homologous gene, functional mutants, recombinant or synthetic derivatives, fragments or analogues thereof.

In a preferred aspect, the polypeptide as above defined comprises a sequence consisting essentially of the aa. 4-40 or aa. 24-63 of the sequence of the TSLP short isoform (SEQ ID NO: 1) or of an amino acid sequence of the corresponding region encoded from a TSLP orthologous or homologous gene, functional mutants, recombinant or synthetic derivatives, fragments or analogues thereof.

Preferably, the polypeptide as above defined consists of an amino acid sequence consisting essentially of aa. 4-40 or aa. 24-63 of the sequence of the TSLP short isoform (SEQ ID NO: 1), or of the an amino acid sequence of the corresponding region encoded from a TSLP orthologous or homologous gene, functional mutants, recombinant or synthetic derivatives, fragments or analogues thereof.

In another preferred embodiment of the invention, the polypeptide consists essentially of the aa. 4-63 of the amino acid sequence of the TSLP short isoform (SEQ ID NO: 1), or of an amino acid sequence of the corresponding region encoded from a TSLP orthologous or homologous gene, functional mutants, recombinant or synthetic derivatives, fragments or analogues thereof. The compound as above defined preferably has an immunomodulatory activity, more preferably said immunomodulatory activity is an anti-inflammatory activity.

The compound as above defined may be used as a biomarker.

Another object of the invention is the compound as above defined for medical use, preferably for use in the treatment and/or prevention of a disorder or pathology characterized by an inflammatory response, and/or for use in a method for the diagnosis and/or prognosis of a disorder or pathology characterized by an inflammatory response and/or for the monitoring of disorder or pathology characterized by an inflammatory response and/or for the monitoring the efficacy of a therapeutic treatment of a disorder or pathology characterized by an inflammatory response and/or for the screening of a therapeutic treatment of a disorder or pathology characterized by an inflammatory response.

The compound as above defined may be used as a biomarker, in particular in the methods as above defined.

A further object of the invention is a compound selected in the group consisting of:

a) a polypeptide consisting of the C-terminus amino acid sequence of the human Thymic stromal lymphopoietin (TSLP) long isoform (SEQ ID NO: 2), or a polypeptide consisting of the corresponding sequence encoded from a TSLP orthologous or homologous gene, functional mutants, recombinant or synthetic derivatives, fragments or analogues thereof;
b) a polynucleotide coding for said polypeptide;
c) a vector comprising said polynucleotide;
d) a host cell genetically engineered expressing said polypeptide
for use in the treatment and/or prevention of a disorder or pathology characterized by an inflammatory response.

It is another object of the invention, a compound selected in the group consisting of:

a) a polypeptide consisting of the C-terminus amino acid sequence of the human Thymic stromal lymphopoietin (TSLP) long isoform (SEQ ID NO: 2), or a polypeptide consisting of the corresponding sequence encoded from a TSLP orthologous or homologous gene, functional mutants, recombinant or synthetic derivatives, fragments or analogues thereof and/or
a polypeptide consisting of the human Thymic stromal lymphopoietin (TSLP) long isoform (SEQ ID NO: 2), or a polypeptide consisting of the corresponding sequence encoded from a TSLP orthologous or homologous gene, functional mutants, recombinant or synthetic derivatives, fragments or analogues thereof;
b) a polynucleotide coding for said polypeptide;
c) an antibody or fragment thereof capable of binding selectively to at least one of said polypeptide;
d) primer and/or probes specific for said polynucleotide;
for use in a method for the diagnosis and/or prognosis of a disorder or pathology characterized by an inflammatory response,
and/or for the monitoring of disorder or pathology characterized by an inflammatory response,
and/or for the monitoring the efficacy of a therapeutic treatment of disorder or pathology characterized by an inflammatory response,
and/or for the screening of a therapeutic treatment of a disorder or pathology characterized by an inflammatory response.

Preferably, in the compound as above defined the C-terminus amino acid sequence of the TSLP long isoform (SEQ ID NO: 2) consists of the amino acid sequence of the TSLP short isoform (SEQ ID NO: 1), or of an amino acid sequence of the corresponding region encoded from a TSLP orthologous or homologous gene, functional mutants, recombinant or synthetic or derivatives, fragments or analogues thereof.

Preferably, the above derivatives are selected from the group comprising polypeptides having a percentage of identity of at least 41%, preferably at least 75%, more preferably of at least 85% with SEQ ID NO:1 or with an amino acid sequence of the corresponding region encoded from a TSLP orthologous or homologous gene.

In a preferred aspect, the compound as above defined or the compound for use as above defined is a synthetic derivative of the polypeptide.

Preferably, said fragment refers to polypeptide having a length of at least 10 amino acids, preferably at least 17, more preferably at least 20, even more preferably at least 37 or 40 amino acids.

In the compound as above defined or in the compound for use as above defined, the polynucleotide is preferably selected in the group consisting of RNA or DNA, more preferably said polynucleotide is DNA and/or said polynucleotide comprises a sequence which encodes the sequence SEQ ID NO:1.

In the compound as above defined or in the compound for use as above defined, the vector is preferably an expression vector, more preferably selected in the group consisting of: plasmids, viral particles and phages.

In the compound as above defined or in the compound for use as above defined, the host cell is preferably selected in the group consisting of: bacterial cells, fungal cells, insect cells, animal cells, and plant cells, preferably said host cells is an animal cell.

A further object of the invention is a compound being a TSLP long isoform (SEQ ID NO:2) antagonist wherein said antagonist is:
an antibody or antibody fragment capable of binding selectively to the TSLP long isoform (SEQ ID NO:2) or to fragments thereof and of neutralizing the activity of the TSLP long isoform,
an antibody or antibody fragment capable of binding selectively to the TSLP long isoform (SEQ ID NO:2) receptor; or
an isolated TSLP long isoform receptor,
   for use in the treatment or/and prevention of a disorder or pathology characterized by an inflammatory response.

Said antibody is preferably a monoclonal or polyclonal antibody, or synthetic or recombinant derivatives thereof, more preferably said antibody being a humanized monoclonal antibody.

In a preferred embodiment a human or animal is affected by the disorder or the pathology.

Another object of the invention is a pharmaceutical composition comprising at least one pharmaceutically acceptable excipient and a compound as above defined, optionally further comprising at least one immunomodulatory agent, wherein said immunomodulatory agent is preferably an anti-inflammatory agent.

A further object is a pharmaceutical composition for use in the treatment or/and prevention of a disorder or pathology characterized by an inflammatory response comprising a compound as above defined and at least one pharmaceutically acceptable excipient, optionally further comprising at least one immunomodulatory agent, wherein said immunomodulatory agent is preferably an anti-inflammatory agent.

According to the present invention, the at least one immunomodulatory agent may be a corticosteroid (i.e. beclomethasone/beclometasone, budesonide, flunisolide, fluticasone propionate, triamcinolone, Methylprednisolone, Prednisolone/prednisolon, Prednisone, etc), non-steroid (i.e. aspirin, ibuprofen and naproxen, etc) anti-inflammatory agents, Vitamin D3, etc.

The above pharmaceutical compositions are preferably for systemic, oral, locally, preferably rectally, or topical administration.

Another object of the invention is a method for the diagnosis and/or prognosis of a disorder or pathology characterized by an inflammatory response and/or for the monitoring of disorder or pathology characterized by an inflammatory response, and/or for the monitoring the efficacy of a therapeutic treatment of a disorder or pathology characterized by an inflammatory response and/or for the screening of a therapeutic treatment of a disorder or pathology characterized by an inflammatory response in a subject comprising the steps of:
measuring the amount of at least one polypeptide consisting of:
   a) the C-terminus amino acid sequence of the human TSLP long isoform (SEQ ID NO: 2), or the corresponding sequence encoded from a TSLP orthologous or homologous gene, functional mutants, recombinant derivatives, fragments or analogues thereof and/or
   b) the human TSLP long isoform (SEQ ID NO: 2) or the corresponding protein encoded from a TSLP orthologous or homologous gene, functional mutants, recombinant derivatives, fragments or analogues thereof
   or measuring the amount of at least one polynucleotide coding for one or more of said polypeptide
   in an isolated biological sample obtained from a subject and
comparing the same with a value from a control sample.

In a preferred aspect, the method of the invention further comprises the step of calculating the ratio between the amount of a) and b) according to the following formula: amount of a)/amount of b).

Preferably, the step of measuring the amount of a) and/or b) comprises:
contacting the biological sample obtained from the subject with at least one antibody capable of binding selectively to:
   a) the C-terminus amino acid sequence of the human TSLP long isoform (SEQ ID NO: 2), or the corresponding sequence encoded from a TSLP orthologous or homologous gene, functional mutants, recombinant derivatives, fragments or analogues thereof and/or
   b) the human TSLP long isoform (SEQ ID NO: 2) or the corresponding protein encoded from a TSLP orthologous or homologous gene, functional mutants, recombinant derivatives, fragments or analogues thereof,
   under conditions for the formation of an antibody-antigen complex and,
detecting said complex.

Another object of the invention is a kit for the diagnosis and/or prognosis of a disorder or pathology characterized by an inflammatory response, and/or for the monitoring of disorder or pathology characterized by an inflammatory response, and/or for the monitoring the efficacy of a therapeutic treatment of a disorder or pathology characterized by an inflammatory response and/or for the screening of a therapeutic treatment of a disorder or pathology characterized by an inflammatory response, comprising:
means to measure the amount of at least one polypeptide consisting of:
   a) the C-terminus amino acid sequence of the human TSLP long isoform (SEQ ID NO: 2), or the corresponding sequence encoded from a TSLP orthologous or homologous gene, functional mutants, recombinant derivatives, fragments or analogues thereof and/or
   b) the human TSLP long isoform (SEQ ID NO: 2) or the corresponding protein encoded from a TSLP orthologous or homologous gene, functional mutants, recombinant derivatives, fragments or analogues thereof,
   and/or means to measure the amount of at least one polynucleotide coding for one or more of said polypeptide
   and optionally,
control means.

Control means can be used to compare the amount or the increase of amount of the compound as above defined to a value from a control sample. The value may be obtained for example, with reference to known standard, either from a normal subject or from normal population.

The means to measure the amount of at least one compound as above defined are preferably at least one antibody, functional analogous or derivatives thereof. Said antibody, functional analogous or derivatives thereof are specific for said compound.

In a preferred embodiment, the kit of the invention comprises:
a solid phase adhered antibody specific for said compound;
detection means of the ligand specific-biomarker complex.

The kits according to the invention can further comprise customary auxiliaries, such as buffers, carriers, markers, etc. and/or instructions for use.

In the case of a method or a kit for the diagnosis and/or prognosis of a disorder or pathology characterized by an inflammatory response, the value from a control may be the value measured in a sample taken from a healthy patient or from a patient affected by another disorder or pathology not characterized by an inflammatory response. In the case of a method or a kit for monitoring of disorder or pathology characterized by an inflammatory response, the progress of the disorder or pathology is monitored and the value from a control sample may by a value measured in a sample taken from the same subject at various times or from another patient. In the case of a method or a kit for monitoring the efficacy of a therapeutic treatment, the value from a control sample may by a value measured in a sample taken from the same subject before initiation of the therapy or taken at various times during the course of the therapy. In the case of a method or a kit for the screening of a therapeutic treatment of a disorder or pathology characterized by an inflammatory response, the value from a control sample may be the average of the values measured in samples taken from subjects without treatment and from subjects treated with a substance that is to be assayed or from subjects treated with a reference treatment.

In the present invention, the expression “measuring the amount” can be intended as measuring the amount or concentration or level of the respective protein and/or mRNA thereof and/or DNA thereof, preferably semi-quantitative or quantitative. Measurement of a protein can be performed directly or indirectly. Direct measurement refers to the amount or concentration measure of the biomarker, based on a signal obtained directly from the protein, and which is directly correlated with the number of protein molecules present in the sample. This signal—which can also be referred to as intensity signal—can be obtained, for example, by measuring an intensity value of a chemical or physical property of the biomarker. Indirect measurements include the measurement obtained from a secondary component (e.g., a different component from the gene expression product) and a biological measurement system (e.g. the measurement of cellular responses, ligands, “tags” or enzymatic reaction products).

The term “amount”, as used in the description refers but is not limited to the absolute or relative amount of proteins and/or mRNA thereof and/or DNA thereof, and any other value or parameter associated with the same or which may result from these. Such values or parameters comprise intensity values of the signal obtained from either physical or chemical properties of the protein, obtained by direct measurement, for example, intensity values in an immunoassay, mass spectroscopy or a nuclear magnetic resonance. Additionally, these values or parameters include those obtained by indirect measurement, for example, any of the measurement systems described herein. Methods of measuring mRNA and DNA in samples are known in the art. To measure nucleic acid levels, the cells in a test sample can be lysed, and the levels of mRNA in the lysates or in RNA purified or semi-purified from lysates can be measured by any variety of methods familiar to those in the art. Such methods include hybridization assays using detectably labeled DNA or RNA probes (i.e., Northern blotting) or quantitative or semi-quantitative RT-PCR methodologies using appropriate oligonucleotide primers. Alternatively, quantitative or semi-quantitative in situ hybridization assays can be carried out using, for example, tissue sections, or unlysed cell suspensions, and detectably labeled (e.g., fluorescent, or enzyme-labeled) DNA or RNA probes. Additional methods for quantifying mRNA include RNA protection assay (RPA), cDNA and oligonucleotide microarrays, representation difference analysis (RDA), differential display, EST sequence analysis, and serial analysis of gene expression (SAGE).

If by comparing the measured amount of the compound as above defined in b) (i.e. the human TSLP long isoform (SEQ ID NO: 2) or the corresponding protein encoded from a TSLP orthologous or homologous gene, functional mutants, recombinant derivatives, fragments or analogues thereof) with the value obtained from a control sample, the amount of said compound in the test sample isolated from the subject corresponds to a higher value, the subject may present the disorder or pathology characterized by an inflammatory response or go towards an aggravation of the said disorder or pathology.

If by comparing the measured amount of the compound as above defined in b) with the value obtained from a control sample, the amount of said compound in the test sample isolated from the subject corresponds to a similar or lower value, the subject may be not affected by disorder or pathology characterized by an inflammatory response or go toward an amelioration of the disorder or pathology, respectively.

If by comparing the measured amount of the compound as above defined in a) (i.e. the C-terminus amino acid sequence of the human TSLP long isoform (SEQ ID NO: 2), or the corresponding sequence encoded from a TSLP orthologous or homologous gene, functional mutants, recombinant derivatives, fragments or analogues thereof) with the value obtained from a control sample, the amount of said compound in the test sample isolated from the subject corresponds to a similar or higher value, the subject may be not affected by disorder or pathology characterized by an inflammatory response or go toward an amelioration of the disorder or pathology, respectively.

If by comparing the measured amount of the compound as above defined in a) with the value obtained from a control sample, the amount of said compound in the test sample isolated from the subject corresponds to a lower value, the subject may present the disorder or pathology characterized by an inflammatory response or go towards an aggravation of the said disorder or pathology.

Alternatively, the expression “measuring the amount” is intended as measuring the alteration of the molecule. Said alteration can reflect an increase or a decrease in the amount of the compounds as above defined. An increase of b) can be correlated to an aggravation of the disease. A decrease of b) can be correlated to an amelioration of the disease or to recovery of the subject. A decrease of a) can be correlated to an aggravation of the disease. An increase of a) can be correlated to an amelioration of the disease or to recovery of the subject.

In a preferred embodiment, the value obtained from a control sample may be calculated as the ratio between the amount of the ratio between the amount of a) and b) according to the following formula: amount of a)/amount of b).

The method of the invention may be used to discriminate among different pathologies wherein, for example, the amount of one of the compound as above defined vary in the same way (e.g. it increases or decreases) in each pathology, while the amount of the other compound as above defined in one pathology vary (it increases or decreases), while in another pathology remain constant. For example, applying the method of the invention it is possible to discriminate between psoriasis and atopic dermatitis. Indeed, in the psoriasis the amount of the lTSLP increases, while the amount of the sTSLP doesn't change. Therefore, in this case an alteration of the ratio between the amount of the ratio between the amount of a) and b) according to the following formula: amount of a)/amount of b), compared to the value of a control sample, will be measured.

In the atopic dermatitis, the amount of the lTSLP increases, while the amount of the sTSLP will be reduced. Therefore, in this case a different alteration of the ratio between the amount of the ratio between the amount of a) and b) according to the following formula: amount of a)/amount of b), compared to the value of a control sample, will be measured.

A further object of the invention is a method for treating and/or preventing a disorder or pathology characterized by an inflammatory response comprising administering in a subject in need thereof an effective amount of a compound selected in the group consisting of:

a) a polypeptide consisting of the C-terminus amino acid sequence of the human Thymic stromal lymphopoietin (TSLP) long isoform (SEQ ID NO: 2), or a polypeptide consisting of the corresponding sequence encoded from a TSLP orthologous or homologous gene, functional mutants, recombinant or synthetic derivatives, fragments or analogues thereof;
b) a polynucleotide coding for said polypeptide;
c) a vector comprising said polynucleotide;
d) a host cell genetically engineered expressing said polypeptide.

In the present invention, the fragment of the C-terminus amino acid sequence of the human TSLP long isoform (SEQ ID NO: 2) is preferably the polypeptide as above defined.

Another object of the invention is a method for treating and/or preventing a disorder or pathology characterized by an inflammatory response comprising administering in a subject in need thereof an effective amount of a TSLP long isoform (SEQ ID NO:2) antagonist wherein said antagonist is:
an antibody or antibody fragment capable of binding selectively to the TSLP long isoform (SEQ ID NO:2) or its fragments and of neutralizing the activity of the TSLP long isoform:
an antibody or antibody fragment capable of binding selectively to the TSLP long isoform (SEQ ID NO:2) receptor; or
an isolated TSLP long isoform receptor.

Preferably the disorder or pathology characterized by an inflammatory response is Th1 and/or Th2-related.

Preferably the disorder or pathology characterized by an inflammatory response is selected from the group consisting of:

inflammatory bowel disease, colorectal cancer, psoriasis, atopic dermatitis, sepsis, sarcoidosis, condylomata accuminata, lichen ruber, basal cell carcinoma, actinic keratosis, lupus erythematodes, brain inflammation, allergy, allograft rejection and carcinoma.

Preferably the disorder or pathology characterized by an inflammatory response is celiac disease.

The compound as above defined are preferably biomarkers of the above disorders or pathologies.

Another object of the invention is an antibody or antigen-binding fragment thereof having binding specificity for human TSLP long isoform (SEQ ID NO: 1), wherein said antibody or antigen-binding fragment specifically binds human TSLP long isoform but does not bind human TSLP short isoform (SEQ ID NO:2) and its medical use, in particular in the treatment and/or prevention of a disorder or pathology characterized by an inflammatory response.

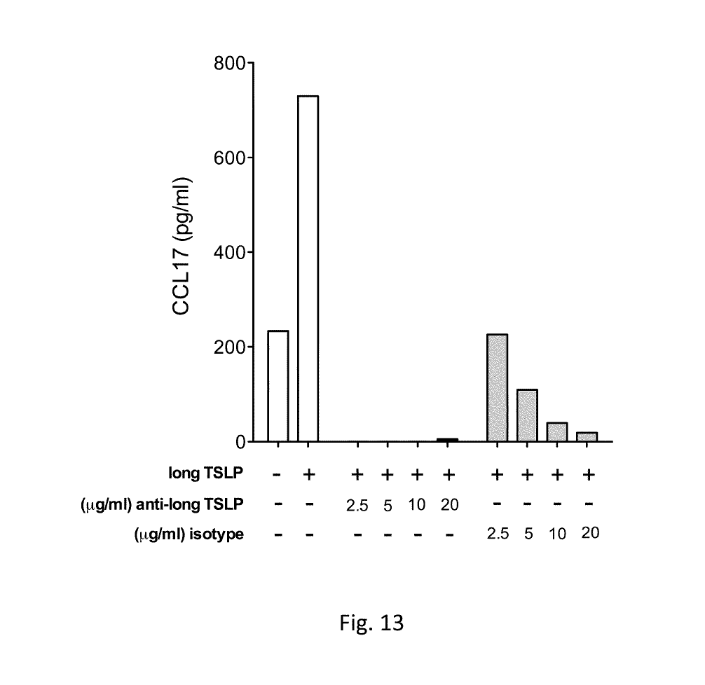

The antibody of the invention specifically targets a region comprised in the N-terminus of the lTSLP (i.e. aa. 1-96 of TSLP of SEQ ID NO:2). Since said region is not present in the sTSLP, the antibody of the invention doesn't recognize the sTLSP. The antibody of the invention has been shown to have a blocking activity (see FIG. 13).

Commercially available antibodies target the long TSLP but the specific target region of the long TSLP is not known. The provided antibody allows to discriminate between long and short TSLP, by targeting selectively the long isoform.

In the present invention a disorder or a pathology is characterized by an inflammatory response when in the tissue or in the blood there is an increase of inflammatory mediators, including TNF-a, IL-6, IL-8, IL-1b. Likewise, the tissue is characterized by an increase of infiltrating leukocytes.

In the present invention a Th1-related disorder or pathology is characterized by an increase in the production of IFN-g by infiltrating immune cells and/or by the recruitment of T cells expressing T-bet.

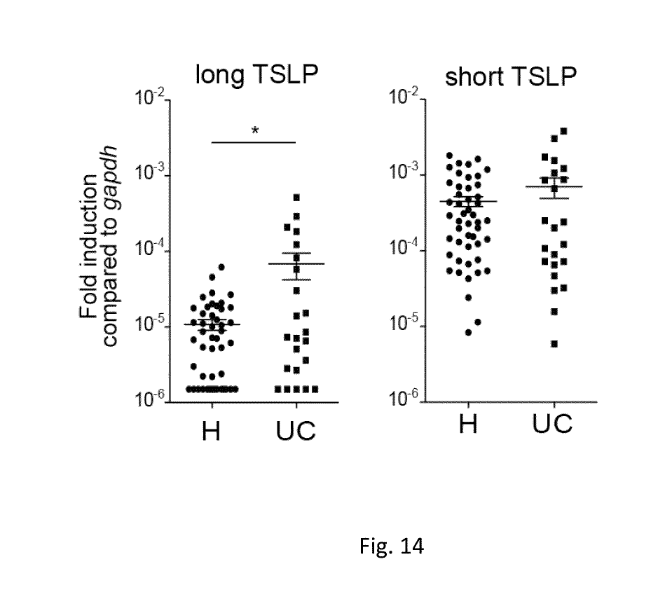

In the present invention, a preferred inflammatory bowel disease is ulcerative colitis. In ulcerative colitis the amount of short isoform of TSLP doesn't change, but there is an alteration of the ratio between the amount of lTSLP and shTSLP (see FIG. 14) compared to a normal subject.

The “subject in need thereof” or “the subject” may be a human or an animal, preferably Canis lupus familiaris, Felis catus, Equus caballus, Bos Taurus.

In the present invention “functional mutants” of the polypeptides are polypeptides that may be generated by mutating one or more amino acids in their sequences and that maintain their activity e.g. immunomodulatory activity or anti-inflammatory activity. Indeed, the polypeptide of the invention, if required, can be modified in vitro and/or in vivo, for example by glycosylation, myristoylation, amidation, carboxylation or phosphorylation, and may be obtained, for example, by synthetic or recombinant techniques known in the art.

In the present invention “functional” is intended for example as “maintaining their activity” e.g. immunomodulatory activity or anti-inflammatory activity.

The term “analogue” as used herein referring to a polypeptide means a modified peptide wherein one or more amino acid residues of the peptide have been substituted by other amino acid residues and/or wherein one or more amino acid residues have been deleted from the peptide and/or wherein one or more amino acid residues have been deleted from the peptide and or wherein one or more amino acid residues have been added to the peptide. Such addition or deletion of amino acid residues can take place at the N-terminal of the peptide and/or at the C-terminal of the peptide.

The term “derivative” as used herein in relation to a polypeptide means a chemically modified peptide or an analogue thereof, wherein at least one substituent is not present in the unmodified peptide or an analogue thereof, i.e. a peptide which has been covalently modified.

Typical modifications are amides, carbohydrates, alkyl groups, acyl groups, esters and the like. As used herein, the term “derivatives” also refers to longer or shorter polypeptides having e.g. a percentage of identity of at least 41%, preferably at least 41.5%, 50%, 54.9%, 60%, 61.2%, 64.1%, 65%, 70% or 75%, more preferably of at least 85%, as an example of at least 90%, and even more preferably of at least 95% with SEQ ID NO: 1, or with an amino acid sequence of the correspondent region encoded from a TSLP orthologous or homologous gene.

It has to be noted that the short isoform of TSLP in Homo sapiens is identical to the last 63 aa of the C-terminus portion of long TSLP. TSLP is highly conserved among species, for instance, in the mus musculus, dog and cat and cow it shares 41.5%, 61.2% and 64.1% and 54.9% identities with human TSLP, respectively.

As used herein “fragments” refers to polypeptides having preferably a length of at least 10 amino acids, more preferably at least 15, at least 17 amino acids or at least 20 amino acids, even more preferably at least 25 amino acids or at least 37 or 40 amino acids, and more preferably of at least 50 amino acids.

In a preferred embodiment, the fragment of long TSLP, also herein referred as “human TSLP long isoform (SEQ ID NO: 2)”, corresponds to the sequence aa. 29-159 of SEQ ID NO: 2.

As used herein, the expression “long TSLP” or “lTSLP” or “TSLP long isoform” may encompass the human Thymic stromal lymphopoietin (TSLP) long isoform (SEQ ID NO: 2) or a polypeptide consisting of the corresponding sequence encoded from a TSLP orthologous or homologous gene, functional mutants, recombinant or synthetic derivatives, fragments (as e.g. aa. 29-159 of SEQ ID NO: 2) or analogues thereof.

As used herein, the expression “short TSLP”, “shTSLP”, “sTSLP” or “TSLP short isoform” may encompass a polypeptide consisting of the C-terminus amino acid sequence of the human Thymic stromal lymphopoietin (TSLP) long isoform (SEQ ID NO: 2), or a polypeptide consisting of the corresponding sequence encoded from a TSLP orthologous or homologous gene, functional mutants, recombinant or synthetic derivatives, fragments or analogues thereof. Preferably, it encompasses the polypeptide of SEQ ID NO:1, or fragments thereof as aa. 4-63, 4-40 or 24-63 of the sequence of the TSLP short isoform (SEQ ID NO: 1).

As used herein, “percentage of identity” between two amino acids sequences, means the percentage of identical amino-acids, between the two sequences to be compared, obtained with the best alignment of said sequences, this percentage being purely statistical and the differences between these two sequences being randomly spread over the amino acids sequences. As used herein, “best alignment” or “optimal alignment”, means the alignment for which the determined percentage of identity (see below) is the highest. Sequences comparison between two amino acids sequences are usually realized by comparing these sequences that have been previously aligned according to the best alignment; this comparison is realized on segments of comparison in order to identify and compared the local regions of similarity. The best sequences alignment to perform comparison can be realized, beside by a manual way, by using the global homology algorithm developed by SMITH and WATERMAN (Ad. App. Math., vol. 2, p: 482, 1981), by using the local homology algorithm developed by NEDDLEMAN and WUNSCH (J. MoI. Biol, vol. 48, p: 443, 1970), by using the method of similarities developed by PEARSON and LIPMAN (Proc. Natl. Acd. Sci. USA, vol. 85, p: 2444, 1988), by using computer softwares using such algorithms (GAP, BESTFIT, BLAST P, BLAST N, FASTA, TFASTA in the Wisconsin Genetics software Package, Genetics Computer Group, 575 Science Dr., Madison, Wis. USA), by using the MUSCLE multiple alignment algorithms (Edgar, Robert C, Nucleic Acids Research, vol. 32, p: 1792, 2004). To get the best local alignment, one can preferably used BLAST software, with the BLOSUM 62 matrix, or the PAM 30 matrix. The identity percentage between two sequences of amino acids is determined by comparing these two sequences optimally aligned, the amino acid sequences being able to comprise additions or deletions in respect to the reference sequence in order to get the optimal alignment between these two sequences. The percentage of identity is calculated by determining the number of identical position between these two sequences, and dividing this number by the total number of compared positions, and by multiplying the result obtained by 100 to get the percentage of identity between these two sequences.

The term “polynucleotide” according to the present invention refers to a single strand nucleotide chain or its complementary strand which can be of the DNA or RNA type, or a double strand nucleotide chain which can be of the cDNA (complementary) or genomic DNA type. Preferably, the polynucleotides of the invention are of the DNA type, namely double strand DNA. The term “polynucleotide” also refers to modified polynucleotides.

The polynucleotides of this invention are isolated or purified from their natural environment. Preferably, the polynucleotides of this invention can be prepared using conventional molecular biology techniques such as those described by Sambrook et al. (Molecular Cloning: A Laboratory Manual, 1989) or by chemical synthesis.

The polynucleotide of the invention may also include the coding sequence of the polypeptide defined previously, additional coding sequence such as leader sequence or a proprotein sequence, and/or additional non-coding sequence, such as introns or 5′ and/or 3′ UTR sequences.

As used herein, the term “vector” refers to an expression vector, and may be for example in the form of a plasmid, a viral particle, a phage, etc. Such vectors may include bacterial plasmids, phage DNA, baculovirus, yeast plasmids, vectors derived from combinations of plasmids and phage DNA, viral DNA such as vaccinia, adenovirus, fowl pox virus, and pseudorabies. Large numbers of suitable vectors are known to those of skill in the art and are commercially available. The following vectors are provided by way of example. Bacterial: pQE70, pQE60, pQE-9 (QIAGEN), pbs, pDIO, phagescript, psiX174, pbluescript SK, pbsks, pNH8A, pNH1[beta]a, pNH18A, pNH46A (STRATAGENE), ptrc99a, pKK223-3, pKK233-3, pDR540, pRIT5 (PHARMACIA). Eukaryotic: pWLNEO, pSV2CAT, pOG44, pXT1, pSG (STRATAGENE), pSVK3, pBPV, pMSG, pSVL (PHARMACIA). However, any other vector may be used as long as it is replicable and viable in the host. The polynucleotide sequence, preferably the DNA sequence in the vector is operatively linked to an appropriate expression control sequence(s) (promoter) to direct mRNA synthesis. As representative examples of such promoters, one can mention prokaryotic or eukaryotic promoters such as CMV immediate early, HSV thymidine kinase, early and late SV40, LTRs from retrovirus, and mouse metallothionein-I. The expression vector also contains a ribosome binding site for translation initiation and a transcription vector. The vector may also include appropriate sequences for amplifying expression. In addition, the vectors preferably contain one or more selectable marker genes to provide a phenotypic trait for selection of transformed host cells such as dihydro folate reductase or neomycin resistance for eukaryotic cell culture, or such as tetracycline or ampicillin resistance in E. coli.

As used herein, the term “host cell genetically engineered” relates to host cells which have been transduced, transformed or transfected with the polynucleotide or with the vector described previously. As representative examples of appropriate host cells, one can cite bacterial cells, such as E. coli, Streptomyces, Salmonella typhimurium, fungal cells such as yeast, insect cells such as Sf9, animal cells such as CHO or COS, plant cells, etc. The selection of an appropriate host is deemed to be within the scope of those skilled in the art from the teachings herein. Preferably, said host cell is an animal cell, and most preferably a human cell. The introduction of the polynucleotide or of the vector described previously into the host cell can be effected by method well known from one of skill in the art such as calcium phosphate transfection, DEAE-Dextran mediated transfection, or electroporation. The polynucleotide may be a vector such as for example a viral vector. Another object of the invention is a composition comprising a transformed host cell expressing a peptide selected from the peptide of SEQ ID NO: 1.

The man skilled in the art is well aware of the standard methods for incorporation of a polynucleotide into a host cell, for example transfection, lipofection, electroporation, microinjection, viral infection, thermal shock, transformation after chemical permeabilisation of the membrane or cell fusion.

The term “antibody” is used herein in the broadest sense and specifically covers monoclonal antibodies of any isotype such as IgG, IgM, IgA, IgD and IgE, polyclonal antibodies, chimeric antibodies, humanized antibodies and antibody fragments. An antibody reactive with a specific antigen can be generated by recombinant methods such as selection of libraries of recombinant antibodies in phage or similar vectors, or by immunizing an animal with the antigen or an antigen-encoding nucleic acid. A typical IgG antibody is comprised of two identical heavy chains and two identical light chains that are joined by disulfide bonds. Each heavy and light chain contains a constant region and a variable region. Each variable region contains three segments called “complementarity-determining regions” (“CDRs”) or “hypervariable regions”, which are primarily responsible for binding an epitope of an antigen. They are usually referred to as CDR1, CDR2, and CDR3, numbered sequentially from the N-terminus. The more highly conserved portions of the variable regions are called the “framework regions”. As used herein, “VH” refers to the variable region of an immunoglobulin heavy chain of an antibody, including the heavy chain of an Fv, scFv, dsFv, Fab, Fab′ or F(ab′)2 fragment. Reference to “VL” refers to the variable region of the immunoglobulin light chain of an antibody, including the light chain of an Fv, scFv, dsFv, Fab, Fab′ or F(ab′)2 fragment. A “polyclonal antibody” is an antibody which was produced among or in the presence of one or more other, non-identical antibodies. In general, polyclonal antibodies are produced from a B-lymphocyte in the presence of several other B-lymphocytes producing non-identical antibodies. Usually, polyclonal antibodies are obtained directly from an immunized animal. A “monoclonal antibody”, as used herein, is an antibody obtained from a population of substantially homogeneous antibodies, i.e. the antibodies forming this population are essentially identical except for possible naturally occurring mutations which might be present in minor amounts. These antibodies are directed against a single epitope and are therefore highly specific. An “epitope” is the site on the antigen to which an antibody binds. As used herein, a “chimeric antibody” is an antibody in which the constant region, or a portion thereof, is altered, replaced, or exchanged, so that the variable region is linked to a constant region of a different species, or belonging to another antibody class or subclass. “Chimeric antibody” also refers to an antibody in which the variable region, or a portion thereof, is altered, replaced, or exchanged, so that the constant region is linked to a variable region of a different species, or belonging to another antibody class or subclass. Methods for producing chimeric antibodies are known in the art.

The term “humanized antibody”, as used herein, refers to a chimeric antibody which contains minimal sequence derived from non-human immunoglobulin. The goal of humanization is a reduction in the immunogenicity of a xenogenic antibody, such as a murine antibody, for introduction into a human, while maintaining the full antigen binding affinity and specificity of the antibody. Humanized antibodies, or antibodies adapted for non-rejection by other mammals, may be produced using several technologies such as resurfacing and CDR grafting. Humanized chimeric antibodies preferably have constant regions and variable regions other than the complementarity determining regions derived substantially or exclusively from the corresponding human antibody regions and CDRs derived substantially or exclusively from a mammal other than a human. The antibodies of the present invention include both the full length antibodies discussed above, as well as epitope-binding fragments thereof. As used herein, “antibody fragments” include any portion of an antibody that retains the ability to bind to the epitope recognized by the full length antibody, generally termed “epitope-binding fragments.” Examples of antibody fragments include, but are not limited to, Fab, Fab′ and F(ab′)2, Fd, single-chain Fvs (scFv), single-chain antibodies, disulfide-linked Fvs (dsFv) and fragments comprising either a VL or VH region. Epitope-binding fragments, including single-chain antibodies, may comprise the variable region(s) alone or in combination with the entirety or a portion of the following: hinge region, CH1, CH2, and CH3 domains.

The terms “treat or treatment” and “prevent or prevention” as well as words stemming therefrom, as used herein, do not necessarily imply 100% or complete treatment or prevention. Rather, there are varying degrees of treatment or prevention of which one of ordinary skill in the art recognizes as having a potential benefit or therapeutic effect, in this respect, the inventive methods can provide any amount of any level of treatment or prevention of a condition associated with inflammation, e.g. in a mammal. Furthermore, the treatment or prevention provided by the inventive method can include treatment or prevention of one or more conditions or symptoms of the disease being treated or prevented. Also, for purposes herein, “prevention” can encompass delaying the onset of the disease, or a symptom or condition thereof. According to the present invention, an “effective amount” of a composition is one which is sufficient to achieve a desired biological effect, in this case a decrease in inflammatory response in the animal or human. It is understood that the effective dosage will be dependent upon the age, sex, health, and weight of the recipient, kind of concurrent treatment, if any, frequency of treatment, and the nature of the effect desired. The preferred dosage can be tailored to the individual subject, as is understood and determinable by one of skill in the art, without undue experimentation. Examples of ranges of effective doses of the above antagonist or compound of the invention (from 1 mg/kg to 100 mg/kg, in particular systemically, topically, locally (e.g. rectally) and orally administered) are not intended to limit the invention and represent preferred dose ranges.

The present invention has use in human and animal health (veterinary use), preferably in Canis lupus familiaris, Felis catus, Equus caballus, Bos Taurus.

Amounts effective for a therapeutic or prophylactic use will depend on, for example, the stage and severity of the disease or disorder being treated and the judgment of the prescribing physician. The size of the dose will also be determined by the compound selected, method of administration, timing and frequency of administration as well as the existence, nature, and extent of any adverse side-effects that might accompany the administration of a particular compound and the desired physiological effect. It will be appreciated by one of skill in the art that various diseases or disorders could require prolonged treatment involving multiple administrations, perhaps using the compound of the invention in each or various rounds of administration. The disclosed compounds can be administered in a composition (e.g., pharmaceutical composition) that can comprise at least one excipient (e.g., a pharmaceutically acceptable excipient), as well as other therapeutic agents (e.g., anti-inflammatory agents). The composition can be administered by any suitable route, including parenteral, topical, oral, or local administration. The pharmaceutically acceptable excipient is preferably one that is chemically inert to the compounds above disclosed and one that has little or no side effects or toxicity under the conditions of use. Such pharmaceutically acceptable carriers include, but are not limited to, water, saline, Cremophor EL (Sigma Chemical Co., St. Louis, Mo.), propylene glycol, polyethylene glycol, alcohol, and combinations thereof. The choice of carrier will be determined in part by the particular compound as well as by the particular method used to administer the composition. Accordingly, there is a wide variety of suitable formulations of the composition. The pharmaceutical composition in the context of an embodiment of the invention can be, for example, in the form of a pill, capsule, or tablet, each containing a predetermined amount of one or more of the active compounds and preferably coated for ease of swallowing, in the form of a powder or granules, or in the form of a solution or suspension. For oral administration, fine powders or granules may contain diluting, dispersing, and or surface active agents and may be present, for example, in water or in a syrup, in capsules or sachets in the dry state, or in a nonaqueous solution or suspension wherein suspending agents may be included, or in tablets wherein binders and lubricants may be included. Components such as sweeteners, flavoring agents, preservatives (e.g., antimicrobial preservatives), suspending agents, thickening agents, and/or emulsifying agents also may be present in the pharmaceutical composition. When administered in the form of a liquid solution or suspension, the formulation can contain one or more of the active compounds and purified water. Optional components in the liquid solution or suspension include suitable preservatives (e.g., antimicrobial preservatives), buffering agents, solvents, and mixtures thereof. A component of the formulation may serve more than one function. Preservatives may be used. Suitable preservatives may include, for example, methylparaben, propylparaben, sodium benzoate, and benzalkonium chloride. A mixture of two or more preservatives optionally may be used. The preservative or mixtures thereof are typically present in an amount of about 0.0001% to about 2% by weight of the total composition. Suitable buffering agents may include, for example, citric acid, sodium citrate, phosphoric acid, potassium phosphate, and various other acids and salts. A mixture of two or more buffering agents optionally may be used. The buffering agent or mixtures thereof are typically present in an amount of about 0.001% to about 4% by weight of the total composition. The following formulations for oral, aerosol, parenteral (e.g., subcutaneous, intravenous, intraarterial, intramuscular, intradermal, interperitoneal, and intrathecal), and rectal administration are merely exemplary and are in no way limiting.

Formulations suitable for oral administration can consist of (a) liquid solutions, such as an effective amount of the compound dissolved in diluents, such as water, saline, or orange juice; (b) capsules, sachets, tablets, lozenges, and troches, each containing a predetermined amount of the active ingredient, as solids or granules; (c) powders; (d) suspensions in an appropriate liquid; and (e) suitable emulsions. Liquid formulations may include diluents, such as water and alcohols, for example, ethanol, benzyl alcohol, and the polyethylene alcohols, either with or without the addition of a pharmaceutically acceptable surfactant, suspending agent, or emulsifying agent. Capsule forms can be of the ordinary hard- or soft-shelled gelatin type containing, for example, surfactants, lubricants, and inert fillers, such as lactose, sucrose, calcium phosphate, and cornstarch. Tablet forms can include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and pharmacologically compatible carriers. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such carriers as are known in the art. The above compounds, alone or in combination with other suitable components, can be made into aerosol formulations to be administered via inhalation. These aerosol formulations can be placed into pressurized acceptable propellants, such as dichlorodifluoromethane, propane, nitrogen, and the like. They also may be formulated as pharmaceuticals for non-pressured preparations, such as in a nebulizer or an atomizer. Formulations suitable for parenteral administration include aqueous and nonaqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The above compounds may be administered in a physiologically acceptable diluent in a pharmaceutical carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol, glycerol ketals, such as 2,2-dimethyl-1,3-dioxolane-4-methanol, ethers, such as poly(ethyleneglycol) 400, an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants. Oils, which can be used in parenteral formulations, include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters.

Suitable soaps for use in parenteral formulations may include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylene-polypropylene copolymers, (d) amphoteric detergents such as, for example, alkyl-beta-aminopropionates, and 2-alkyl-imidazoline quaternary ammonium salts, and (3) mixtures thereof.

Suitable preservatives and buffers can be used in such formulations. In order to minimize or eliminate irritation at the site of injection, such compositions may contain one or more nonionic surfactants having a hydrophile-lipophile balance (HLB) of from about 12 to about 17. The quantity of surfactant in such formulations ranges from about 5% to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampoules and vials, and can be stored in a freeze-dried (lyophilized) condition requiring only the addition of the sterile liquid carrier, for example, water, for injections, immediately prior to use. Extemporaneous injection solutions and suspensions can be prepared from sterile powders, granules, and tablets. The above compounds may be administered as an injectable formulation. The requirements for effective pharmaceutical carriers for injectable compositions are well known to those of ordinary skill in the art. See Pharmaceutics and Pharmacy Practice, J. B. Lippincott Co., Philadelphia, Pa., Banker and Chalmers, eds., pages 238-250 (1982), and ASHP Handbook on Injectable Drugs, Toissel, 4th ed., pages 622-630 (1986). Topical formulations, including those that are useful for transdermal drug release, are well known to those of skill in the art and are suitable in the context of embodiments of the invention for application to skin. The concentration of a compound of embodiments of the invention in the pharmaceutical formulations can vary, e.g., from less than about 1%, usually at or at least about 10%, to as much as 20% to 50% or more by weight, and can be selected primarily by fluid volumes, and viscosities, in accordance with the particular mode of administration selected. Methods for preparing administrable (e.g., parenterally administrable) compositions are known or apparent to those skilled in the art and are described in more detail in, for example, Remington's Pharmaceutical Science (17th ed., Mack Publishing Company, Easton, Pa., 1985). In addition to the aforedescribed pharmaceutical compositions, the above compounds can be formulated as inclusion complexes, such as cyclodextrin inclusion complexes, or liposomes. Liposomes can serve to target the compounds to a particular tissue. Many methods are available for preparing liposomes, as described in, for example, Szoka et al., Ann. Rev. Biophys. Bioeng., 9:467 (1980) and U.S. Pat. Nos. 4,235,871, 4,501,728, 4,837,028, and 5,019,369.

Preferably, the compound as above described may be formulated for oral or local administration in a sustained or controlled release acid resistant delivery system.

When the agent of the invention is administered with one or more additional therapeutic agents, one or more additional therapeutic agents can be coadministered to the mammal. By “coadministering” is meant administering one or more additional therapeutic agents and the above compound sufficiently close in time such that the compound can enhance the effect of one or more additional therapeutic agents. In this regard, the compound can be administered first and the one or more additional therapeutic agents can be administered second, or vice versa. Alternatively, the compound and the one or more additional therapeutic agents can be administered simultaneously. The delivery systems useful in the context of embodiments of the invention may include time-released, delayed release, and sustained release delivery systems such that the delivery of the inventive composition occurs prior to, and with sufficient time to cause, sensitization of the site to be treated. The inventive composition can be used in conjunction with other therapeutic agents or therapies. Such systems can avoid repeated administrations of the inventive composition, thereby increasing convenience to the subject and the physician, and may be particularly suitable for certain composition embodiments of the invention. Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are lipids including sterols such as cholesterol, cholesterol esters, and fatty acids or neutral fats such as mono-di- and tri-glycerides; hydrogel release systems; sylastic systems; peptide based systems; wax coatings; compressed tablets using conventional binders and excipients; partially fused implants; and the like. Specific examples include, but are not limited to: (a) erosional systems in which the active composition is contained in a form within a matrix such as those described in U.S. Pat. Nos. 4,452,775, 4,667,014, 4,748,034, and 5,239,660 and (b) diffusional systems in which an active component permeates at a controlled rate from a polymer such as described in U.S. Pat. Nos. 3,832,253 and 3,854,480. In addition, pump-based hardware delivery systems can be used, some of which are adapted for implantation.

In a preferred aspect of the invention, the compound according to the invention can inhibit the cytokine IFNgamma by 50% at a concentration of 25 ng/ml.

In a preferred embodiment of the method for the screening of a therapeutic treatment or for monitoring the efficacy of a therapeutic treatment of a disorder or pathology characterized by an inflammatory response, the disorder is celiac disease and the therapy is a gluten free diet.

In a preferred embodiment of the compound as above defined, the polypeptide comprises a sequence consisting essentially of the aa. 24-40 of the amino acid sequence of the TSLP short isoform (SEQ ID NO: 1), or of the amino acid sequence of the corresponding region encoded from a TSLP orthologous or homologous gene, functional mutants, recombinant or synthetic derivatives, fragments or analogues thereof.

In the present invention, immunomodulatory activity refers to the ability of modifying or regulating one or more immune functions (Farlex Partner Medical Dictionary © Farlex 2012), in example the anti-inflammatory activity.

In the present invention, anti-inflammatory or antiinflammatory refers to the property of a substance or treatment that reduces inflammation. In the present invention, preferred thymic stromal lyphopoietin (TSLP) encoded by orthologous or homologous genes are:

gi|354484147|ref|XP_003504252.1| PREDICTED: thymic stromal lymphopoietin-
like [Cricetulus griseus] (SEQ ID NO: 9) Nucleotide: XM_003504204.1 (SEQ ID NO: 25)
gi|255759970|ref|NP_001157535.1| thymic stromal lymphopoietin precursor
[Equus caballus] (SEQ ID NO: 10) Nucleotide: NM_001164063.2 (SEQ ID NO: 26)
gi|403256664|ref|XP_003920983.1| PREDICTED: thymic stromal lymphopoietin
[Saimiri boliviensis boliviensis] (SEQ ID NO: 11) Nucleotide: XM_003920934.1 (SEQ ID
NO: 27)
gi|296193991|ref|XP_002744731.1| PREDICTED: thymic stromal lymphopoietin
[Callithrix jacchus] (SEQ ID NO: 12) Nucleotide: XM_002744685.1 (SEQ ID NO: 28)
gi|109078157|ref|XP_001100503.1| PREDICTED: thymic stromal lymphopoietin
[Macaca mulatta] (SEQ ID NO: 13) Nucleotide: XM_001100503.2 (SEQ ID NO: 29)
gi|402872238|ref|XP_003900034.1| PREDICTED: thymic stromal lymphopoietin
[Papio anubis] (SEQ ID NO: 14) Nucleotide: XM_003899985.1 (SEQ ID NO: 30)
gi|332221439|ref|XP_003259868.1| PREDICTED: thymic stromal lymphopoietin
[Nomascus leucogenys] (SEQ ID NO: 15) Nucleotide: XM_003259820.2 (SEQ ID NO: 31)
gi|297675754|ref|XP_002815824.1| PREDICTED: thymic stromal lymphopoietin
[Pongo abelii] (SEQ ID NO: 16) Nucleotide: XM_002815778.2 (SEQ ID NO: 32)
gi|410949156|ref|XP_003981290.1| PREDICTED: thymic stromal lymphopoietin
[Felis catus] (SEQ ID NO: 17) Nucleotide: XM_003981241.2 (SEQ ID NO: 33)
gi|114601106|ref|XP_001141816.1| PREDICTED: thymic stromal lymphopoietin
isoform 2 [Pan troglodytes] (SEQ ID NO: 18) Nucleotide: XM_001141816.3 (SEQ ID NO:
34)
gi|397512961|ref|XP_003826800.1| PREDICTED: thymic stromal lymphopoietin
[Pan paniscus] (SEQ ID NO: 19) Nucleotide: XM_003826752.1 (SEQ ID NO: 35)
gi|426349610|ref|XP_004042385.1| PREDICTED: thymic stromal lymphopoietin
isoform 1[Gorilla gorilla gorilla] (SEQ ID NO: 20) Nucleotide: XM_004042337.1 (SEQ ID
NO: 36)
gi|385214965|gb|AFI49343.1| thymic stromal lymphopoietin, partial
[Canis lupus familiaris] (SEQ ID NO: 21) Nucleotide: JQ698664.1 (SEQ ID NO: 37)
gi|301767608|ref|XP_002919224.1| PREDICTED: thymic stromal lymphopoietin-
like, partial [Ailuropoda melanoleuca] (SEQ ID NO: 22) Nucleotide: XM _002919178.1
(SEQ ID NO: 38)
gi|10946698|ref|NP_067342.1| thymic stromal lymphopoietin precursor [Mus
musculus] (SEQ ID NO: 23) Nucleotide: NM_021367.2 (SEQ ID NO: 39)
gi|296482930| tpg|DAA25045.1| thymic stromal lymphopoietin-like [Bos taurus]
(SEQ ID NO: 24) Nucleotide: GJ061991.1

In the present invention, “fragments of the corresponding sequence encoded from a TSLP orthologous or homologous gene” (or the fragments relative to short isoform) can be for example the following sequences derived from aligned sequences:

gi|354484147|ref|XP_003504252.1|
Seq ID: 9
IFALHTKATLISQCPGYSETQRNNAQEMKLEV----KDICLNQTSQIQFLWHSLLQTLKY-- (1)
gi|255759970|ref|NP_001157535.1|
Seq ID: 10
IV-TATNATLNSHCPGHSGIQINNTQAMKKRKKREVTTNKCLKQVSNLIELWRYFSRSQ---- (2)
gi|403256664|ref|XP_003920983.1|
Seq ID: 11
LFAVRTNATLALWCPGYSETQINDTQAMKKRKKRKVTTNKCLEQVSQLQGLWRRFIRTLFK-- (3)
gi|296193991|ref|XP_002744731.1|
Seq ID: 12
LFAVRTNATLALWCPGYSETQINATQEMKKRKKRKVTTNKCLEQVSQLSGLWRRFIRTLRK-- (4)
gi|109078157|ref|XP_001100503.1|
Seq ID: 13
MFARKTKATLALWCPGYSETQINATQAMKKRRKRKVTTNRCLEQVSQLLSLWRRFIRTLLKKQ (5)
gi|402872238|ref|XP_003900034.1|
Seq ID: 14
MFARKTKATLALWCPGYSETQINATQAMKKRRKRKVTTNKCLEQVSQLLGLWRRFIRTLLKKQ (6)
gi|332221439|ref|XP_003259868.1|
Seq ID: 15
MFAMKTKAALALWCPGYSETQINATQAMKKRRKRKVTTNKCLEQVSQLLGLWRRFSRRLLKQQ (7)
gi|297675754|ref|XP_002815824.1|
Seq ID: 16
VFAMKTKAALALWCPGYSETQINATQAMKKRRKRKVTTNKCLEQVSQLLGLWRRFNRRLLKQQ (8)
gi|410949156|ref|XP_003981290.1|
Seq ID: 17
IFAIRTNATLTLQCPGYSETQINNTQAKKKRKKRQVTTNKCREQVSHLMELWRRFSRIS---- (9)
gi|114601106|ref|XP_001141816.1|
Seq ID: 18
MFAMKTKAALTIWCPGYSETQINATQAMKKRRKRKVTTNKCLEQVSQLQGLWRRFNRPLLKQQ (10)
gi|397512961|ref|XP_003826800.1|
Seq ID: 19
MFAMKTKAALAIWCPGYSETQINATQAMKKRRKRKVTTNKCLEQVSQLQGLWRRFNRPLLKQQ (11)
gi|426349610|ref|XP_004042385.1|
Seq ID: 20
MFAMKTKAALAIWCPGYSETQINATQAMKKRRKRKVTTNKCLEQVSQLQGLWRRFNRPLLKQQ (12)
gi|385214965|gb|AFI49343.1|
Seq ID: 21
AFAEGTVAALAAECPGYSETQINNTQAKKKRKKRGVTTNKCREQVAHLIGLWRRFSRIS---- (13)
gi|301767608|ref|XP_002919224.1|
Seq ID: 22
IFAIGTRATLTLQCPGYSETQINNTQAKKKRKKRKVTTNKCREQVAYLIGLWRRFSRIS---- (14)
gi|10946698|ref|NP_067342.1|
Seq ID: 23
TFARRTREALNDHCPGYSETQRNDGTQEMAQE---VQNICLN-QTSQILRLWYSFMQSPE--- (15)
gi|296482930|tpg|DAA25045.1|
Seq ID: 24
AFAVRTHAALAAACPGYSETQVSRATQSARRVPRASERGAGPGEPRPPAARRRAWTRALTSEG (16)
gi|190886449|ref|NP_612561.2|
Seq ID: 1
MFAMKTKAALAIWCPGYSETQINATQAMKKRRKRKVTTNKCLEQVSQLQGLWRRFNRPLLKQQ (17)
(1)corresponding to aa.121-176 of Seq ID NO: 9
(2)corresponding to aa.86-143 of Seq ID NO: 10
(3)corresponding to aa.97-157 of Seq ID NO: 11
(4)corresponding to aa.97-157 of Seq ID NO: 12
(5)corresponding to aa.97-159 of Seq ID NO: 13
(6)corresponding to aa.97-159 of Seq ID NO: 14
(7)corresponding to aa.97-159 of Seq ID NO: 15
(8)corresponding to aa.97-159 of Seq ID NO: 16
(9)corresponding to aa.125-183 of Seq ID NO: 17
(10)corresponding to aa.97-159 of Seq ID NO: 18
(11)corresponding to aa.97-159 of Seq ID NO: 19
(12)corresponding to aa.43-105 of Seq ID NO: 20
(13)corresponding to aa.96-154 of Seq ID NO: 21
(14)corresponding to aa.90-148 of Seq ID NO: 22
(15)corresponding to aa.85-140 of Seq ID NO: 23
(16)corresponding to aa.88-150 of Seq ID NO: 24
(17)corresponding to SEQ ID NO: 1

The invention will be now described by the following non-limiting examples referring to the following figures:

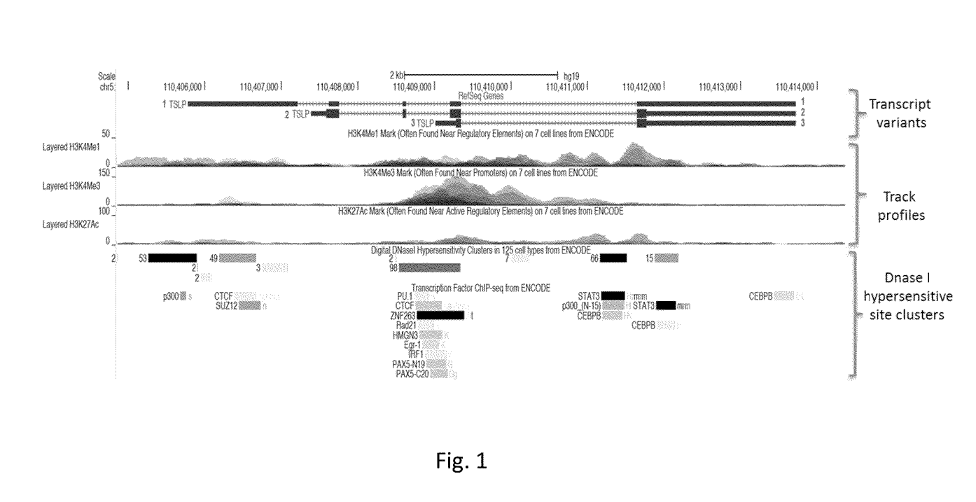

FIG. 1: Different existing isoforms of human TSLP. Schematic representation of human TSLP locus from UCSC Genome Browser (hg19). On the top: three transcript variants (RefSeq database (http://genome.ucsc.edu and in particular for the TSLP: http://genome-euro.ucsc.edu/cgi-bin/hgTracks?db=hg19&position=chr5%3A110404856-110414355&hgsid=197243096_TsWaq2VphlzNP2DIjsuWKs3iDOt) are portrayed, two of which give rise to coding RNA (2 and 3). On the middle: ENCODE track profiles of layered H3K4Me1, H3K4Me3 and H3K27Ac that mark regulatory elements/enhancer, promoters and active regulatory elements, respectively. On the bottom: ENCODE DNaseI hypersensitive site clusters derived from 125 cell types and transcription factor binding regions assessed by ChIP-seq experiments

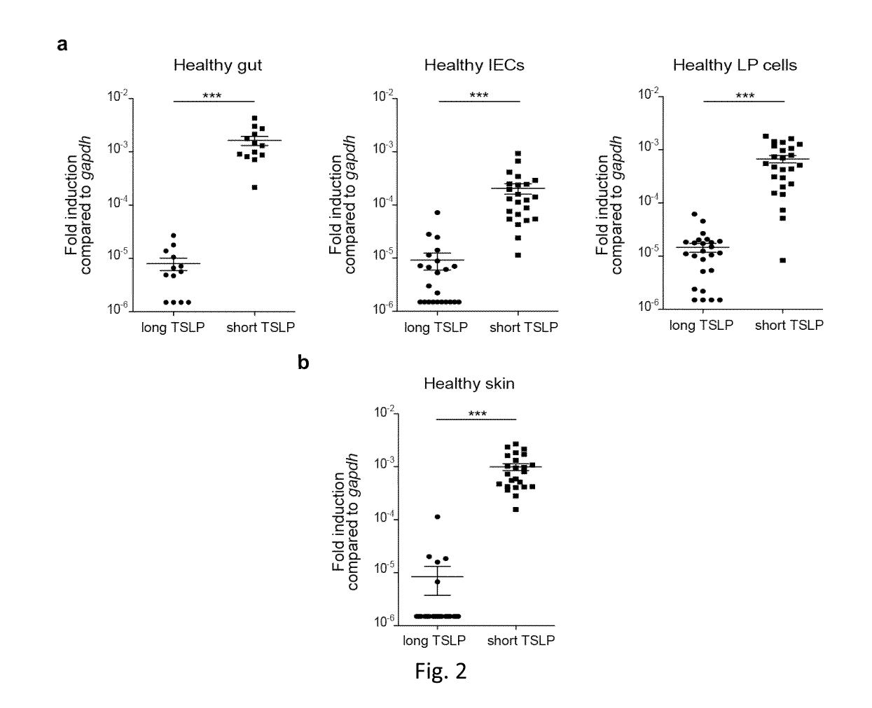

FIG. 2: Short TSLP is the predominant isoform expressed on human intestinal and skin tissue. Quantitative real-time PCR analysis of short and long TSLP mRNA in intestinal tissue samples (gut), isolated epithelial (IECs) and lamina propria (LP) cells (a) and skin tissue samples (b) from healthy individuals. The ‘fold induction’ in mRNA expression is in reference to that of the ‘housekeeping’ gene, Gapdh. ***p<0.001

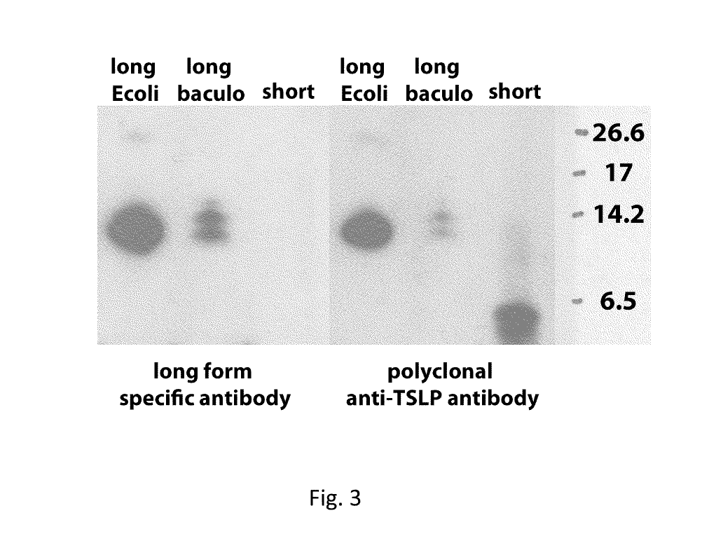

FIG. 3: Cloning, expression and purification of the two isoforms. Western blot of commercially available TSLP (long Ecoli, R&D systems) and in-house produced long and short isoforms carried out with two antibodies, one commercially available and recognizing both isoforms (right), and one in-house made recognizing only the long isoform (the short isoform disappears). Molecular weight are expressed in KDa.

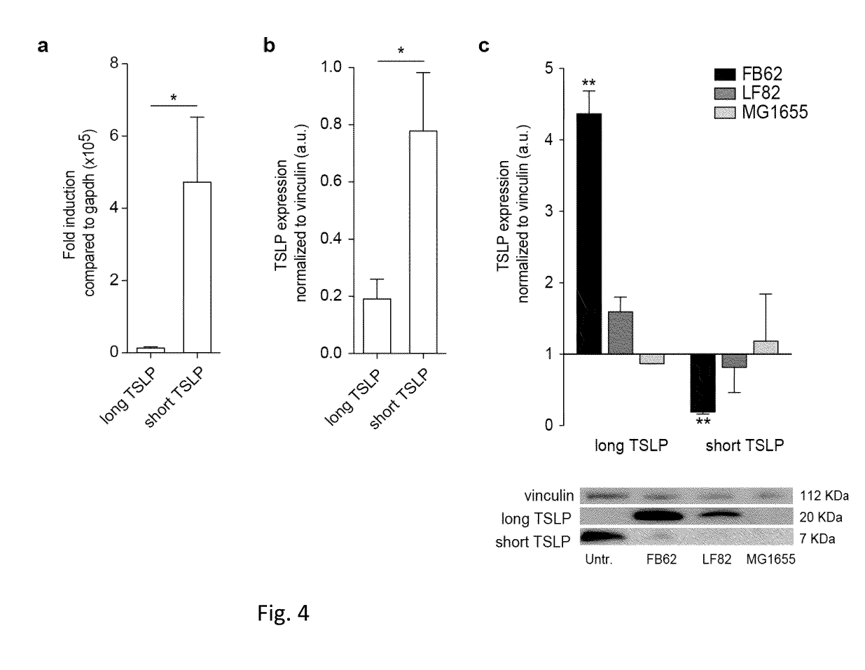

FIG. 4: TSLP isoforms are differentially regulated in a human epithelial cell line.

(a) Quantitative real-time PCR analysis of short and long TSLP mRNA in polarized, untreated Caco2 cells. The ‘fold induction’ in mRNA expression is in reference to that of the ‘housekeeping’ gene, Gapdh. * p<0.05. (b, c) Quantification of TSLP isoform protein expression in polarized Caco2 cells. Lysates from cells left untreated (b) or challenged with different bacterial strains (Salmonella enterica, serovar typhimurium SL1344: FB62; Escherichia coli strain LF82; Escherichia coli MG1655) (c) were immunoblotted with antibodies against vinculin, long TSLP isoform and total TSLP isoforms. Protein levels are normalized to vinculin expression. In (c) data are expressed as fold change compared with untreated cells (a.u.=1). Data are shown as Mean±SD of three independent experiments. * p<0.05, ** p<0.01.

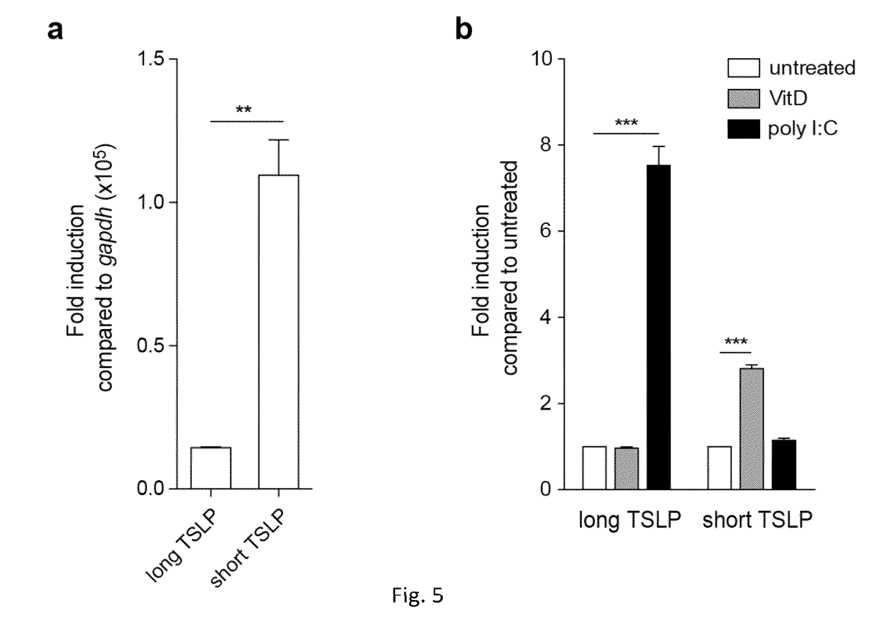

FIG. 5: TSLP isoforms are differentially regulated in human keratinocytes.

Quantitative real-time PCR analysis of short and long TSLP mRNA in untreated (a) or stimulated with vitamin D or poly(I:C) (b) HaCaT cells. The ‘fold induction’ in mRNA expression is in reference to that of the ‘housekeeping’ gene, Gapdh. In (b) data are expressed as fold change compared with untreated cells (a.u.=1). Data are shown as Mean±SD of three independent experiments. ** p<0.01, *** p<0.001.

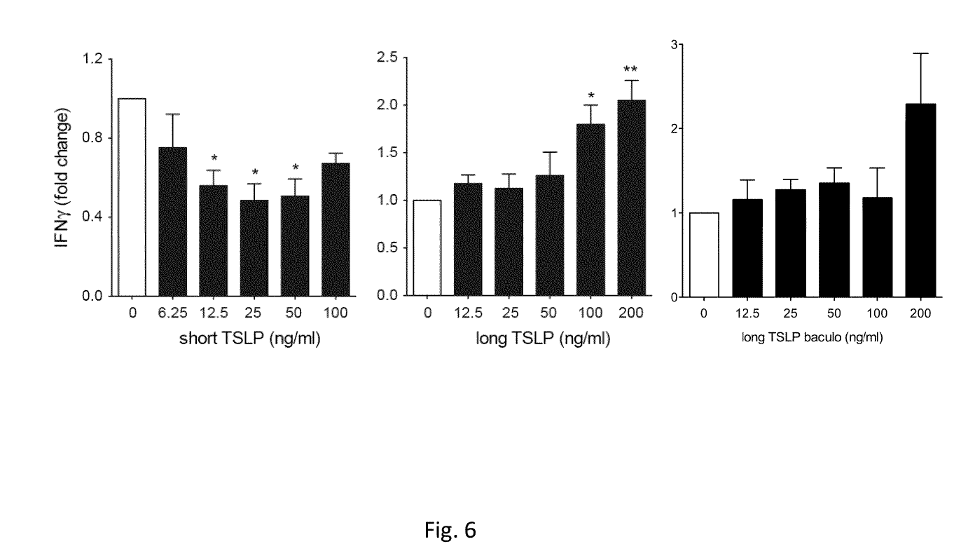

FIG. 6: Short TSLP has anti-inflammatory properties. Quantification of IFNgamma production in a bidirectional mixed lymphocyte reaction (MLR) performed with peripheral blood mononuclear cells (PBMC) from two different donors (ratio 1:1) in the presence of increasing concentration of short TSLP (left) and long TSLP produced in E. coli (middle) or in a baculovirus system (right). IFNgamma production is expressed in <<fold change>> considering untreated PBMC as control (value=1). Data are shown as Mean±SD of three independent experiments. *p<0.05, **p<0.01.

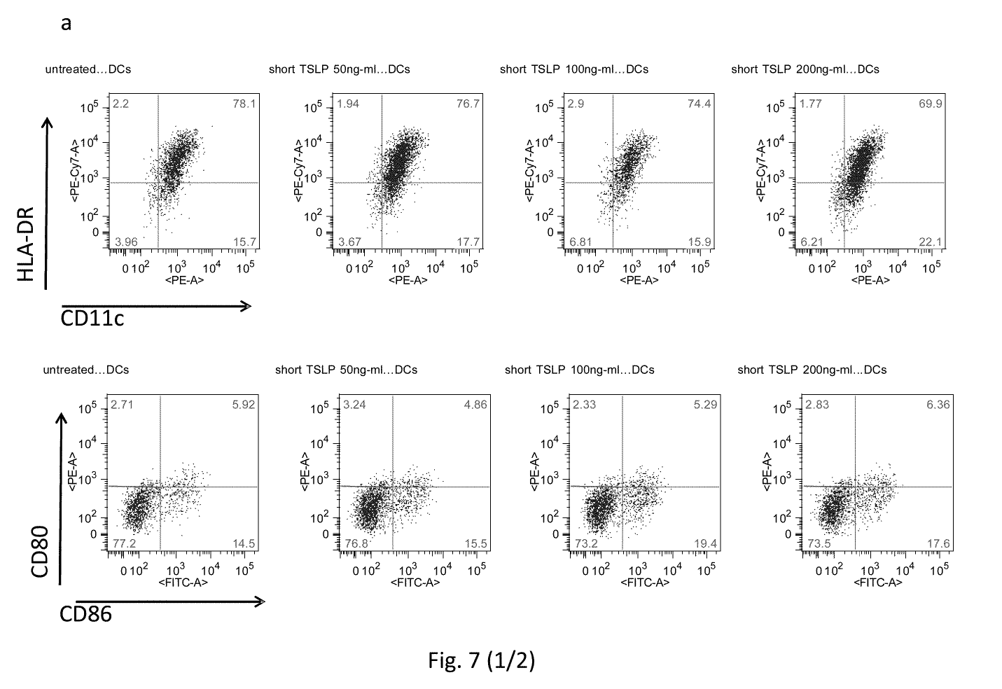

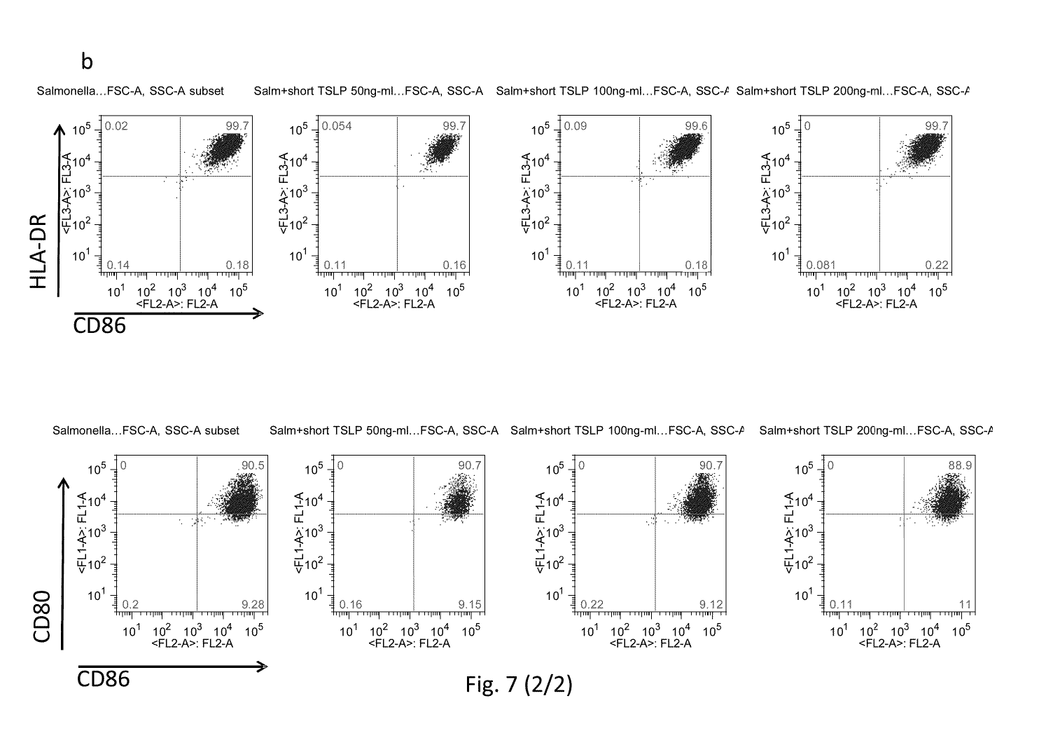

FIG. 7: Short TSLP does not change mature and immature DC activation status. FACS analysis of monocyte derived DCs conditioned with the indicated doses (50, 100 or 200 ng/ml) of short TSLP for 24 hours a) without or b) with subsequent challenge with Salmonella (MOI 1:1).

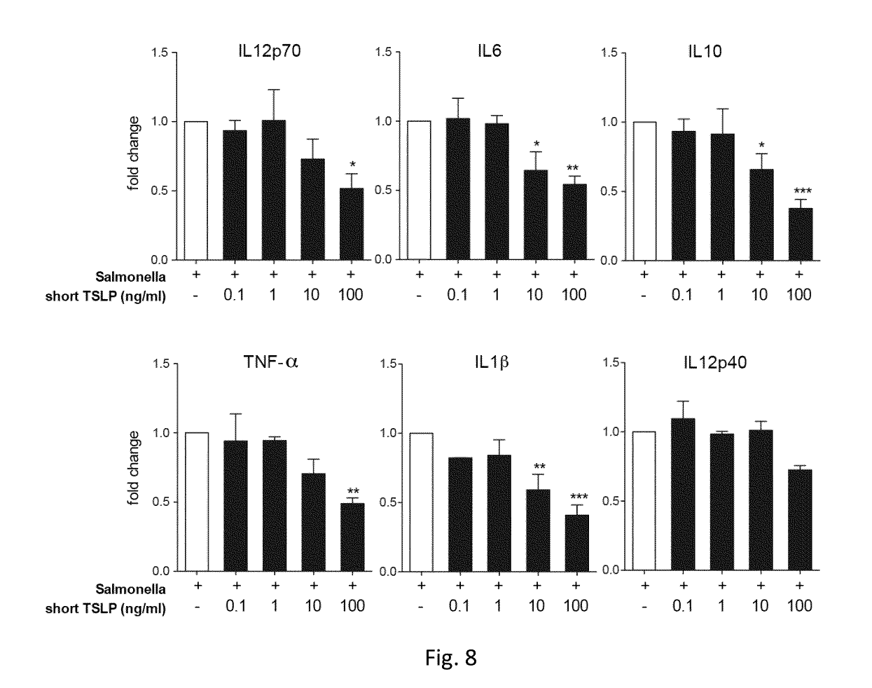

FIG. 8: Short TSLP inhibits dendritic cell cytokine secretion. Analysis of inflammatory cytokine secretion of monocyte-derived DCs differentiated from human peripheral blood. Cells were first conditioned for 24 hours with increasing doses of short TSLP and then infected with Salmonella (clone FB62) for 1 hour (ratio 1:1). Data are expressed in ‘fold change’ considering cells treated only with Salmonella as a control (value=1). Data are shown as Mean±SD of four independent experiments. *p<0.05, **p<0.01, ***p<0.001.

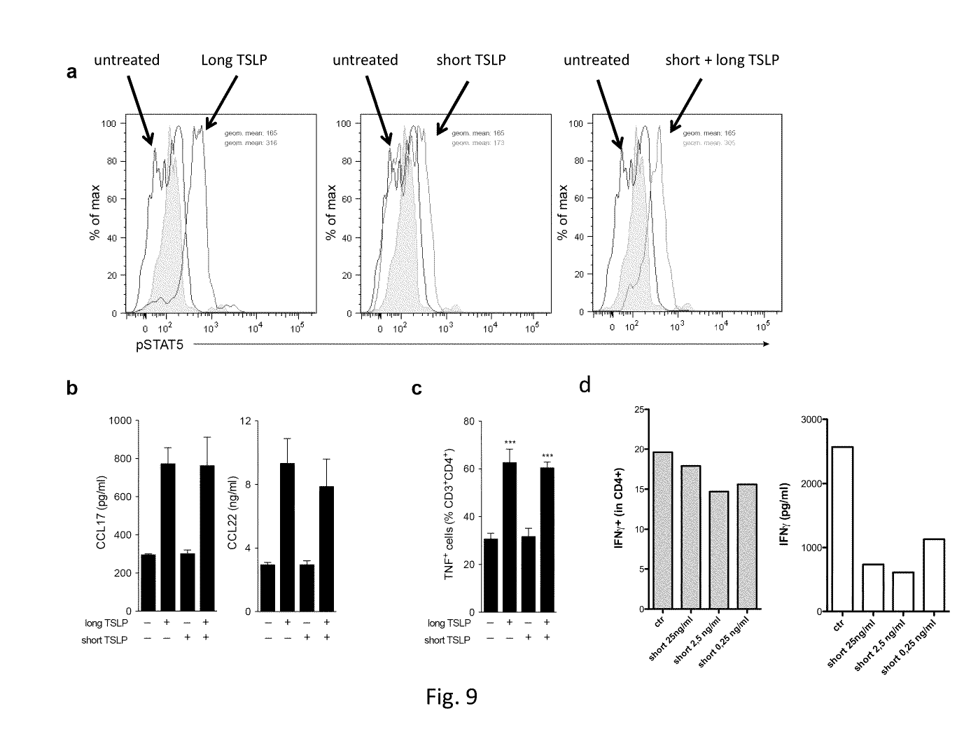

FIG. 9: Short TSLP does not interfere with long TSLP effects on mDCs and inhibits Th1 responses. (a) Flow cytometry assessment of human STATS pY694 on myeloid dendritic cells (mDC) isolated from PBMC. Freshly isolated cells were left untreated or incubated with an equimolar dose of short and/or long TSLP for 15 minutes before performing intracellular staining protocol with the anti-pSTAT5 antibody or the corresponding isotype (grey). (b) ELISA analysis of CCL17 and CCL22 levels in the supernatants of mDC treated as in (a) for 24 hours. Data are shown as Mean±SD of two independent experiments. (c) The same cells, after TSLP incubation, were washed and coculture with human naïve CD4 T cells from a different donor (ratio 1:5) for 6 days. Intracellular staining of T cells for TNF was performed after 4 hours restimulation with PMA (phorbol-12-myristate-13-acetate) and ionomycin. Data are shown as Mean±SD of two independent experiments performed with one donor of mDC and two donors of T cells. *** p<0.001. (d) mDC isolated from PBMC were left untreated or incubated with increasing doses of short TSLP for 24 h and then, after washing, co-cultured with human naïve CD4 T cells for 6 days as in (c). The graph shows a representative analysis of IFNgamma levels in the supernatant of the coculture (day 6) (right) and an intracellular staining for IFNgamma positive cells (left).

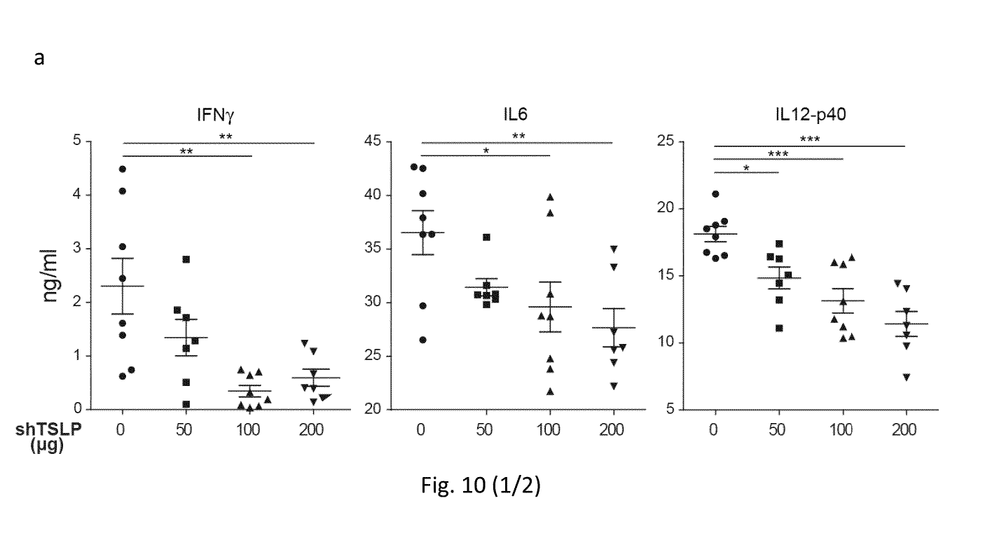

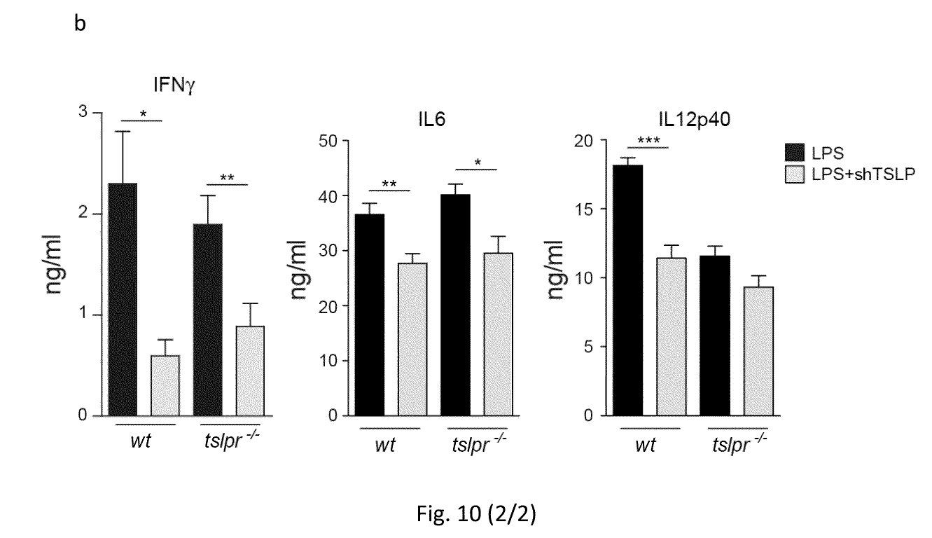

FIG. 10: Short TSLP dampens endotoxin-induced inflammatory effects in vivo in a TSLPR independent manner. (a) Analysis of inflammatory cytokine levels in the sera of mice treated i.p. with increasing doses of short TSLP. Short TSLP was injected twice, 12 hours and 2 hours, before inducing LPS shock (200 μg LPS). Blood was collected 6 hours later. (b) Cytokine levels in the sera of wt and tslpr−/− mice treated as in (a) with the higher dose of short TSLP (200 μg). Data are representative of two independent experiments. * p<0.05, ** p<0.01, *** p<0.001.

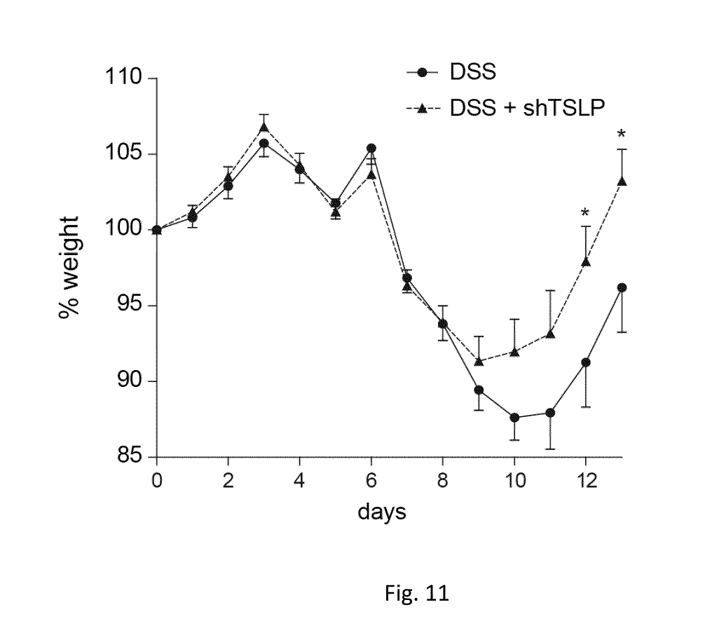

FIG. 11: Short TSLP protects mice from DSS-induced colitis. Body weight changes in mice with DSS-induced colitis treated every other day with short TSLP (200 ug) or the vehicle i.p. Graph shows percentage of body weight relative to initial body weight. * p<0.05

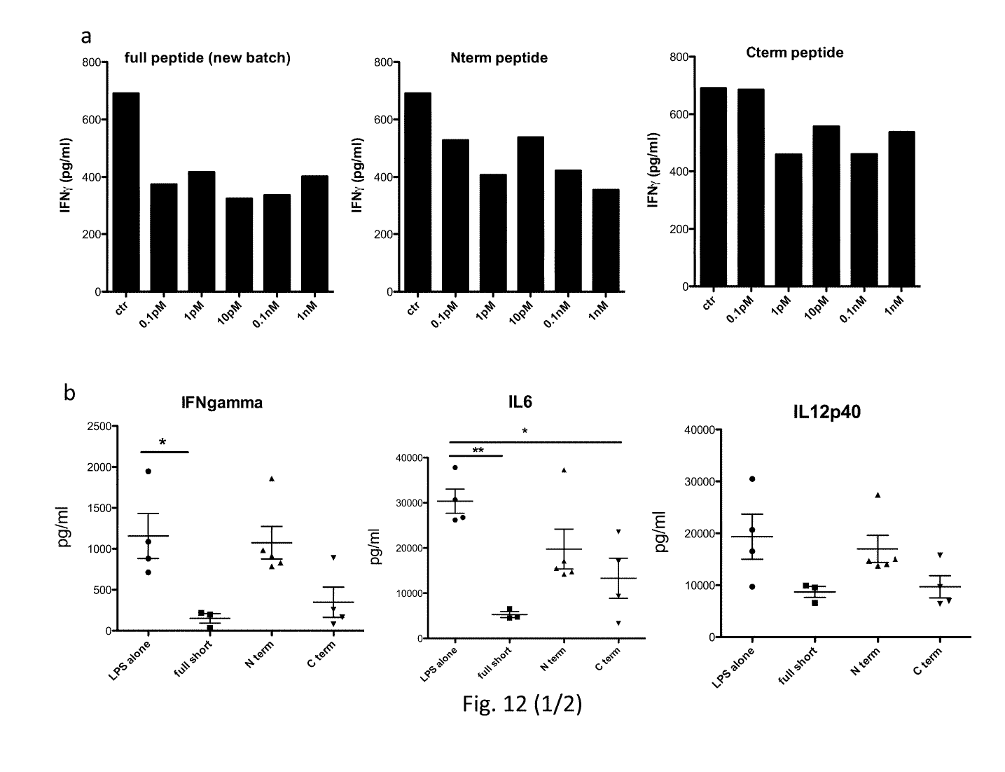

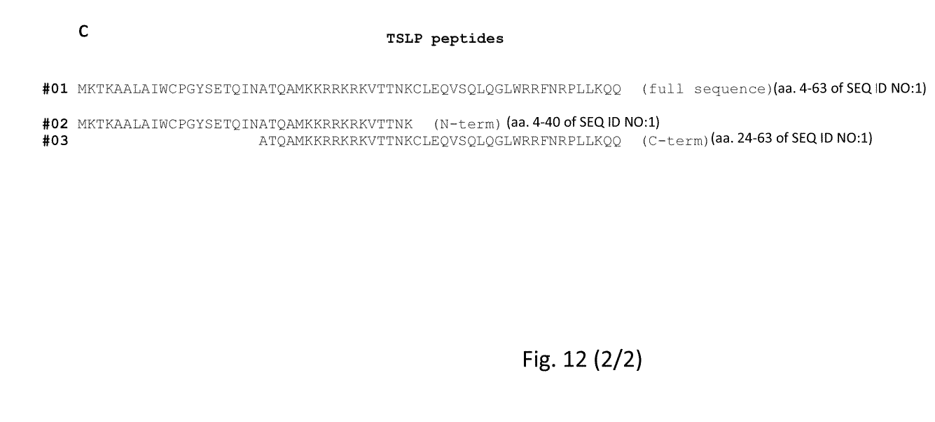

FIG. 12. (a) Quantification of IFNgamma production in a bidirectional MLR performed like in FIG. 6 with increasing doses of short TSLP, full peptide or N terminal (Nterm) and C terminal (Cterm) portion. IFNgamma production is expressed in ‘fold change’ considering untreated PBMC as control (value=1). Data are shown as Mean±SD of three independent experiments. * p<0.05, ** p<0.01. (b) Analysis of inflammatory cytokine levels in the sera of mice treated like in FIG. 10 with short TSLP peptides. (c) Aminoacids sequence of short TSLP (#01, corresponding to aa. 4-63 of SEQ ID NO:1) and two of its fragments starting either from the N terminal (#02, corresponding to aa. 4-40 of SEQ ID NO:1) or from the C terminal of the protein (#03, corresponding to aa. 24-63 of SEQ ID NO:1), with 17 aa of overlapping.

FIG. 13: Anti long-TSLP antibody has a blocking activity. ELISA analysis of CCL17 levels in the supernatants of mDC treated for 24 hours with long TSLP (50 ng/ml) in presence of increasing doses of anti-long TSLP antibody or isotype control.

FIG. 14: Long TSLP expression is increased in intestinal cells from ulcerative colitis (UC) patients compared to healthy individuals. Quantitative real-time PCR analysis of short and long TSLP mRNA in whole mucosal intestinal tissue from healthy (H) individuals and UC patients. The ‘fold induction’ in mRNA expression is in reference to that of the ‘housekeeping’ gene, Gapdh. *p<0.05.

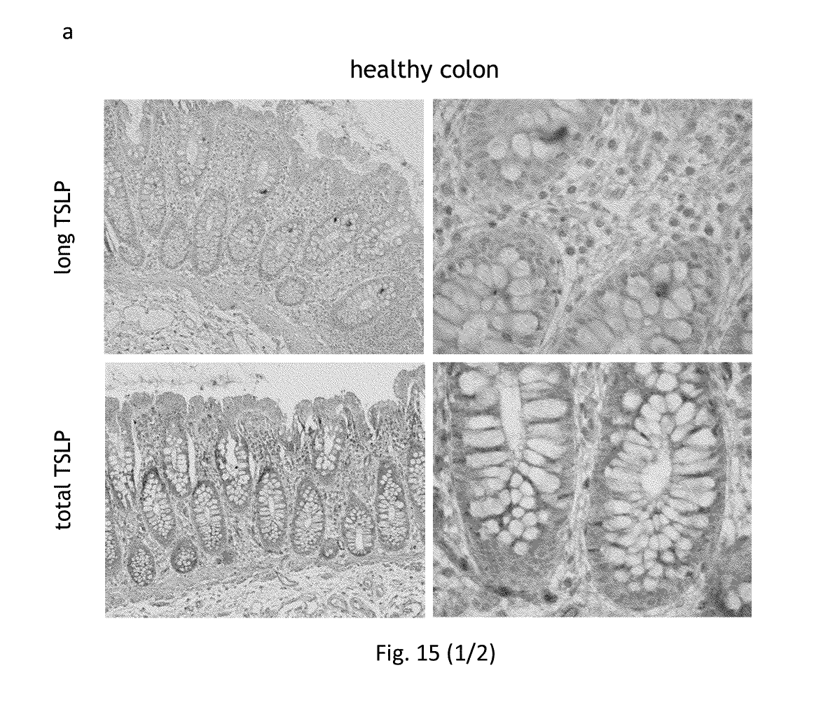

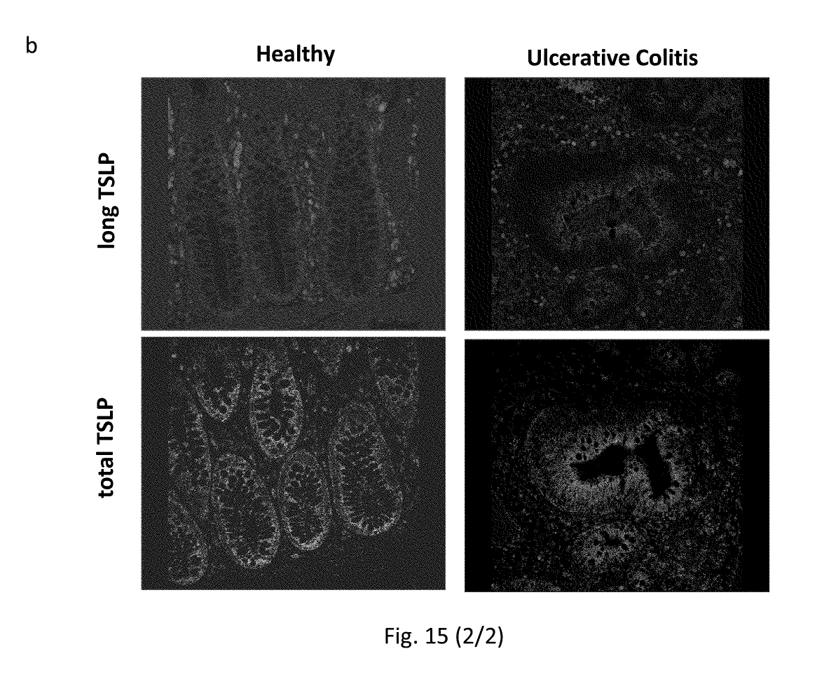

FIG. 15: Short and long TSLP are differentially expressed in the intestine at protein level under steady state or inflammation. Representative immunohistochemical (a) and immunfluorescent (b) stainings of long TSLP (upper panels) and both TSLP isoforms (total TSLP) (bottom panels) in sections of colon from healthy individuals (a, b) or from ulcerative colitis patients (b).

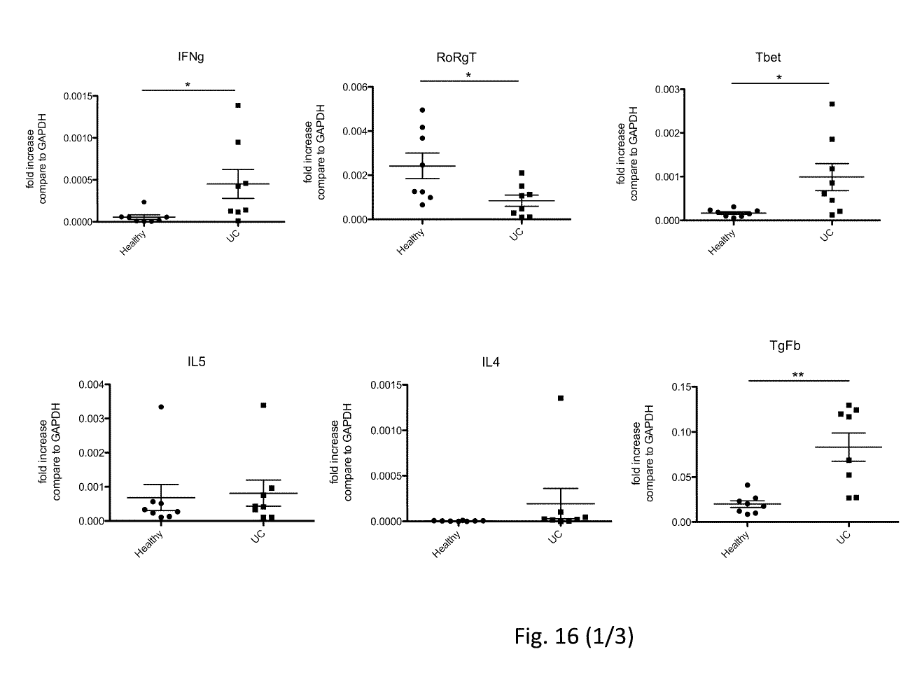

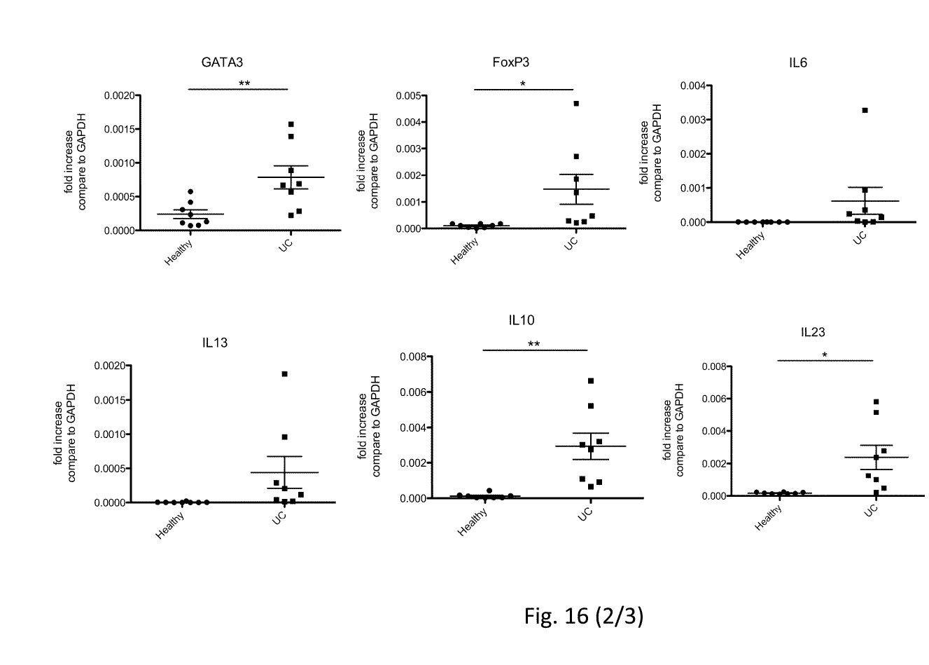

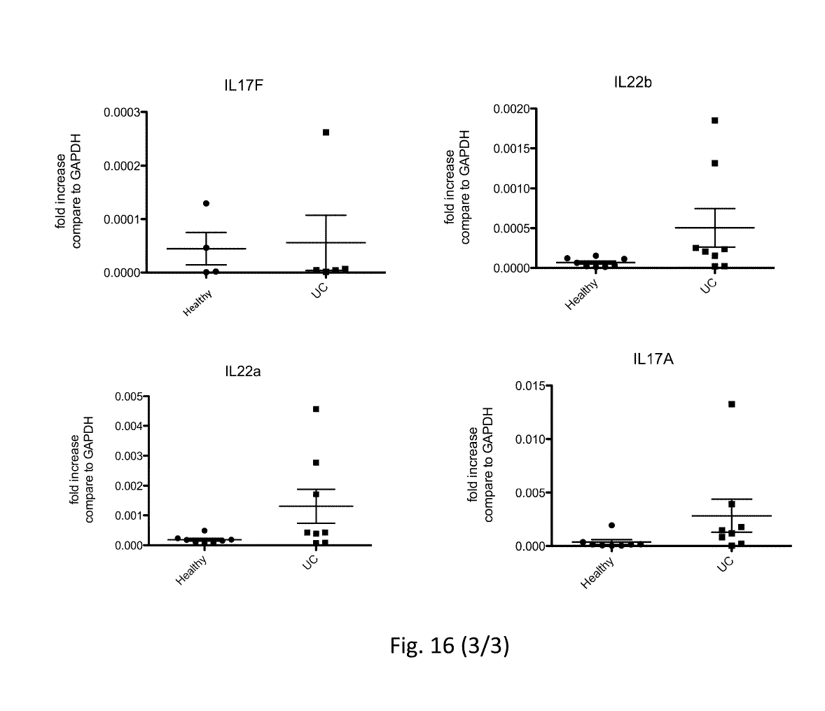

FIG. 16: Cytokine and transcription factor profiling of ulcerative colitis tissues. Quantitative real-time PCR analysis of different transcription factor and cytokine mRNAs in intestinal tissue from healthy individuals and UC patients. The ‘fold induction’ in mRNA expression is in reference to that of the ‘housekeeping’ gene, Gapdh. *p<0.05 **p<0.01

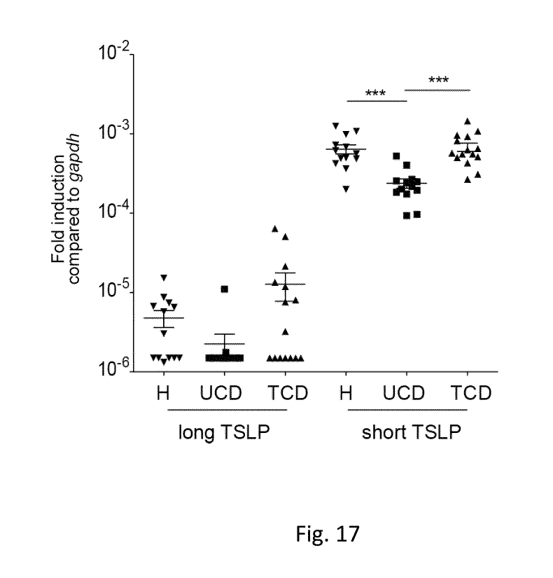

FIG. 17: Short TSLP expression is upregulated in intestinal tissue of coeliac patients after treatment. Quantitative real-time PCR analysis of short TSLP and long TSLP mRNA in intestinal tissue samples of untreated (UCD, n=13) and treated (TCD, patients having a gluten-free diet, n=15) celiac disease (CD) patients and of control healthy individuals (H, n=13). The ‘fold induction’ in mRNA expression is in reference to that of the ‘housekeeping’ gene, Gapdh. ***p<0.001

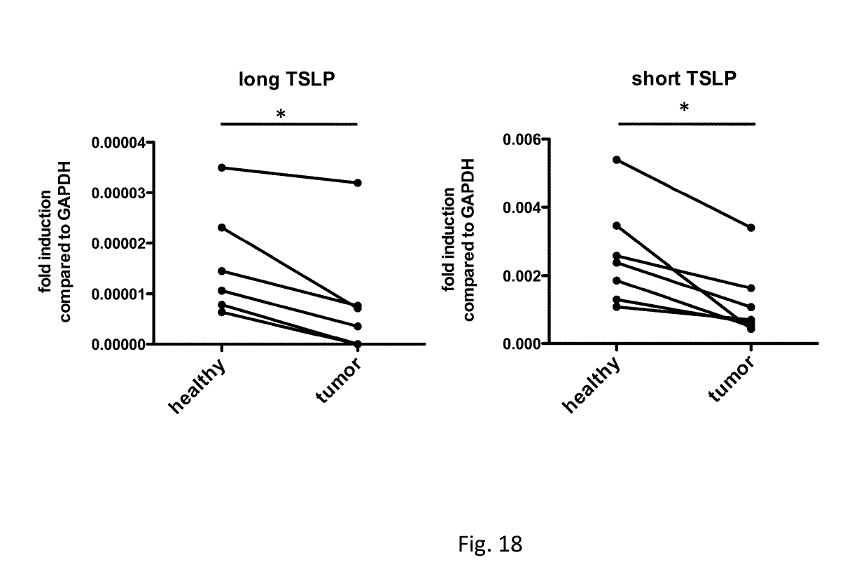

FIG. 18: Short and long TSLP are downregulated in neoplastic intestinal tissue compared to healthy counterpart. Quantitative real-time PCR analysis of long TSLP and short TSLP mRNA in healthy (at least 7 cm far from the neoplasy) and neoplastic colon tissue from colorectal cancer patients. The ‘fold induction’ in mRNA expression is in reference to that of the ‘housekeeping’ gene, Gapdh. *p<0.05

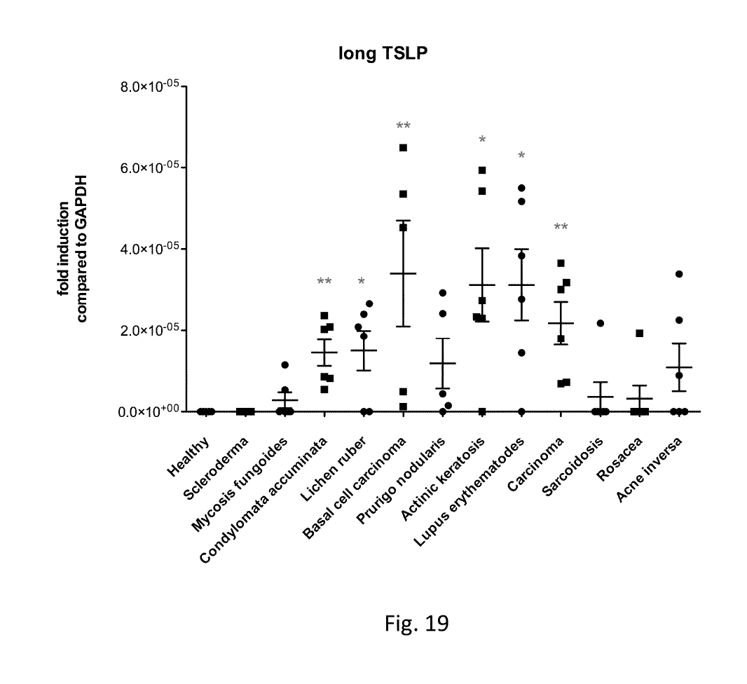

FIG. 19: Long TSLP expression is induced in different inflammatory disorders of the skin. Quantitative real-time PCR analysis of long TSLP mRNA in tissue samples from inflammatory skin disorder patients. The ‘fold induction’ in mRNA expression is in reference to that of the ‘housekeeping’ gene, Gapdh. *p<0.05, **p<0.001.

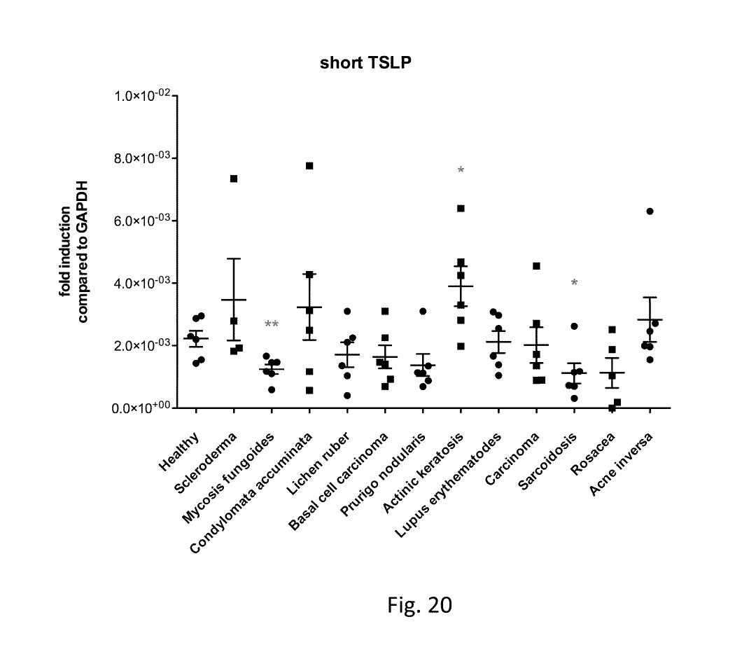

FIG. 20: Short TSLP expression is downregulated in different inflammatory disorders of the skin. Quantitative real-time PCR analysis of short TSLP mRNA in tissue samples from inflammatory skin disorder patients. The ‘fold induction’ in mRNA expression is in reference to that of the ‘housekeeping’ gene, Gapdh. *p<0.05, **p<0.001.

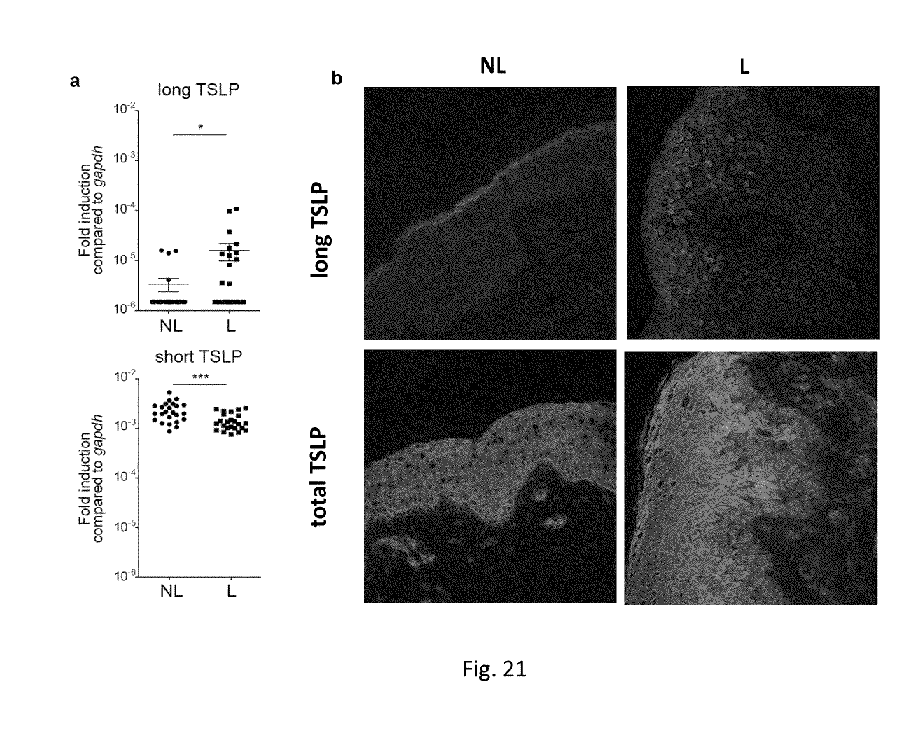

FIG. 21: Short and long TSLP are differentially expressed in skin tissue of atopic dermatitis patients. (a) Quantitative real-time PCR analysis of short and long TSLP mRNA in skin biopsies from non-lesional (NL) and lesional (L) atopic dermatitis patients. The ‘fold induction’ in mRNA expression is in reference to that of the ‘housekeeping’ gene, Gapdh. * p<0.05, *** p<0.001. (b) Representative immunofluorescent stainings of long TSLP (upper panels) and both TSLP isoforms (total TSLP) (bottom panels) in tissue sections of NL and L patients.

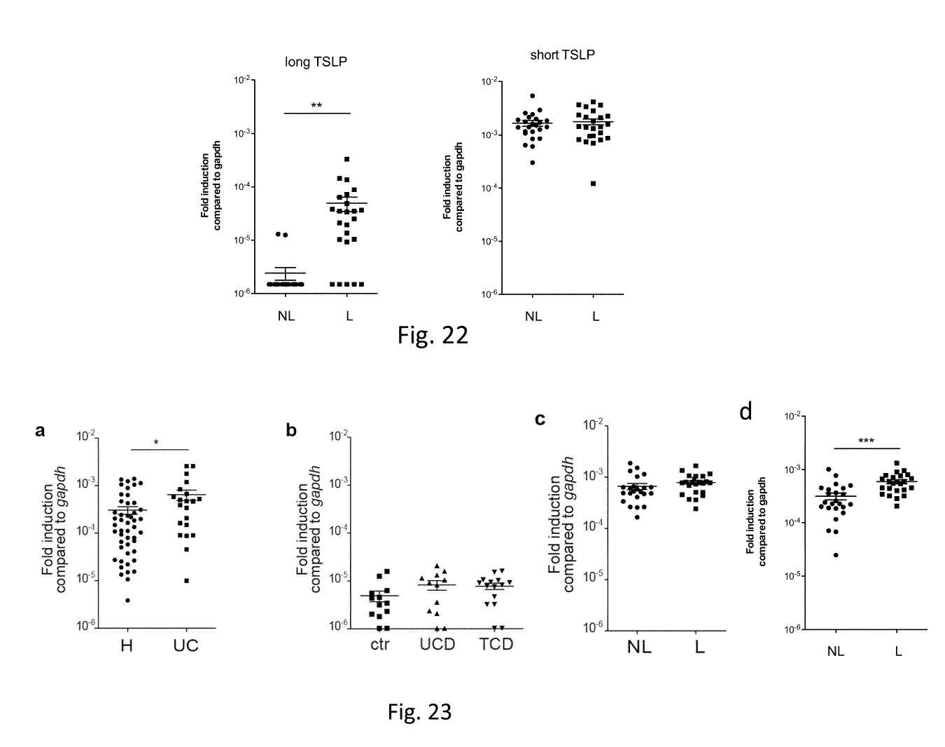

FIG. 22: Long TSLP expression is increased in skin tissue of psoriasis patients.

Quantitative real-time PCR analysis of short and long TSLP mRNA in skin biopsies from non-lesional (NL) and lesional (L) psoriasis patients. The ‘fold induction’ in mRNA expression is in reference to that of the ‘housekeeping’ gene, Gapdh. ** p<0.01.

FIG. 23: Quantitative real-time PCR analysis of TSLPR mRNA in intestinal cells from healthy individuals or UC patients (a); in intestine biopsies of healthy individuals (ctr) and coeliac patients (b); in skin biopsies of atopic dermatitis (c) and psoriasis (d) patients. The ‘fold induction’ in mRNA expression is in reference to that of the ‘housekeeping’ gene, Gapdh. * p<0.05, *** p<0.001. H: healthy, UC: ulcerative colitis, UCD: untreated coeliac disease, TCD: treated coeliac disease, NL: non lesional, L: lesional.

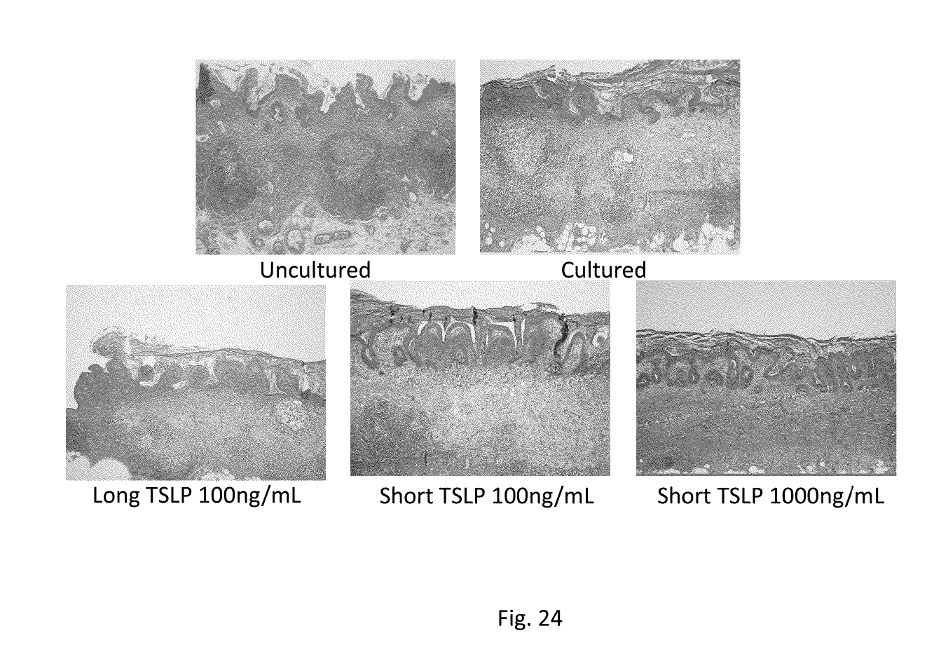

FIG. 24: Analysis of TSLP activity on an ex vivo organ culture model of the inflamed intestine. The tissue coming from surgical specimen of ulcerative colitis patients was separated in mucosal and submucosal layer. The mucosal layer was cultured overnight with TSLP isoforms on the apical and basolateral side of the tissue as indicated.

### DETAILED DESCRIPTION OF THE INVENTION

### Experimental Procedures

### Sequences

cDNAs encoding long form of human TSLP
(SEQ ID NO: 4):
ATGTTCCCTTTTGCCTTACTATATGTTCTGTCAGTTTCTTTCAGGAAAAT
CTTCATCTTACAACTTGTAGGGCTGGTGTTAACTTACGACTTCACTAACT
GTGACTTTGAGAAGATTAAAGCAGCCTATCTCAGTACTATTTCTAAAGAC
CTGATTACATATATGAGTGGGACCAAAAGTACCGAGTTCAACAACACCGT
CTCTTGTAGCAATCGGCCACATTGCCTTACTGAAATCCAGAGCCTAACCT
TCAATCCCACCGCCGGCTGCGCGTCGCTCGCCAAAGAAATGTTCGCCATG
AAAACTAAGGCTGCCTTAGCTATCTGGTGCCCAGGCTATTCGGAAACTCA
GATAAATGCTACTCAGGCAATGAAGAAGAGGAGAAAAAGGAAAGTCACAA
CCAATAAATGTCTGGAACAAGTGTCACAATTACAAGGATTGTGGCGTCGC
TTCAATCGACCTTTACTGAAACAACAGTAA
cDNAs encoding short form of human TSLP
(SEQ ID NO: 3):
ATGTTCGCCATGAAAACTAAGGCTGCCTTAGCTATCTGGTGCCCAGGCTA
TTCGGAAACTCAGATAAATGCTACTCAGGCAATGAAGAAGAGGAGAAAAA
GGAAAGTCACAACCAATAAATGTCTGGAACAAGTGTCACAATTACAAGGA
TTGTGGCGTCGCTTCAATCGACCTTTACTGAAACAACAGTAA
Amino acid sequence of the long form of human TSLP
(SEQ ID NO: 2)
MFPFALLYVLSVSFRKIFILQLVGLVLTYDFTNCDFEKIKAAYLSTISKD
LITYMSGTKSTEFNNTVSCSNRPHCLTEIQSLTFNPTAGCASLAKEMFAM
KTKAALAIWCPGYSETQINATQAMKKRRKRKVTTNKCLEQVSQLQGLWRR
FNRPLLKQQ (NCBI Accession n.
gi|14719428|ref|NP_149024.1| thymic stromal
lymphopoietin isoform 1 precursor [Homo sapiens])
Amino acid sequence of the short form of human
TSLP (SEQ ID NO: 1)
MFAMKTKAALAIWCPGYSETQINATQAMKKRRKRKVTTNKCLEQVSQLQ
GLWRRFNRPLLKQQ (also corresponding to aa. 97-159
of SEQ ID NO: 2; NCBI Accession n.
gi|190886449|ref|NP_612561.2| thymic stromal
lymphopoietin isoform 2 [Homo sapiens])
Fragment of the amino acid sequence of the short
form of human TSLP (corresponding to aa.4-63 of
SEQ ID NO: 1):
MKTKAALAIWCPGYSETQINATQAMKKRRKRKVTTNKCLEQVSQLQGLW
RRFNRPLLKQQ (NCBI Accession n.
gi|16876879|gb|AAH16720.1| Thymic stromal
lymphopoietin [Homo sapiens])

### Tissue Sampling

Intestinal mucosa was excised from the intestines of ulcerative colitis patients at the time of surgery. Healthy intestinal samples were obtained from the healthy tissue (at least 7 cm away from neoplastic tissue) of patients undergoing surgery for colon cancer. The healthy parts of the mucosal layer were separated from the rest of the tissue by a pathologist and directly transferred to our laboratory. Transfer was carried out in both cases in Hank's Balanced Salt Solution (HBSS) buffer supplemented with bacteriostatic antibiotics. The tissue was kept at 4° C. during the transfer. Mucosa from celiac disease patients was obtained in the form of biopsies during routine colonoscopy. Skin biopsies were taken from either healthy individuals undergoing plastic surgery or patients suffering from atopic dermatitis. The clinical diagnosis was confirmed by dermatopathological evaluation. All tissues were obtained from patients who signed an informed consent approved by the institutional review board, allowing material not required for diagnosis to be used for research purposes. The samples were handled in a completely anonymous manner, and they received a serial number by the pathologist for crosschecking histological reference.

### Isolation of Intraepithelial Cells (IECs) and Lamina Propria (LP) Cells

Upon arrival in the laboratory intestinal mucosa was processed for IECs and LP cells isolation as previously described [Matteoli G, et al. Gut 2010]. Briefly, the tissue was cut into small pieces and washed twice in HBSS 1% fetal bovine serum (Gibco), 1 mM DTT for 15 min at 37° C. to remove the mucus. IECs were then detached by washing with HBSS, 1% FBS, 1 mM EDTA for additional 15 min IECs were pelleted at 300 g for 10 min and lysed with Trizol reagent (Ambion, Invitrogen) for RNA extraction. The rest of the tissue was cut up in 1 mm2 pieces and digested in α-MEM with 5% FBS, in the presence of collagenase VIII (1 mg/mL, Sigma-Aldrich) and DNase I (5 U/mL, Roche Diagnostics) 100 IU/ml penicillin and 100 μg/ml streptomycin for 10 min at 37° C. by gentle shaking (Geem D, et al. J Vis Exp 2012). The digestion medium was centrifuged at 300 g for 10 minutes and the LP cells pellet was lysed in Trizol reagent.

RNA Extraction and qPCR

RNA was extracted using the RNeasy Micro Kit (Qiagen). RNA was reverse transcribed with oligo(dT) and ImProm-II™ Reverse Transcriptase (Promega). Quantitative real-time PCRs were performed with Fast SYBR Green PCR kit on the Applied Biosystems 7900HT Fast RT-PCR System (Applied Biosystems). Results were quantified using the 2-DCt method (Livak and Schmittgen, Methods, 2001). Gene expression was normalized to the expression of the housekeeping gene, gapdh.

Different pairs of primers were designed for the two genes:

LONG TSLP FW:
(SEQ ID NO: 5)
5′-CACCGTCTCTTGTAGCAATCG
LONG TSLP RV:
(SEQ ID NO: 6)
5′-TAGCCTGGGCACCAGATAGC
SHORT TSLP FW:
(SEQ ID NO: 7)
5′-CCGCCTATGAGCAGCCAC
SHORT TSLP RV:
(SEQ ID NO: 8)
5′-CCTGAGTAGCATTTATCTGAG

### Bacterial Strains

Salmonella enterica serovar typhimurium SL1344 strain FB62 and Escherichia coli strains LF82 and MG1655 were cultured in TB broth [kind gift from Arlette Darfeuille Michaud]. For infection, bacteria were grown overnight in 3 ml of TB at 37° C. in agitation. The following day 1 mL of pre-inoculum was added in 9 ml of fresh TB medium and was grown at 37° C. in agitation until reaching the exponential growth phase (O.D. between 0.55 and 0.65).

### Cell Culture

Caco2 cells were cultured in DMEM supplemented with 10% FBS, 1% Glutamine, 1% non-essential aminoacids, 1% Penicillin-Streptomycin. HaCaT cells were cultured in DMEM supplemented with 10% FBS, 1% Glutamine, 1% Penicillin-Streptomycin. For growth on transwell inserts (Corning), 2×10⁵ caco2 cells were seeded on 6 mm² transwells and grown for 10-12 days, until transepithelial resistance was roughly 650 mΩ. Cells were infected with bacteria in the absence of antibiotics at a multiplicity of infection of 1:100 for one hour. Then medium was replaced with complete medium containing 100 μg/mL Gentamicin and the cells were cultured for a further 23 hours. HaCaT cells were challenged with calcitriol, an active form of vitamin D, (10 μM, Enzo Life Sciences), or poly(I:C) (1 μg/mL) for 24 hours. At the end of the challenge cells were lysed in Trizol (Ambion, Life Technologies) reagent for RNA extraction or RIPA buffer for protein extraction.

### Reagents

TSLP recombinant protein, long TSLP (in a form having a sequence of aa. 29-159 of SEQ ID NO: 2, thereby not presenting the signal peptide of sequence of aa. 1-28 which is cleaved after translation; for the full protein: mw 15 KDa, Uniprot: Q969D9), was purchased from R&D Systems.

Cloning and Expression of the Two TSLP Isoforms and Fragment TSLP (fragTSLP) Antibody

The cDNAs encoding long and short forms of human TSLP were generated by PCR and subcloned into BamHI/SalI site of pFL-GST transfer vector (Invitrogen). The resulting baculovirus was used for expression in insect cells (Sf9 and Hi5 cells). Insect cells were harvested by centrifugation, resuspended in lysis buffer (20 mM Tris [pH 7.4], 300 mM NaCl, 5% glycerol, 1 mM EDTA, 1 mM DTT and protease inhibitors), sonicated gently and the lysates were cleared by centrifugation. TSLP fragments were absorbed to Gluthation Sepharose Beads (Amersham) and eluted using lysis buffer supplemented with 20 mM reduced Gluthation (Sigma). The eluted proteins were dialysed in buffer containing 20 mM Tris [pH 7.4], 200 mM NaCl, 5% glycerol, 1 mM EDTA, 1 mM DTT.

For anti-long TSLP specific antibody preparation, the fragment of the long isoform which is absent in the short (fragTSLP, corresponding to aa.1-96 of SEQ ID NO. 2) was cloned and expressed in E. coli cells, and purified with the same strategy (DsbA tag). Rabbits were immunized with the tagged fragment in the presence of incomplete Freund's adjuvant and the resulting sera were depleted of anti-DsbA antibodies.

### Short TSLP Peptides Synthesis

Short TSLP peptides were synthesized in house and resuspended in sterile water. Endotoxin levels were below 0.1 ng/μg of peptide as determined by the LAL test.

### Sequences:

short TSLP (aa 4-63 of SEQ ID NO: 1, mw 7 KDa, Uniprot: Q96AU7)
N terminal peptide (aa 4-40 of SEQ ID NO: 1, mw 4.2 KDa)
C terminal peptide (aa 24-63 of SEQ ID NO: 1, mw 4.8 KDa)

The peptides were assembled by stepwise microwave-assisted Fmoc-SPPS on a Biotage ALSTRA Initiator+peptide synthesizer, operating in a 0.12 mmol scale on a HMPB-ChemMatrix resin (0.45 mmol/g). Resin was swelled prior to use with a NMP/DCM mixture. Activation and coupling of Fmoc-protected amino acids was performed using Oxyma 0.5M/DIC 0.5M (1:1:1), with a 5 equivalent excess over the initial resin loading. Coupling steps were performed for 7 minutes at 75° C. Deprotection steps were performed by treatment with a 20% piperidine solution in DMF at room temperature (1×3 min+1×5 min) Following each coupling or deprotection step, peptidyl-resin was washed with DMF (4×5 ml). Following chain assembly, peptide was cleaved from the resin using a TFA 90%, water 5%, thioanisole 2.5%, TIS 2.5% mixture (3 hours, RT). Following precipitation in cold diethyl ether, crude peptide was collected by centrifugation and washed with further cold diethyl ether to remove scavengers. Peptides was then dissolved in 50% aqueous acetonitrile 0.07% TFA buffer and purified by preparative RP-HPLC.

### RP-HPLC Analysis and Purification:

Analytical and semi-preparative reversed phase high performance liquid chromatography (RP-HPLC) were carried out on a Tri Rotar-VI HPLC system equipped with a MD-910 multichannel detector for analytical purposes or with a Uvidec-100-VI variable UV detector for preparative purpose (all from JASCO, Tokyo, Japan). A Phenomenex Jupiter 5μ C18 90 Å column (150×4 6 mm) was used for analytical runs and a Phenomenex Jupiter 10μ C18 90 Å (250×21.2 mm) for peptide purification. Data were recorded and processed with Borwin software. 2%/min linear gradient of 0-60% eluent B (eluent A=H₂O/3% CH₃CN/0.07% TFA, eluent B=70% CH₃CN/30% H₂O/0.07% TFA) was employed at a flow rate of 1 mL/min for analytic purposes. UV detection was recorded in the 220-320 nm range. Peptide purification was achieved by preparative RP-HPLC at a flow rate of 14 mL/min using a 100% A→30% B gradient over 40 min Pure RP-HPLC fractions (>95%) were combined and lyophilized.

### Reagents:

HMPB-ChemMatrix resin and N-a-Fmoc-L-amino acids used during chain assembly were purchased from his Biotech GmbH (Marktredwitz, Germany). Ethyl cyanoglyoxylate-2-oxime (Oxyma) was purchased from Novabiochem (Darmstadt, Germany), N,N′-dimethylformamide (DMF) and trifluoroacetic acid (TFA) were from Carlo Erba (Rodano, Italy). N,N′-diisopropylcarbodiimide (DIC), dichloromethane (DCM) and all other organic reagents and solvents, unless stated otherwise, were purchased in high purity from Sigma-Aldrich (Steinheim, Germany). All solvents for solid-phase peptide synthesis (SPPS) were used without further purification. HPLC grade acetonitrile (ACN) and ultrapure 18.2Ω water (Millipore-MilliQ) were used for the preparation of all buffers for liquid chromatography. The chromatographic columns were from Phenomenex (Torrance Calif., USA)

### Endotoxin Contamination Assay

Endotoxin contamination was assessed using the Limulus amebocyte lysate (LAL) assay. (Lonza, #50-647U)

### Peripheral Blood Mononuclear Cells Mixed Lymphocyte Reaction

Buffy coats were obtained from healthy donors (Abbiategrasso hospital, Italy) with informed consent for research use. Peripheral blood mononuclear cells (PBMC) were separated with Ficoll (GE Healthcare) gradient centrifugation and then resuspended and cultured in RPMI 1640 medium (Lonza) containing 10% fetal bovine serum (Gibco), 1% Glutamine 1% pyruvate, 1% non essential AA and 1% Penicillin-Streptomycin. PBMC from two different donors were co-cultured (1:1) in presence of long and short TSLP at different concentrations. After 5 days supernatants were collected and IFNgamma levels were measured by ELISA (R&D systems).

### Monocyte-Derived Dendritic Cell Differentiation and Stimulation Conditions

Peripheral CD14⁺ monocytes were isolated from PBMC using human CD14⁺ microbeads (Miltenyi Biotec) according to the manufacturer's instructions. Cells were then cultivated in complete RPMI 1640 medium in the presence of IL4 (2.5 ng/ml) and GM-CSF (5 ng/ml) (BD Biosciences) for 6 days to obtain monocyte-derived dendritic cells. The percentage and the phenotype of cell subsets were evaluated before and after differentiation by flow cytometry. Dendritic cells were seeded in complete RPMI 1640 medium at 1×10⁶/ml in 48-well plates in the presence of short TSLP at different concentrations or culture medium alone. After 24 hours of culture dendritic cells were gently washed and challenged with Salmonella typhimurium FB62 (MOI 1:1) for 1 hour. Bacteria were then washed out and the medium replaced with medium containing 100 μg/ml gentamycin. Cytokine secretion was assessed after 24 hours by cytometric bead assay Flex sets (BD Biosciences). CBA were acquired on a BD Accuri C6 (BD Biosciences) and analyzed with FCAP software (BD Biosciences).

### Myeloid Dendritic Cell Isolation and Stimulation Conditions

Myeloid BDCA1+ cells were isolated from PBMC using CD1c (BDCA-1)+ Dendritic Cell Isolation Kit (Miltenyi Biotec) according to the manufacturer's instructions. Cells were then cultivated in complete RPMI medium in presence of long TSLP or short TSLP at the indicated doses, for 24 hours. Cells were then washed and cocultured with naïve CD4 T cells from a different donor (ratio 1 DC: 5 T). CD4 cells were isolated using naive CD4+ T Cell Isolation Kit II (Miltenyi Biotec) according to the manufacturer's instructions. After 6 days supernatants were collected and cells were restimulated for 4 hours with PMA (phorbol 12-myristate 13-acetate, 50 ng/ml) and ionomycin (1 ug/ml); for the last 3 hours brefeldin A (10 ug/ml) was added to the culture. Intracellular staining for TNF and IFNgamma positive cells was performed afterwards. CCL17, CCL22 and IFNgamma levels were assessed in the supernatant of DC or of the DC-T culture by ELISA (R&D Systems).

### Flow Cytometry

Cells were stained with monoclonal anti-human fluorescently conjugated antibodies: CD14 (M5E2), CD19 (HIB19), CD11c (B-ly6), CD45RA (HI100), CD3 (UCHT1), TNF (Mab11), IFNg (4S.B3), TNF (Mab11) from BD Biosciences; CD4 (RPA-T4) from Ebioscience; CD1a (HI149) and TSLPR (1B4) from Biolegend.

Myeloid cells isolated from PBMC were stimulated in complete RPMI 1640 (10⁶ cells/condition) with an equimolar dose of short TSLP and/or long TSLP (3.5 nM) for 15 minutes at 37° C. Control conditions included cells either left unstimulated (negative control) or incubated with sodium pervanadate (Na₃VO₈, positive control, data not shown). At the end of incubation cells were rapidly spun, fixed with BD Cytofix™ Buffer for 15 minutes at 37° C. and permeabilized with BD™ Phosflow Perm Buffer III on ice for 30 minutes, followed by intracellular staining using mouse anti-pSTAT5 (pY694) antibody (45/Stat5, BD Biosciences) or isotype antibody (MOPC-21). Samples were acquired on a BD Cantoll flow cytometer (BD Biosciences) and analyzed with FlowJo software (v8.7, Tree Star).

### Mice

Female C57Bl/6J mice (8 to 10 weeks of age) were obtained from Charles River Laboratories (Milan, Italy). TSLPR-deficient mice on the C57Bl/6J background were provided by Dr W. J. Leonard (Laboratory of Molecular Immunology, NHLBI, USA). Mice were bred and maintained at IFOM-IEO campus animal facility under specific pathogen-free conditions. All experiments were performed in accordance with the guidelines established in the Principle of Laboratory Animal Care (directive 86/609/EEC).

### LPS-Induced Endotoxic Shock

Mice were treated intraperitoneally (i.p.) with short TSLP at 50, 100, 200 μg per mouse in 200 μl of injectable water twice, 12 hours and 2 hours, before LPS administration (n=8 per group). Control mice received water. LPS (E. coli serotype 026:B6; Sigma-Aldrich) was injected intraperitoneally (i.p.) at 200 μg per mouse in 200 μl of injectable water. After 6 hours mice were euthanized by exsanguination under anesthesia and blood was collected. IFNgamma, IL6 and IL12-p40 levels were detected in the serum by ELISA (R&D Systems), according to manufacturer's instructions.

### DSS Colitis

Colitis was induced by adding 3% (w/v) DSS (TdB Consultancy AB, Uppsala, Sweden) to drinking water for 9 days. Mice were treated i.p. with short TSLP (200 μg in 200 μl of injectable water) the day before DSS administration and every other day during the entire study. Mice were monitored daily for weight loss.

### Immunofluorescence and Immunohistochemistry

Healthy and IBD mucosa was fixed in Hollande's fixative (Polysciences Inc.) and paraffin-embedded in a Leica ASP300 tissue processor. 5 μm-thick sections were deparaffinized and rehydrated and antigen unmasking (Vector laboratories solution) was performed at 95° C. for 50 minutes.

AD biopsies were included in OCT. 5 μm-thick sections were thawed for 5 minutes at room temperature and fixed for 15 minutes in 4% PFA. Sections were washed 3 times with TBS.

All sections were blocked in TBS-Tween 0.05% containing normal donkey serum. Primary antibodies were incubated overnight at 4° C. in the following concentrations: anti-TSLP (Abcam) 3 μg/mL; anti-longTSLP (in-house) 3.25 μg/mL; anti-CD14 (Abnova) 5 μg/mL. Secondary antibodies (donkey anti-rabbit 555 and donkey anti-mouse 488, Molecular Probes) were incubated for 1 hour and sections were counterstained with DAPI. Slides were mounted with Vectashield (Vector laboratories) and visualized under a Leica SP2 confocal microscope. For IHC, endogenous peroxidases were quenched with 0.3% H₂O₂ in methanol. Secondary antibody (P0448) and DAB complex (K3468) were purchased from DAKO and used according to the provider's instructions.

### Ex-Vivo Organ Culture

The activity of short TSLP on IBD tissues was assessed directly on human intestinal mucosa. Briefly, IBD mucosa was separated from the submucosa and mounted as described (Tsilingiri K., et al. J Vis Exp. 2013). Short or long TSLP was then incubated for 24 hours on the apical side and basolateral side in a final volume of 20 uL at the indicated concentrations (10, 100 and 1000 ng/mL).

### Results

### Expression of Short Form TSLP in Healthy Tissue is Constitutive

The inventors carried out an analysis on the UCSC Genome Browser of TSLP isoforms. The inventors found that there are three different isoforms annotated in RefSeq, two long and a short one. However, of the long ones only one seems to be a coding gene. Hence, the inventors focused on the canonical TSLP transcript variant 1 for the long isoform (NCBI Accession n. NM_{—}033035_hg19 160 chr5:110407589-110411772) and on transcript variant 2 (NCBI Accession n. NM_{—}138551_hg19 64 chr5:110409281-110411772) for the newly identified short isoform. The two transcripts code respectively for a long isoform of 159 aa and for a short isoform of 63 aa that is identical to the C terminus portion of long TSLP (FIG. 1). They are not alternatively spliced isoforms, but derive from the activity of two putative independent promoters (FIG. 1).

Even though the most well characterized form of TSLP so far is the long one, the inventors found the putative promoter for the long isoform to be almost completely inactive in most of the cell lines present in the UCSC database (FIG. 1). On the contrary, the promoter region for the short isoform seems to have a high capacity to bind a number of different transcription factors. Consistently, when the inventors performed qPCR on whole healthy intestinal and skin biopsies using specific primers for each isoform, the inventors found that short TSLP seems to be expressed at steady-state while the long isoform is often very little expressed when compared to the house-keeping gene GAPDH used as reference (FIG. 2 a, b).

Since the inventors have previously described an important homeostatic role for intestinal epithelial-cell derived TSLP in controlling the inflammatory potential of dendritic cells (DCs) (Iliev et al., 2009; Rimoldi et al., 2005), the inventors analyzed by qPCR the differential expression of the two isoforms in epithelial cells and lamina propria cells isolated from the healthy tissue of colon cancer patients (at least 7 cm from the neoplasy). Again, the inventors found that under steady-state intestinal epithelial cells and lamina propria cells express primarily the short isoform (FIG. 2a). This suggested that short TSLP may be responsible for the homeostatic activities of TSLP in the intestine. Hence, the inventors decided to clone and express the two isoforms of TSLP in an insect system (baculovirus-driven) so to avoid endotoxin contamination and to allow for proper glycosylation of the protein. While the expression of long TSLP was easily achieved, that of short TSLP was obtained only as a fusion protein with GST (FIG. 3). For this reason they also synthesized chemically the entire short TSLP peptide (aa 4-63) and two fragments of the short isoform, the N terminal peptide (aa 4-40) and the C terminal peptide (aa 24-63). Endotoxin contamination was assessed using the Limulus amebocyte lysate (LAL) assay and was found to be below the detection limit at the working concentration (not shown).

Epithelial Cells and Keratinocytes Upregulate Expression of Long TSLP after Challenge with Pro-Inflammatory Stimuli

In the gut it has been shown that mediators produced by IECs in response to the bacteria present in the lumen condition underlying immune cells, thus playing an active role in the shaping of both innate and adaptive immune responses. Hence, the inventors wondered whether they could observe a differential modulation of TSLP isoforms by IECs after bacterial challenge. To address this question, they first assessed the basal levels of the two isoforms' production by caco-2 cells, where they noticed an identical trend to the one they observed in primary intestinal epithelial cells (FIG. 4a). In order to confirm this data at the protein level, a polyclonal antibody targeted against the fragment of long TSLP that is not present in short TSLP was purified and tested in their laboratory (FIG. 3). They used this antibody to specifically detect the long isoform, while for the short one they used a commercially available antibody (Abcam). Western blot data on non-infected caco-2 cells confirmed the data obtained by rt-PCR (FIG. 4b). This trend however, was completely reversed when polarized caco-2 cells were challenged with an invasive strain of Salmonella, FB62 (FIG. 4c). The same was true when they used an invasive E. coli strain, LF82. Interestingly, its non invasive counterpart, MG1655, was not able to upregulate the long isoform. Finally, they examined the effect of pro- and anti-inflammatory stimuli in the two isoforms expression on a skin keratinocyte cell line, specifically the HaCaT cell line, which also does not express long TSLP in steady state (FIG. 5a). They challenged HaCaT cells with either vitamin D or polyI:C, a TLR3 agonist. The anti-inflammatory agent, namely vitamin D, significantly upregulated mRNA for short TSLP, while polyI:C upregulated the long isoform (FIG. 5b). Thus, they can conclude that IECs and skin ECs only express long TSLP upon detecting invasive bacteria or pro-inflammatory stimuli respectively.

### Short TSLP Inhibits a Bi-Directional Allogenic Mixed Leukocyte Reaction (MLR)

Short TSLP was used to assess whether it could inhibit the production of IFN-gamma in an allogenic MLR. Peripheral Blood Mononuclear Cells (PBMC) from two different donors were co-incubated in the presence of increasing doses of short or long TSLP. The inventors found that while long TSLP was actually increasing the amount of IFN-gamma released independent from the source (whether it was the commercial available source or the in-house produced protein), short TSLP was inhibiting the release of IFN-gamma by 50% (FIG. 6). This indicates that short TSLP can have anti-inflammatory properties.

### Short TSLP has Anti-Inflammatory Activity on MoDCs.

Having observed that short TSLP affects the production of IFN-gamma the inventors analyzed whether the effect was direct on dendritic cells, immune cells specialized in antigen presentation and T cell activation. Short TSLP was used to treat monocyte derived (Mo)DCs in the presence or absence of an additional inflammatory stimulus (Salmonella typhimurium). MoDCs were first incubated with short TSLP for 24 h and subsequently treated or not with bacteria (equivalent of Multiplicity Of Infection, MOI, 1). MoDCs treated with short TSLP did not display any functional or morphological activation as shown by no change in the expression of maturation markers CD80, CD86 or HLA-DR on both immature and Salmonella treated (mature) DCs, (FIG. 7a, b). CD11c is a marker characterizing dendritic cells. However, after LPS or bacterial stimulation short TSLP-treated DCs were strongly affected in their capacity to release pro-inflammatory as well as anti-inflammatory cytokines (TNF-alfa, IL12-p70, IL-10, IL-1beta, IL-6, FIG. 8).

Short TSLP does not Act as an Antagonist of Long TSLP on Myeloid DCs.

To assess the possibility of short TSLP binding to TSLPR and acting as an antagonist, blocking signal from the long isoform, myeloid DCs were conditioned for 15 minutes (FIG. 9a) or 24 hours (FIG. 9b) with the two isoforms, alone or together in an equimolar ratio. Long TSLP significantly increased STATS phosphorylation (FIG. 9a) and the secretion of CCL17 and CCL22 (FIG. 9b), that are hallmarks of long TSLP mediated signaling, both in the absence and in the presence of the short isoform. Moreover, long TSLP conditioned DCs promoted the differentiation of CD4+TNF+ cells, even in the presence of short TSLP. Thus, it appears that the short isoform does not mediate a counter effect to the long isoform (FIG. 9c). Interestingly, in the same set of experiments, they found that short TSLP conditioned DCs were capable of reducing the percentage of Th1 cells, as well as IFN-gamma secretion (FIG. 9d). This indicates that short TSLP can act on myeloid DCs inhibiting its inflammatory properties, but does not act via TSLPR and hence does not block those activities that are receptor dependent.

### Short TSLP has an Anti-Inflammatory Effect In Vivo.

The anti-inflammatory effect of the short TSLP was also documented in vivo, using a model of endotoxin shock in C57/BL6 mice. Mice were treated intraperitoneally with short TSLP 12 hours and 2 hours before being injected with LPS. Six hours after LPS injection, the mice were sacrificed and cytokines were measured in the sera. Short TSLP treatment led to a significant decrease of IL-6, IL-12p40 and IFN-γ in a dose-dependent manner (FIG. 10a). Of note, the anti-inflammatory effect of short TSLP was also manifested in TSLPR knock-out mice (FIG. 10b), further enhancing the hypothesis for a TSLPR independent mechanism of action for short TSLP. Finally, short TSLP also protected mice from DSS-induced colitis, significantly limiting weight loss, and promoting their recovery (FIG. 11).

### Effect of Short TSLP Fragments In Vitro and In Vivo.

With the perspective of using short TSLP as a therapeutic tool, the inventors tried to find the minimal active sequence. They designed two fragments of short TSLP starting either from the N terminal or from the C terminal of the protein, with 17 aa of overlapping (FIG. 12c). They tested both fragments in vitro, in a bi-directional allogenic MLR, and in vivo with the endotoxin shock model described in FIG. 10. Both peptides had an effect on human PBMC, inhibiting IFNgamma secretion (FIG. 12a). In the mouse system, instead, the C terminal peptide was able to reduce the level of inflammatory cytokines in the serum, similarly to the entire short TSLP, but the N terminal did not exert any effects (FIG. 12b). However, since the N-terminal was shown to be effective in vitro, the expert in the art knows that it is very likely that it will be effective in human or other organisms.

### Custom-Made Anti-Long TSLP Antibody Blocks the Activity of Long TSLP.

The two TSLP isoforms has completely opposite effects, the long one is inflammatory and deleterious while the short one seems to inhibit the development of inflammatory responses. The inventors demonstrated that short TSLP administration has beneficial effects but a parallel therapeutic strategy could involve the use of a blocking antibody specific for the long TSLP. Therefore they tested their custom-made anti-long TSLP antibody for its blocking activity. They stimulated myeloid DCs with long TSLP in the presence of increasing doses of anti-TSLP antibody or an isotype control and after 24 hours they quantified the level of CCL17 in the supernantant. Both antibodies inhibited the secretion of CCL17 but the anti-long TSLP effect was more striking compared to isotype control (FIG. 13). This preliminary experiment suggests that their custom antibody can be used as a blocking agent for long TSLP.

### Inverse Expression of Short/Long Isoforms in Ulcerative Colitis (UC) Patients

Having shown that short TSLP is the only isoform expressed under steady-state by intestinal epithelial cells (IECs) and having shown its anti-inflammatory potential, the inventors wanted to assess whether there was a deregulation of short TSLP expression in inflammatory bowel disease (of which UC is an example). qPCR analysis on IECs and lamina propria cells isolated from healthy or UC samples shows a significant upregulation of the long isoform, while the short one was not modified (FIG. 14). The same result was confirmed at the protein level by immunohistochemistry and immunofluorescence (FIG. 15a, b). Furthermore they found, in cells from UC tissue, an increased expression of TSLPR, the receptor for the long TSLP isoform (FIG. 23).

Long TSLP Expression is Associated with a Strong Inflammatory Response.

As the authors found that short TSLP inhibits the development of Th1 type of reactions, they assessed whether in the absence of short TSLP expression there was a skewing towards a more inflammatory response. In addition, as long TSLP is associated to Th2 responses, the inventors analyzed whether UC patients were characterized by a Th1 and Th2 biased response. The inventors performed qPCR on a set of genes for transcription factors and cytokines traditionally involved in inflammation. The inventors found that indeed in UC patients there was an increase in Th1 type of responses (as attested by an upregulation of IFN-gamma expression as well as T-bet, a critical regulator of the Th1 differentiation program, FIG. 16). In addition, the inventors observed a statistically significant upregulation in the expression of a transcription factor (GATA3) involved in Th2 skewing and an increase in cytokines involved in Th2 type of responses (IL-4, IL-13) even though the inventors could not detect statistical significance (FIG. 16). A bivariate fit analysis performed on pooled data from these experiments directly related the presence of the long isoform with upregulated IFN-gamma expression, and this was not the case for the short isoform.

### Proof of Concept Experiments on Non Th2-Related Diseases

In order to further validate the present results, the inventors assessed the expression of the two isoforms also in non Th2-related intestinal pathologies, such as coeliac disease and cancer. The inventors found that also in coeliac disease the short TSLP isoform was strongly down-regulated in untreated patients, while its levels were restored in treated patients (i.e. patients in a gluten-free diet, see FIG. 17). Of note, the inventors could not detect any increase in long TSLP or in TSLPR (FIG. 17, 23) expression and this is consistent with what has been described in the literature, namely that coeliac disease is predominantly a Th1 pathology.

The inventors found a deregulation of TSLP expression also in cancer, but in this case both isoforms were reduced in neoplastic tissue compared to their normal counterpart (FIG. 18). However, the expression of short TSLP still remains much higher than that of long TSLP. Finally, the inventors tested the expression of short TSLP in inflammatory disorders of the skin. Similarly to the intestine, the inventors found that only the short isoform is expressed under steady-state (FIG. 19-22) and that long TSLP is upregulated under inflammation like in condylomata accuminata, lichen ruber, basal cell carcinoma, actinic keratosis, lupus erythematodes and carcinoma (FIG. 19). Short TSLP levels remained unchanged or were even downregulated in mycosis fungoides and sarcoidosis (FIG. 20). Focusing on atopic dermatitis and psoriasis patients (FIG. 21, 22) they found an upregulation of the long TSLP, both at mRNA and at protein level, and for atopic dermatitis a significant decrease of short TSLP mRNA. TSLPR was greatly upregulated in biopsies from psoriasis patients (FIG. 23).

### Activity of Short TSLP on an Ex-Vivo Model of Intestinal Organ Culture (EVOC).

The inventors tested the activity of short and long TSLP on an ex-vivo organ culture model set up in their laboratory (Tsilingiri K, et al. J Vis Exp. 2013). The mucosal layer of intestinal tissue from ulcerative colitis patients obtained during surgical operation was cut with sterile scalpels into 1 cm2 pieces and placed on sterile metal grids. The tissue was stimulated with either the short or long isoforms at 100 and 1000 ng/ml in a final volume of 20 ul. As shown in FIG. 24, only short TSLP ameliorated the ongoing inflammation in the tissue. Short TSLP also restored the production of mucous by goblet cells and normalized tissue architecture (see the formation of intestinal cripts).

### BIBLIOGRAPHY

Al-Shami, A., Spolski, R., Kelly, J., Keane-Myers, A., and Leonard, W. J. (2005). A role for TSLP in the development of inflammation in an asthma model. J Exp Med 202, 829-839.
Demehri, S., Turkoz, A., Manivasagam, S., Yockey, L. J., Turkoz, M., Kopan, R. (2012). Elevated epidermal thymic stromal lymphopoietin levels establish an antitumor environment in the skin. Cancer Cell 22, 494-505.
Di Piazza, M., Nowell, C. S., Kochm U., Durham, A. D., Radtke, F. (2012). Loss of cutaneous TSLP-dependent immune responses skews the balance of inflammation from tumor protective to tumor promoting. Cancer Cell 22, 479-93.
Ferreira, D. S., Annoni, R., Silva, L. F., Buttignol, M., Santos, A. B., Medeiros, M. C., Andrade, L. N., Yick, C. Y., Sterk, P. J., Sampaio, J. L., et al. (2012). Toll-like receptors 2, 3 and 4 and thymic stromal lymphopoietin expression in fatal asthma. Clinical and experimental allergy: journal of the British Society for Allergy and Clinical Immunology 42, 1459-1471.
Fontenot, D., He, H., Hanabuchi, S., Nehete, P. N., Zhang, M., Chang, M., Nehete, B., Wang, Y. H., Ma, Z. M., Lee, H. C., et al. (2009). TSLP production by epithelial cells exposed to immunodeficiency virus triggers DC-mediated mucosal infection of CD4+ T cells. Proceedings of the National Academy of Sciences of the United States of America 106, 16776-16781.
Hanabuchi, S., Ito, T., Park, W. R., Watanabe, N., Shaw, J. L., Roman, E., Arima, K., Wang, Y. H., Voo, K. S., Cao, W., and Liu, Y. J. (2010). Thymic stromal lymphopoietin-activated plasmacytoid dendritic cells induce the generation of FOXP3+ regulatory T cells in human thymus. J. Immunol. 184, 2999-3007.
Harada, M., Hirota, T., Jodo, A. I., Hitomi, Y., Sakashita, M., Tsunoda, T., Miyagawa, T., Doi, S., Kameda, M., Fujita, K., et al. (2011). Thymic stromal lymphopoietin gene promoter polymorphisms are associated with susceptibility to bronchial asthma. American journal of respiratory cell and molecular biology 44, 787-793.
He, J. Q., Hallstrand, T. S., Knight, D., Chan-Yeung, M., Tripp, B., Zamar, D., Bosse', Y., Kozyrskyj, A. L., James, A. et al. (2009). A thymic stromal lymphopoietin gene variant is associated with asthma and airway hyperresponsiveness. J Allergy Clin Immunol. 124, 222-9.

Hunninghake, G. M., Soto-Quiros, M. E., Avila, L., Kim, H. P., Lasky-Su, J., Rafaels, N., Ruczinski, I., Beaty, T. H., Mathias, R. A., Barnes, K. C., et al. (2010). TSLP polymorphisms are associated with asthma in a sex-specific fashion. Allergy 65, 1566-1575.
Iliev, I. D., Spadoni, I., Mileti, E., Matteoli, G., Sonzogni, A., Sampietro, G. M., Foschi, D., Caprioli, F., Viale, G., and Rescigno, M. (2009). Human intestinal epithelial cells promote the differentiation of tolerogenic dendritic cells. Gut 58, 1481-1489.
Ito, T., Wang, Y. H., Duramad, O., Hori, T., Delespesse, G. J., Watanabe, N., Qin, F. X., Yao, Z., Cao, W., and Liu, Y. J. (2005). TSLP-activated dendritic cells induce an inflammatory T helper type 2 cell response through OX40 ligand. J Exp Med 202, 1213-1223.
Kashyap, M., Rochman, Y., Spolski, R., Samsel, L., and Leonard, W. J. (2011). Thymic stromal lymphopoietin is produced by dendritic cells. J Immunol 187, 1207-1211.
Kinoshita, H., Takai, T., Le, T. A., Kamijo, S., Wang, X. L., Ushio, H., Hara, M., Kawasaki, J., Vu, A. T., Ogawa, T., et al. (2009). Cytokine milieu modulates release of thymic stromal lymphopoietin from human keratinocytes stimulated with double-stranded RNA. The Journal of allergy and clinical immunology 123, 179-186.
Liu, W., Xu, L. S., Liu, Q. J., Dong, F. Z., Qiu, R. F., Wen, M. C., Han, Y. L., Tang, N. B., Kang, L. J., Wu, J. X., et al. (2012). Two single nucleotide polymorphisms in TSLP gene are associated with asthma susceptibility in Chinese Han population. Exp Lung Res 38, 375-382.
Matteoli, G., Mazzini, E., Iliev, I. D., Mileti, E., Fallarino, F., Puccetti, P., Chieppa, M., Rescigno, M. (2010). Gut CD103+ dendritic cells express indoleamine 2,3-dioxygenase which influences T regulatory/T effector cell balance and oral tolerance induction. Gut 59, 595-604.
Negishi, H., Mild, S., Sarashina, H., Taguchi-Atarashi, N., Nakajima, A., Matsuki, K., Endo, N., Yanai, H., Nishio, J., Honda, K., and Taniguchi, T. (2012). Essential contribution of IRF3 to intestinal homeostasis and microbiota-mediated Tslp gene induction. Proceedings of the National Academy of Sciences of the United States of America 109, 21016-21021.
Reardon, C., Lechmann, M., Brustle, A., Gareau, M. G., Shuman, N., Philpott, D., Ziegler, S. F., and Mak, T. W. (2011). Thymic stromal lymphopoetin-induced expression of the endogenous inhibitory enzyme SLPI mediates recovery from colonic inflammation. Immunity 35, 223-235.
Rimoldi, M., Chieppa, M., Salucci, V., Avogadri, F., Sonzogni, A., Sampietro, G. M., Nespoli, A., Viale, G., Allavena, P., and Rescigno, M. (2005). Intestinal immune homeostasis is regulated by the crosstalk between epithelial cells and dendritic cells. Nat Immunol 6, 507-514.
Siracusa, M. C., Saenz, S. A., Hill, D. A., Kim, B. S., Headley, M. B., Doering, T. A., Wherry, E. J., Jessup, H. K., Siegel, L. A., Kambayashi, T., et al. (2011). TSLP promotes interleukin-3-independent basophil haematopoiesis and type 2 inflammation. Nature 477, 229-233.
Soumelis, V., Reche, P. A., Kanzler, H., Yuan, W., Edward, G., Homey, B., Gilliet, M., Ho, S., Antonenko, S., Lauerma, A., et al. (2002). Human epithelial cells trigger dendritic cell mediated allergic inflammation by producing TSLP. Nat Immunol 3, 673-680.
Spadoni, I., Iliev, I. D., Rossi, G., and Rescigno, M. (2012). Dendritic cells produce TSLP that limits the differentiation of Th17 cells, fosters Treg development, and protects against colitis. Mucosal Immunol 5, 184-193.
Taylor, B. C., Zaph, C., Troy, A. E., Du, Y., Guild, K. J., Comeau, M. R., and Artis, D. (2009). TSLP regulates intestinal immunity and inflammation in mouse models of helminth infection and colitis. J Exp Med 206, 655-667.
Watanabe, N., Hanabuchi, S., Soumelis, V., Yuan, W., Ho, S., de Waal Malefyt, R., and Liu, Y. J. (2004). Human thymic stromal lymphopoietin promotes dendritic cell-mediated CD4+ T cell homeostatic expansion. Nature immunology 5, 426-434.
Watanabe, N., Wang, Y. H., Lee, H. K., Ito, T., Cao, W., and Liu, Y. J. (2005). Hassall's corpuscles instruct dendritic cells to induce CD4+CD25+ regulatory T cells in human thymus. Nature 436, 1181-1185.
Xie, Y., Takai, T., Chen, X., Okumura, K., Ogawa, H. (2012). Long TSLP transcript expression and release of TSLP induced by TLR ligands and cytokines in human keratinocytes. J Dermatol Sci 66, 233-7.
Yadava, K., Sichelstiel, A., Luescher, I. F., Nicod, L. P., Harris, N. L., and Marsland, B. J. (2013). TSLP promotes influenza-specific CD8+ T-cell responses by augmenting local inflammatory dendritic cell function. Mucosal Immunology 6, 83-92.
Ying, S., O'Connor, B., Ratoff, J., Meng, Q., Mallett, K., Cousins, D., Robinson, D., Zhang, G., Zhao, J., Lee, T. H., and Corrigan, C. (2005). Thymic stromal lymphopoietin expression is increased in asthmatic airways and correlates with expression of Th2-attracting chemokines and disease severity. J Immunol 174, 8183-8190.
Zaph, C., Troy, A. E., Taylor, B. C., Berman-Booty, L. D., Guild, K. J., Du, Y., Yost, E. A., Gruber, A. D., May, M. J., Greten, F. R., et al. (2007). Epithelial-cell-intrinsic IKK-beta expression regulates intestinal immune homeostasis. Nature 446, 552-556.
Zhou, B., Comeau, M. R., De Smedt, T., Liggitt, H. D., Dahl, M. E., Lewis, D. B., Gyarmati, D., Aye, T., Campbell, D. J., and Ziegler, S. F. (2005). Thymic stromal lymphopoietin as a key initiator of allergic airway inflammation in mice. Nat Immunol 6, 1047-1053.
Ziegler, S. F. (2012). Thymic stromal lymphopoietin and allergic disease. The Journal of allergy and clinical immunology 130, 845-852.

1. A compound selected from the group consisting of:
a) a polypeptide being a fragment of the C-terminus amino acid sequence of the human Thymic stromal lymphopoietin (TSLP) long isoform (SEQ ID NO: 2), or a fragment of the corresponding sequence encoded from a TSLP orthologous or homologous gene, functional mutants, recombinant or synthetic derivatives, fragments or analogues thereof;
b) a polynucleotide coding for said polypeptide;
c) a vector comprising said polynucleotide; and
d) a host cell genetically engineered expressing said polypeptide.

2. The compound according to claim 1 wherein the C-terminus amino acid sequence of the TSLP long isoform (SEQ ID NO: 2) consists of the amino acid sequence of the TSLP short isoform (SEQ ID NO: 1), or of an amino acid sequence of the corresponding region encoded from a TSLP orthologous or homologous gene, functional mutants, recombinant or synthetic derivatives, fragments or analogues thereof.

3. The compound according to claim 2, wherein the polypeptide includes a sequence consisting essentially of the aa. 4-63 of the amino acid sequence of the TSLP short isoform (SEQ ID NO: 1), or an amino acid sequence of the corresponding region encoded from a TSLP orthologous or homologous gene, functional mutants, recombinant or synthetic derivatives, fragments or analogues thereof.

4. The compound according to claim 1, wherein the polypeptide comprises a sequence consisting essentially of the aa. 4-40 or aa. 24-63 of the sequence of the TSLP short isoform (SEQ ID NO: 1) or of an amino acid sequence of the corresponding region encoded from a TSLP orthologous or homologous gene, functional mutants, recombinant or synthetic derivatives, fragments or analogues thereof.

5. The compound according to claim 1, wherein the polypeptide consists of an amino acid sequence consisting essentially of aa. 4-40 or aa. 24-63 of the sequence of the TSLP short isoform (SEQ ID NO: 1), or of the an amino acid sequence of the corresponding region encoded from a TSLP orthologous or homologous gene, functional mutants, recombinant or synthetic derivatives, fragments or analogues thereof.

6. The compound according to claim 2, wherein the polypeptide consists essentially of the aa. 4-63 of the amino acid sequence of the TSLP short isoform (SEQ ID NO: 1), or of an amino acid sequence of the corresponding region encoded from a TSLP orthologous or homologous gene, functional mutants, recombinant or synthetic derivatives, fragments or analogues thereof.

7. A compound according to claim 1, having an immunomodulatory activity.

8. The compound according to claim 7, wherein the immunomodulatory activity is an anti-inflammatory activity.

9-12. (canceled)

13. The compound according to claim 1, wherein said derivatives are selected from the group consisting of polypeptides having a percentage of identity of at least 41% with SEQ ID NO:1 or with an amino acid sequence of the corresponding region encoded from a TSLP orthologous or homologous gene.

14. The compound according to claim 1 being a synthetic derivative of the polypeptide.

15. The compound according to claim 1, wherein said fragment refers to a polypeptide having a length of at least 10 amino acids.

16. The compound according to claim 1, wherein the polynucleotide comprises a sequence which encodes the sequence SEQ ID NO:1.

17. The compound according to claim 1, wherein the vector is an expression vector selected from the group consisting of: plasmids, viral particles and phages.

18. The compound according to claim 1, wherein the host cell is selected from the group consisting of: bacterial cells, fungal cells, insect cells, animal cells, and plant cells.

19-21. (canceled)

22. A pharmaceutical composition comprising at least one pharmaceutically acceptable excipient and a compound according to claim 1, optionally further comprising at least one immunomodulatory agent.

23. (canceled)

24. The pharmaceutical composition according to claim 22 for systemic, oral, locally, rectally, or topical administration.

25. A method for the diagnosis and/or prognosis of a disorder or pathology characterized by an inflammatory response and/or for the monitoring of disorder or pathology characterized by an inflammatory response, and/or for the monitoring the efficacy of a therapeutic treatment of a disorder or pathology characterized by an inflammatory response and/or for the screening of a therapeutic treatment of a disorder or pathology characterized by an inflammatory response in a subject comprising the steps of:
measuring the amount of at least one polypeptide consisting of:
a) the C-terminus amino acid sequence of the human TSLP long isoform (SEQ ID NO: 2), or the corresponding sequence encoded from a TSLP orthologous or homologous gene, functional mutants, recombinant derivatives, fragments or analogues thereof and/or
b) the human TSLP long isoform (SEQ ID NO: 2) or the corresponding protein encoded from a TSLP orthologous or homologous gene, functional mutants, recombinant derivatives, fragments or analogues thereof
or measuring the amount of at least one polynucleotide coding for one or more of said polypeptide
in an isolated biological sample obtained from a subject and
comparing the same with a value from a control sample.

26. The method according to claim 25 further comprising the step of calculating the ratio between the amount of a) and b) according to the following formula: amount of a)/amount of b).

27. The method according to claim 25, wherein the step of measuring the amount of a) and/or b) comprises:
contacting the biological sample obtained from the subject with at least one antibody capable of binding selectively to:
a) the C-terminus amino acid sequence of the human TSLP long isoform (SEQ ID NO: 2), or the corresponding sequence encoded from a TSLP orthologous or homologous gene, functional mutants, recombinant derivatives, fragments or analogues thereof and/or
b) the human TSLP long isoform (SEQ ID NO: 2) or the corresponding protein encoded from a TSLP orthologous or homologous gene, functional mutants, recombinant derivatives, fragments or analogues thereof,
under conditions for the formation of an antibody-antigen complex and,
detecting said complex.

28. Kit for the diagnosis and/or prognosis of a disorder or pathology characterized by an inflammatory response, and/or for the monitoring of disorder or pathology characterized by an inflammatory response, and/or for the monitoring the efficacy of a therapeutic treatment of a disorder or pathology characterized by an inflammatory response and/or for the screening of a therapeutic treatment of a disorder or pathology characterized by an inflammatory response, comprising:
means to measure the amount of at least one polypeptide consisting of:
a) the C-terminus amino acid sequence of the human TSLP long isoform (SEQ ID NO: 2), or the corresponding sequence encoded from a TSLP orthologous or homologous gene, functional mutants, recombinant derivatives, fragments or analogues thereof and/or
b) the human TSLP long isoform (SEQ ID NO: 2) or the corresponding protein encoded from a TSLP orthologous or homologous gene, functional mutants, recombinant derivatives, fragments or analogues thereof,
and/or means to measure the amount of at least one polynucleotide coding for one or more of said polypeptide
and optionally,
control means.

29. A method for treating and/or preventing a disorder or pathology characterized by an inflammatory response comprising administering to a subject in need thereof an effective amount of a compound selected from the group consisting of:
a) a polypeptide consisting of the C-terminus amino acid sequence of the human Thymic stromal lymphopoietin (TSLP) long isoform (SEQ ID NO: 2), or a polypeptide consisting of the corresponding sequence encoded from a TSLP orthologous or homologous gene, functional mutants, recombinant or synthetic derivatives, fragments or analogues thereof;
b) a polynucleotide coding for said polypeptide;
c) a vector comprising said polynucleotide; and
d) a host cell genetically engineered expressing said polypeptide.

30. The method according to any one of claim 25, wherein the fragment of the C-terminus amino acid sequence of the human TSLP long isoform (SEQ ID NO: 2) is the polypeptide according to claim 1.

31. A method for treating and/or preventing a disorder or pathology characterized by an inflammatory response comprising administering to a subject in need thereof an effective amount of a TSLP long isoform (SEQ ID NO:2) antagonist wherein said antagonist is one of:
an antibody or antibody fragment capable of binding selectively to the TSLP long isoform (SEQ ID NO:2) or its fragments and of neutralizing the activity of the TSLP long isoform:
an antibody or antibody fragment capable of binding selectively to the TSLP long isoform (SEQ ID NO:2) receptor; or
an isolated TSLP long isoform receptor.

32. The method according to claim 29, wherein the disorder or pathology is Th1 and/or Th2-related.

33. The method according to claim 29, wherein the disorder or pathology is selected from the group consisting of: inflammatory bowel disease, colorectal cancer, psoriasis, atopic dermatitis, sepsis, sarcoidosis, condylomata accuminata, lichen ruber, basal cell carcinoma, actinic keratosis, lupus erythematodes, brain inflammation, allergy, allograft rejection and carcinoma.

34. An antibody or antigen-binding fragment thereof having binding specificity for human TSLP long isoform (SEQ ID NO: 1), wherein said antibody or antigen-binding fragment specifically binds human TSLP long isoform but does not bind human TSLP short isoform (SEQ ID NO:2).
